SEQUENCE LISTING (1 sequence)

SEQ ID NO: 1 - DNA, 1513 bp, Yersinia entomophaga
agtttgatcctggctcagattgaacgctggcggcaggcctaacacatgcaagtcgagcggcagcggaaagtagcttgctactttgccggcgagcggcggacgggtgagtaatgtctgggaaactgcctgatggagggggataactactggaaacggtagctaataccgcataacctcgcaagagcaaagtgggggaccttagggcctcacgccatcggatgtgcccagatgggattagctagtaggtggggtaatggctcacctaggcgacgatccctagctggtctgagaggatgaccagccacactggaactgagacacggtccagactcctacgggaggcagcagtggggaatattgcacaatgggcgcaagcctgatgcagccatgccgcgtgtgtgaagaaggccttcgggttgtaaagcactttcagcgaggaggaaggcatttcacctaatacgtgaagtgattgacgttactcgcagaagaagcaccggctaactccgtgccagcagccgcggtaatacggagggtgcaagcgttaatcggaattactgggcgtaaagcgcacgcaggcggtttgttaagtcagatgtgaaatccccgagcttaacttgggaactgcatttgaaactggcaagctagagtcttgtagaggggggtagaattccaggtgtagcggtgaaatgcgtagagatctggaggaataccggtggcgaaggcggccccctggacaaagactgacgctcaggtgcgaaagcgtggggagcaaacaggattagataccctggtagtccacgctgtaaacgatgtcgacttggaggttgtgcccttgaggcgtggcttccggagctaacgcgttaagtcgaccgcctggggagtacggccgcaaggttaaaactcaaatgaattgacgggggcccgcacaagcggtggagcatgtggtttaattcgatgcaacgcgaagaaccttacctactcttgacatccacagaacgtagcagagatgcttcggtgccttcgggaactgtgagacaggtgctgcatggctgtcgtcagctcgtgttgtgaaatgttgggttaagtcccgcaacgagcgcaacccttatcctttgttgccagcacgtcatggtgggaactcaagggagactgccggtgataaaccggaggaaggtggggatgacgtcaagtcatcatggcccttacgagtagggctacacacgtgctacaatggcagatacaaagtgaagcgaactcgcgagagcaagcggaccacataaagtctgtcgtagtccggattggagtctgcaactcgactccatgaagtcggaatcgctagtaatcgtagatcagaatgctacggtgaatacgttcccgggccttgtacacaccgcccgtcacaccatgggagtgggttgcaaaagaagtaggtagcttaaccttcgggagggcgcttaccactttgtgattcatgactggggtgaagtcgtaacaaggtaaccgtagaattctt

(19) United States
(12) Patent Application Publication
(10) Pub. No.: US 2021/0352912 A1
(43) Pub. Date: Nov. 18, 2021

(54) PESTICIDAL COMBINATIONS OF YERSINIA AND BACILLUS

(71) Applicant: NOVOZYMES BIOAG A/S, Bagsvaerd (DK)

(72) Inventors: Jarrod Leland, Blacksburg, VA (US); Calum Russell, Durham, NC (US)

(73) Assignee: NOVOZYMES BIOAG A/S, Bagsvaerd (DK)

(21) Appl. No.: 17/278,410

(22) PCT Filed: Sep. 19, 2019
(86) PCT No.: PCT/US2019/051951

§ 371 Date: Mar. 22, 2021

Related U.S. Application Data
(60) Provisional application No. 62/734,335, filed on Sep. 21, 2018.

(51) Int. Cl. A01N 63/20; A01N 63/23; A01P 7/04

(52) U.S. Cl. CPC A01N 63/20; A01P 7/04; A01N 63/23

(57) ABSTRACT

Disclosed herein are pesticicial combinations, including combinations of Yersinia entomophaga (and/or toxins therefrom) and/or Yersinia nurmii (and/or toxins therefrom) with Bacillus thuringiensis (and/or toxins therefrom). As shown herein, such combinations may provide unexpected pesticidal effects and may be useful for treating insects and other pests and for enhancing plant growth and/or yield.

### CROSS-REFERENCE TO A RELATED APPLICATION

This application claims priority to U.S. Provisional Patent Application No. 62/734,335, filed Sep. 21, 2018, the disclosure of each is incorporated herein by reference in its entirety.

### REFERENCE TO A SEQUENCE LISTING

This application contains a Sequence Listing in computer readable format, which is incorporated herein by reference.

### REFERENCE TO DEPOSITS OF BIOLOGICAL MATERIALS

The present disclosure contains references to biological materials deposited under the terms of the Budapest Treaty on the International Recognition of the Deposit of Microorganisms for the Purposes of Patent Procedure at the Agricultural Research Service Culture Collection, 1815 North University Street, Peoria, Ill. 61604, U.S.A.

The following biological material has been deposited on Mar. 15, 2018, under the terms of the Budapest Treaty with the Agricultural Research Service Patent Culture Collection (NRRL), Agricultural Research Service, U.S. Department of Agriculture, 1815 North University Street, Peoria, Ill. 61604, USA, and identified as follows: Yersinia entomophaga strain O43NEW (NRRL B-67598), Yersinia entomophaga strain O24G3R (NRRL B-67599), Yersinia entomophaga strain O24KEK (NRRL B-67600) and Yersinia entomophaga strain O333A4 (NRRL B-67601).

### BACKGROUND

Yersinia entomophaga is a Gram-negative bacterium that exhibits pesticidal activity against a wide range of targets, including, but not limited to, coleopteran, dipteran, hymenopteran, lepidopteran orthopteran and thysanopteran insects. See, e.g., Hurst et al., INTL. J. SYS. EVOL. MICROBIOL. 61:844 (2011); WO 2007/142543; WO 2008/041863. Previous studies have shown that Yersinia entomophaga may be used synergistically with myriad chemical pesticides. See, e.g., WO 2018/175677).

### SUMMARY

The present disclosure provides novel and inventive uses and methods for Yersinia entomophaga (and Yersinia nurmii), as well as compositions useful for practicing such methods.

A first aspect of the present disclosure is use of Yersinia entomophaga, a cell-free Yersinia entomophaga culture filtrate, or an isolated Yersinia entomophaga toxin for improving the insecticidal efficacy of a Bacillus thuringiensis, a cell-free Bacillus thuringiensis culture filtrate, or an isolated Bacillus thuringiensis toxin against a pest.

A second aspect of the present disclosure is use of Yersinia nurmii, a cell-free Yersinia nurmii culture filtrate, or an isolated Yersinia nurmii toxin for improving the insecticidal efficacy of a Bacillus thuringiensis, a cell-free Bacillus thuringiensis culture filtrate, or an isolated Bacillus thuringiensis toxin against a pest.

A third aspect of the present disclosure is use of Bacillus thuringiensis, a cell-free Bacillus thuringiensis culture filtrate, or an isolated Bacillus thuringiensis toxin for improving the insecticidal efficacy of a Yersinia entomophaga, a cell-free Yersinia entomophaga culture filtrate, or an isolated Yersinia entomophaga toxin against a pest.

A fourth aspect of the present disclosure is use of Bacillus thuringiensis, a cell-free Bacillus thuringiensis culture filtrate, or an isolated Bacillus thuringiensis toxin for improving the insecticidal efficacy of a Yersinia nurmii, a cell-free Yersinia nurmii culture filtrate, or an isolated Yersinia nurmii toxin against a pest.

A fifth aspect of the present disclosure is a method comprising applying one or more Yersinia entomophaga, one or more cell-free Yersinia entomophaga culture filtrates and/or one or more isolated Yersinia entomophaga toxins, as well as one or more Bacillus thuringiensis, one or more cell-free Bacillus thuringiensis culture filtrates and/or one or more isolated Bacillus thuringiensis toxins to a plant or plant part.

A sixth aspect of the present disclosure is a method comprising applying one or more Yersinia nurmii, one or more cell-free Yersinia nurmii culture filtrates and/or one or more isolated Yersinia nurmii toxins, as well as one or more Bacillus thuringiensis, one or more cell-free Bacillus thuringiensis culture filtrates and/or one or more isolated Bacillus thuringiensis toxins to a plant or plant part.

A seventh aspect of the present disclosure is a method comprising applying one or more Yersinia entomophaga, one or more cell-free Yersinia entomophaga culture filtrates and/or one or more isolated Yersinia entomophaga toxins to a plant or plant part that expresses one or more Bacillus thuringiensis toxins.

An eighth aspect of the present disclosure is a method comprising applying one or more Yersinia nurmii, one or more cell-free Yersinia nurmii culture filtrates and/or one or more isolated Yersinia nurmii toxins to a plant or plant part that expresses one or more Bacillus thuringiensis toxins.

A ninth aspect of the present disclosure is a method comprising applying one or more Bacillus thuringiensis, one or more cell-free Bacillus thuringiensis culture filtrates and/or one or more isolated Bacillus thuringiensis toxins to a plant or plant part that expresses one or more Yersinia entomophaga toxins or components thereof (e.g., Chi1, Chi2, YenA1, YenA2, YenB, YenC1 and/or YenC2).

A tenth aspect of the present disclosure is a method comprising applying one or more Bacillus thuringiensis, one or more cell-free Bacillus thuringiensis culture filtrates and/or one or more isolated Bacillus thuringiensis toxins to a plant or plant part that expresses one or more Yersinia nurmii toxins or components thereof.

An eleventh aspect of the present disclosure is synthetic microbial consortium comprising, consisting essentially of or consisting of one or more strains of Yersinia entomophaga and/or one or more strains of Yersinia nurmii and/or one or more strains of Bacillus thuringiensis.

A twelfth aspect of the present disclosure is composition comprising, consisting essentially of, or consisting of one or more strains of Yersinia entomophaga (and/or one or more toxins or other components derived therefrom) and/or one or more strains of Yersinia nurmii (and/or one or more toxins or other components derived therefrom) and/or one or more strains of Bacillus thuringiensis (and/or one or more toxins or other components derived therefrom).

A thirteenth aspect of the present disclosure is a transgenic plant comprising genetic material derived from Yersinia entomophaga and/or Yersinia nurmii and/or Bacillus thuringiensis.

A fourteenth aspect of the present disclosure is a transgenic plant that expresses one or more toxins of/from Yersinia entomophaga and/or Yersinia nurmii.

### DETAILED DESCRIPTION

This description is not intended to be a detailed catalog of all the different ways in which the invention may be implemented or of all the features that may be added to the instant invention. For example, features illustrated with respect to one embodiment may be incorporated into other embodiments and features illustrated with respect to a particular embodiment may be deleted from that embodiment. In addition, numerous variations and additions to the various embodiments suggested herein, which do not depart from the instant invention, will be apparent to those skilled in the art in light of the instant disclosure. Hence, the following description is intended to illustrate some particular embodiments of the invention and not to exhaustively specify all permutations, combinations and variations thereof.

The terminology used herein is for the purpose of describing particular embodiments only and is not intended to be limiting of the invention.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the specification and relevant art and should not be interpreted in an idealized or overly formal sense unless expressly so defined herein. For the sake of brevity and/or clarity, well-known functions or constructions may not be described in detail.

As used herein, the singular forms “a,” “an,” and “the” are intended to include the plural forms as well, unless the context clearly indicates otherwise.

As used herein, the terms “acaricide” and “acaricidal” refer to an agent or combination of agents the application of which is toxic to an acarid (i.e., kills an acarid, inhibits the growth of an acarid and/or inhibits the reproduction of an acarid).

As used herein, there term “additive”, when used in reference to a combination of two or more independent effects (e.g., a first effect produced by application of a Yersinia entomophaga and a second effect produced by application of a Bacillus thuringiensis), means the cumulative effect of the two or more independant effects is (about) the same as the theoretical sum of the independent effects in combination.

As used herein, the term “agriculturally acceptable carrier” refers to a substance or composition that can be used to deliver an agriculturally beneficial agent to a plant, plant part or plant growth medium (e.g., soil) without causing/having an unduly adverse effect on plant growth and/or yield. As used herein, the term “foliar-compatible carrier” refers to a material that can be foliarly applied to a plant or plant part without causing/having an unduly adverse effect on the plant, plant part, plant growth, plant health, or the like. As used herein, the term “seed-compatible carrier” refers to a material that can be applied to a seed without causing/having an unduly adverse effect on the seed, the plant that grows from the seed, seed germination, or the like. As used herein, the term “soil-compatible carrier” refers to a material that can be added to a soil without causing/having an unduly adverse effect on plant growth, soil structure, soil drainage, or the like.

As used herein, the term “agriculturally beneficial agent” refers to any agent (e.g., chemical or biological agent) or combination of agents the application of which causes or provides a beneficial and/or useful effect in agriculture including, but not limited to, agriculturally beneficial microorganisms, biostimulants, nutrients, pesticides (e.g., acaricides, fungicides, herbicides, insecticides, and nematicides) and plant signal molecules.

As used herein, the term “agriculturally beneficial microorganism” refers to a microorganism having at least one agriculturally beneficial property (e.g., the ability to fix nitrogen, the ability to solubilize phosphate and/or the ability to produce an agriculturally beneficial agent, such as a plant signal molecule).

As used herein, the term “and/or” is intended to include any and all combinations of one or more of the associated listed items, as well as the lack of combinations when interpreted in the alternative (“or”). Thus, the phrase “A, B and/or C” is to be interpreted as “A, A and B, A and B and C, A and C, B, B and C, or C.”

As used herein, there term “antagonistic”, when used in reference to a combination of two or more independent effects (e.g., a first effect produced by application of a Yersinia entomophaga and a second effect produced by application of a Bacillus thuringiensis), means the cumulative effect of the two or more independant effects is decreased as compared to the theoretical sum of the independent effects in combination.

As used herein, the terms “associated with,” in association with” and “associated therewith,” when used in reference to a relationship between a microbial strain or inoculant composition of the present disclosure and a plant or plant part, refer to at least a juxtaposition or close proximity of the microbial strain or inoculant composition and the plant or plant part. Such a juxtaposition or close proximity may be achieved by contacting or applying the microbial strain or inoculant composition directly to the plant or plant part and/or by applying the microbial strain or inoculant composition to the plant growth medium (e.g., soil) in which the plant or plant part will be grown (or is currently being grown). According to some embodiments, the microbial strain or inoculant composition is applied as a coating to the outer surface of the plant or plant part. According to some embodiments, the microbial strain or inoculant composition is applied to soil at, near or surrounding the site in which the plant or plant part will be grown (or is currently being grown).

As used herein, the term “aqueous” refers to a composition that contains more than a trace amount of water (i.e., more than 0.5% water by weight, based upon the total weight of the composition).

As used herein, the term “biologically pure culture” refers to a microbial culture that is free or essentially free of biological contamination and that has genetic uniformity such that different subculutres taken therefrom will exhibit identicial or substantially identical genotyopes and phenotypes. In some embodiments, the biologically pure culture is 100% pure (i.e., all subcultures taken therefrom exhibit identical genotypes and phenotypes). In some embodiments, the biologically pure culture is at least 90, 91, 92, 93, 94, 95, 96, 97, 98, 99, 99.5, 99.6, 99.7, 99.8, or 99.9% pure (i.e., at least 90, 91, 92, 93, 94, 95, 96, 97, 98, 99, 99.5, 99.6, 99.7, 99.8, or 99.9% of the subcultures taken therefrom exhibit identical genotypes and phenotypes).

As used herein, the term “biostimulant” refers to an agent or combination of agents the application of which enhances one or more metabolic and/or physiological processes of a plant or plant part (e.g., carbohydrate biosynthesis, ion uptake, nucleic acid uptake, nutrient delivery, photosynthesis and/or respiration).

As used herein, the term “BRADY” is to be interpreted as a shorthand substitute for the phrase “Bradyrhizobium spp. 8A57, Bradyrhizobium elkanii SEMIA 501, Bradyrhizobium elkanii SEMIA 587, Bradyrhizobium elkanii SEMIA 5019, Bradyrhizobium japonicum 61A227, Bradyrhizobium japonicum 61A228, Bradyrhizobium japonicum 61A273, Bradyrhizobium japonicum E-109, Bradyrhizobium japonicum NRRL B-50586 (also deposited as NRRL B-59565), Bradyrhizobium japonicum NRRL B-50587 (also deposited as NRRL B-59566), Bradyrhizobium japonicum NRRL B-50588 (also deposited as NRRL B-59567), Bradyrhizobium japonicum NRRL B-50589 (also deposited as NRRL B-59568), Bradyrhizobium japonicum NRRL B-50590 (also deposited as NRRL B-59569), Bradyrhizobium japonicum NRRL B-50591 (also deposited as NRRL B-59570), Bradyrhizobium japonicum NRRL B-50592 (also deposited as NRRL B-59571), Bradyrhizobium japonicum NRRL B-50593 (also deposited as NRRL B-59572), Bradyrhizobium japonicum NRRL B-50594 (also deposited as NRRL B-50493), Bradyrhizobium japonicum NRRL B-50608, Bradyrhizobium japonicum NRRL B-50609, Bradyrhizobium japonicum NRRL B-50610, Bradyrhizobium japonicum NRRL B-50611, Bradyrhizobium japonicum NRRL B-50612, Bradyrhizobium japonicum NRRL B-50726, Bradyrhizobium japonicum NRRL B-50727, Bradyrhizobium japonicum NRRL B-50728, Bradyrhizobium japonicum NRRL B-50729, Bradyrhizobium japonicum NRRL B-50730, Bradyrhizobium japonicum SEMIA 566, Bradyrhizobium japonicum SEMIA 5079, Bradyrhizobium japonicum SEMIA 5080, Bradyrhizobium japonicum USDA 6, Bradyrhizobium japonicum USDA 110, Bradyrhizobium japonicum USDA 122, Bradyrhizobium japonicum USDA 123, Bradyrhizobium japonicum USDA 127, Bradyrhizobium japonicum USDA 129 and/or Bradyrhizobium japonicum USDA 532C.”

As used herein, the terms “colony forming unit” and “cfu” refer to a microbial cell/spore capable of propagating on or in a suitable growth medium or substrate (e.g., a soil) when conditions (e.g., temperature, moisture, nutrient availability, pH, etc.) are favorable for germination and/or microbial growth.

As used herein, the term “consists essentially of,”, when used in reference to inoculant compositions and methods of the present disclosure, means that the compositions/methods may contain additional components/steps so long as the additional components/steps do not materially alter the composition/method. The term “materially alter,” as applied to a composition/method of the present disclosure, refers to an increase or decrease in the effectiveness of the composition/method of at least 20%. For example, a component added to an inoculant composition of the present disclosure may be deemed to “materially alter” the composition if it increases or decreases the composition's ability to enhance plant yield by at least 20%.

As used herein, the term “diazotroph” refers to an organism capable of converting atmospheric nitrogen (N₂) into a form that may be utilized by a plant or plant part (e.g., ammonia (NH₃), ammonium (NH₄+), etc.).

As used herein, the term “dispersant” refers to an agent or combination of agents the application of which reduces the cohesiveness of like particles, the surface tension of a liquid, the interfacial tension between two liquids and/or the interfacial tension between or a liquid and a solid.

As used herein, the terms “effective amount,” “effective concentration” and “effective amount/concentration” refer to an amount or concentration that is sufficient to cause a desired effect (e.g. toxicity to a pest and/or enhanced crop yield). The absolute value of the amount/concentration that is sufficient to cause the desired effect may be affected by factors such as the type and magnitude of effect desired, the type, size and volume of material to which the inoculant compositon will be applied, the type(s) of microorganisms in the composition, the number of microorganisms in the composition, the stability of the microorganism(s) in the inoculant composition and the storage conditions (e.g., temperature, relative humidity, duration). Those skilled in the art will understand how to select an effective amount/concentration using routine dose-response experiments.

As used herein, the term “enhanced dispersion” refers to an improvement in one or more characteristics of microbial dispersion as compared to one or more controls (e.g., a control composition that is identical to an inoculant composition of the present disclosure except that it lacks one or more of the components found in the inoculant composition of the present disclosure). Exemplary microbial dispersion characteristics include, but are not limited to, the percentage of microbes that exist as single cells/spores when the inoculant composition is diluted in water. An inoculant composition that improves one or more microbial dispersion characteristics of the microorganism(s) contained therein as compared to a control composition (e.g., a control composition that is identical to the inoculant composition except that it lacks one or more of the components found in the inoculant composition) provides enhanced dispersion and can be referred to as a “readily dispersable inoculant composition.”

As used herein, the terms “enhanced growth” and “enhanced plant growth” refer to an improvement in one or more characteristics of plant growth and/or development as compared to one or more control plants (e.g., a plant germinated from an untreated seed or an untreated plant). Exemplary plant growth/development characteristics include, but are not limited to, biomass, carbohydrate biosynthesis, chlorophyll content, cold tolerance, drought tolerance, height, leaf canopy, leaf length, leaf mass, leaf number, leaf surface area, leaf volume, lodging resistance, nutrient uptake and/or accumulation (e.g., ammonium, boron, calcium, copper, iron, magnesium, manganese, nitrate, nitrogen, phosphate, phosphorous, potassium, sodium, sulfur and/or zinc uptake/accumulation), rate(s) of photosynthesis, root area, root diameter, root length, root mass, root nodulation (e.g., nodule mass, nodule number, nodule volume), root number, root surface area, root volume, salt tolerance, seed germination, seedling emergence, shoot diameter, shoot length, shoot mass, shoot number, shoot surface area, shoot volume, spread, stand, stomatal conductance and survival rate. Unless otherwise indicated, references to enhanced plant growth are to be interpreted as meaning that microbial strains, inoculant compositions and methods of the present disclosure enhance plant growth by enhancing nutrient availability, improving soil characteristics, etc. and are not to be interpreted as suggesting that microbial strains, inoculant compositions and methods of the present disclosure act as plant growth regulators.

As used herein, the terms “enhanced stability” and “enhanced microbial stability” refer to an improvement in one or more characteristics of microbial stability as compared to one or more controls (e.g., a control composition that is identical to an inoculant composition of the present disclosure except that it lacks one or more of the components found in the inoculant composition of the present disclosure). Exemplary microbial stability characteristics include, but are not limited to, the ability to germinate and/or propagate after being coated on a seed and/or stored for a defined period of time and the ability to cause a desired effect (e.g., enhanced plant yield and/or increased pesticidal activity) after being coated on a seed and/or stored for a defined period of time. A microorganism that exhibits improvement in one or more microbial stability characteristics as compared to a control microorganism when each is subjected to the same conditions (e.g., seed coating and storage conditions) displays enhanced stability and can be referred to as a “stable microorganism.” An inoculant composition that improves one or more microbial stability characteristics of the microorganism(s) contained therein as compared to a control composition (e.g., a control composition that is identical to the inoculant composition except that it lacks one or more of the components found in the inoculant composition) provides enhanced stability and can be referred to as a “stable inoculant composition.”

As used herein, the terms “enhanced survival” and “enhanced microbial survival” refer to an improvement in the survival rate of one or more microorganisms in an inoculant composition as compared to one or more microorganisms in a control composition (e.g., a control composition that is identical to an inoculant composition of the present disclosure except that it lacks one or more of the components found in the inoculant composition of the present disclosure). An inoculant composition that improves the survival rate of one or more of the microorganisms contained therein as compared to a control composition (e.g., a control composition that is identical to the inoculant composition except that it lacks one or more of the components found in the inoculant composition) provides enhanced survival and can be referred to as a stable inoculant composition.

As used herein, the terms “enhanced yield” and “enhanced plant yield” refer to an improvement in one or more characteristics of plant yield as compared to one or more control plants (e.g., a control plant germinated from an untreated seed). Exemplary plant yield characteristics include, but are not limited to, biomass; bushels per acre; grain weight per plot (GWTPP); nutritional content; percentage of plants in a given area (e.g., plot) that fail to produce grain; yield at standard moisture percentage (YSMP), such as grain yield at standard moisture percentage (GYSMP); yield per plot (YPP), such as grain weight per plot (GWTPP); and yield reduction (YRED). Unless otherwise indicated, references to enhanced plant yield are to be interpreted as meaning that microbial strains, inoculant compositions and methods of the present disclosure enhance plant yield by enhancing nutrient availability, improving soil characteristics, etc. and are not to be interpreted as suggesting that microbial strains, inoculant compositions and methods of the present disclosure act as plant growth regulators.

As used herein, the term “foliage” refers to those portions of a plant that normally grow above the ground, including, but not limited to, leaves, stalks, stems, flowers, fruiting bodies and fruits.

As used herein, the terms “foliar application” and “foliarly applied” refer to the application of one or more active ingredients to the foliage of a plant (e.g., to the leaves of the plant). Application may be effected by any suitable means, including, but not limited to, spraying the plant with a composition comprising the active ingredient(s). In some embodiments, the active ingredient(s) is/are applied to the leaves, stems and/or stalk of the plant and not to the flowers, fruiting bodies or fruits of the plant.

As used herein, the terms “fungicide” and “fungicidal” refer to an agent or combination of agents the application of which is toxic to a fungus (i.e., kills a fungus, inhibits the growth of a fungus and/or inhibits the reproduction of a fungus).

As used herein, the term “gastropodicide” refers to an agent or combination of agents the application of which is toxic to a gastropod (e.g., snails, slugs) (kills a gastropod, inhibits the growth of a gastropod and/or inhibits the reproduction of a gastropod). The term “molluscicide” is generally interchangeable with gastropodicide.

As used herein, the term “fulvic acid” encompasses pure fulvic acids and fulvic acid salts (fulvates). Non-limiting examples of fulvic acids include ammonium fulvate, boron fulvate, potassium fulvate, sodium fulvate, etc. In some embodiments, the fulvic acid comprises, consists essentially of or consists MDL Number MFCD09838488 (CAS Number 479-66-3).

As used herein, the terms “herbicide” and “herbicidal” refer to an agent or combination of agents the application of which is toxic to a weed (i.e., kills a weed, inhibits the growth of a weed and/or inhibits the reproduction of a weed).

As used herein, the term “humic acid” encompasses pure humic acids and humic acid salts (humates). Non-limiting examples of humic acids include ammonium humate, boron humate, potassium humate, sodium humate, etc. In some embodiments, the humic acid comprises, consists essentially of or consists of one or more of MDL Number MFCD00147177 (CAS Number 1415-93-6), MDL Number MFCD00135560 (CAS Number 68131-04-4), MDL Number MFCS22495372 (CAS Number 68514-28-3), CAS Number 93924-35-7 and CAS Number 308067-45-0.

As used herein, the terms “inoculant composition” and “inoculum” refer to a composition comprising microbial cells and/or spores, said cells/spores being capable of propagating/germinating on or in a suitable growth medium or substrate (e.g., a soil) when conditions (e.g., temperature, moisture, nutrient availability, pH, etc.) are favorable for germination and/or microbial growth.

As used herein, the terms “insecticide” and “insecticidal” refer to an agent or combination of agents the application of which is toxic to an insect (i.e., kills an insect, inhibits the growth of an insect and/or inhibits the reproduction of an insect).

As used herein, the term “isolated microbial strain” refers to a microbe that has been removed from the environment in which it is normally found.

As used herein, the term “isomer” includes all stereoisomers of the compounds and/or molecules to which it refers, including enantiomers and diastereomers, as well as all conformers, roatmers and tautomers, unless otherwise indicated. Compounds and/or molecules disclosed herein include all enantiomers in either substantially pure levorotatory or dextrorotatory form, or in a racemic mixture, or in any ratio of enantiomers. Where embodiments disclose a (D)-enantiomer, that embodiment also includes the (L)-enantiomer; where embodiments disclose a (L)-enantiomer, that embodiment also includes the (D)-enantiomer. Where embodiments disclose a (+)-enantiomer, that embodiment also includes the (−)-enantiomer; where embodiments disclose a (−)-enantiomer, that embodiment also includes the (+)-enantiomer. Where embodiments disclose a (S)-enantiomer, that embodiment also includes the (R)-enantiomer; where embodiments disclose a (R)-enantiomer, that embodiment also includes the (S)-enantiomer. Embodiments are intended to include any diastereomers of the compounds and/or molecules referred to herein in diastereomerically pure form and in the form of mixtures in all ratios. Unless stereochemistry is explicitly indicated in a chemical structure or chemical name, the chemical structure or chemical name is intended to embrace all possible stereoisomers, conformers, rotamers and tautomers of compounds and/or molecules depicted.

As used herein, the terms “miticide” and “miticidal” refer to an agent or combination of agents the application of which is toxic to an mite (i.e., kills a mite, inhibits the growth of a mite and/or inhibits the reproduction of a mite).

As used herein, the term “modified microbial strain” refers to a microbial strain that is modified from a strain isolated from nature. Modified microbial strains may be produced by any suitable method(s), including, but not limited to, chemical or other form of induced mutation to a polynucleotide within any genome within the strain; the insertion or deletion of one or more nucleotides within any genome within the strain, or combinations thereof; an inversion of at least one segment of DNA within any genome within the strain; a rearrangement of any genome within the strain; generalized or specific transduction of homozygous or heterozygous polynucleotide segments into any genome within the strain; introduction of one or more phage into any genome of the strain; transformation of any strain resulting in the introduction into the strain of stably replicating autonomous extrachromosomal DNA; any change to any genome or to the total DNA composition within the strain isolated from nature as a result of conjugation with any different microbial strain; and any combination of the foregoing. The term modified microbial strains includes a strain with (a) one of more heterologous nucleotide sequences, (b) one or more non-naturally occurring copies of a nucleotide sequence isolated from nature (i.e., additional copies of a gene that naturally occurs in the microbial strain from which the modified microbial strain was derived), (c) a lack of one or more nucleotide sequences that would otherwise be present in the natural reference strain by for example deleting nucleotide sequence, and (d) added extrachromosomal DNA. In some embodiments, modified microbial strains comprise a combination of two or more nucleotide sequences (e.g., two or more naturally occurring genes that do not naturally occur in the same microbial strain) or comprise a nucleotide sequence isolated from nature at a locus that is different from the natural locus.

As used herein, the terms “nematicide” and “nematicidal” refer to an agent or combination of agents the application of which is toxic to a nematode (i.e., kills a nematode, inhibits the growth of a nematode and/or inhibits the reproduction of a nematode).

As used herein, the term “nitrogen fixing organism” refers to an organism capable of converting atmospheric nitrogen (N₂) into a form that may be utilized by a plant or plant part (e.g., ammonia (NH₃), ammonium (NH₄⁺), etc.).

As used herein, the term “non-aqueous” refers to a composition that comprises no more than a trace amount of water (i.e., no more than 0.5% water by weight, based upon the total weight of the composition).

As used herein, the term “nutrient” refers to a compound or element useful for nourishing a plant (e.g., vitamins, macrominerals, micronutrients, trace minerals, organic acids, etc. that are necessary for plant growth and/or development).

As used herein, the term “PENI” is to be interpreted as a shorthand substitute for the phrase “Penicillium bilaiae ATCC 18309, Penicillium bilaiae ATCC 20851, Penicillium bilaiae ATCC 22348, Penicillium bilaiae NRRL 50162, Penicillium bilaiae NRRL 50169, Penicillium bilaiae NRRL 50776, Penicillium bilaiae NRRL 50777, Penicillium bilaiae NRRL 50778, Penicillium bilaiae NRRL 50777, Penicillium bilaiae NRRL 50778, Penicillium bilaiae NRRL 50779, Penicillium bilaiae NRRL 50780, Penicillium bilaiae NRRL 50781, Penicillium bilaiae NRRL 50782, Penicillium bilaiae NRRL 50783, Penicillium bilaiae NRRL 50784, Penicillium bilaiae NRRL 50785, Penicillium bilaiae NRRL 50786, Penicillium bilaiae NRRL 50787, Penicillium bilaiae NRRL 50788, Penicillium bilaiae RS7B-SD1, Penicillium brevicompactum AgRF18, Penicillium canescens ATCC 10419, Penicillium expansum ATCC 24692, Penicillium expansum YT02, Penicillium fellatanum ATCC 48694, Penicillium gaesfrivorus NRRL 50170, Penicillium glabrum DAOM 239074, Penicillium glabrum CBS 229.28, Penicillium janthinellum ATCC 10455, Penicillium lanosocoeruleum ATCC 48919, Penicillium radicum ATCC 201836, Penicillium radicum FRR 4717, Penicillium radicum FRR 4719, Penicillium radicum N93/47267 and/or Penicillium raistrickii ATCC 10490.”

As used herein, the term “Penicillium bilaiae” is intended to include all iterations of the species name, such as “Penicillium bilaji” and “Penicillium bilaii.”

As used herein, the terms “percent identity,” “% identity” and “percent identical” refer to the relatedness of two or more nucleotide or amino acid sequences, which may be calculated by (i) comparing two optimally aligned sequences over a window of comparison, (ii) determining the number of positions at which the identical nucleic acid base (for nucleotide sequences) or amino acid residue (for proteins) occurs in both sequences to yield the number of matched positions, (iii) dividing the number of matched positions by the total number of positions in the window of comparison, and then (iv) multiplying this quotient by 100% to yield the percent identity. If the “percent identity” is being calculated in relation to a reference sequence without a particular comparison window being specified, then the percent identity is determined by dividing the number of matched positions over the region of alignment by the total length of the reference sequence. Accordingly, for purposes of the present invention, when two sequences (query and subject) are optimally aligned (with allowance for gaps in their alignment), the “percent identity” for the query sequence is equal to the number of identical positions between the two sequences divided by the total number of positions in the query sequence over its length (or a comparison window), which is then multiplied by 100%.

As used herein, the term “pest” includes any organism or virus that negatively affects a plant, including, but not limited to, organisms and viruses that spread disease, damage host plants and/or compete for soil nutrients. The term “pest” encompasses organisms and viruses that are known to associate with plants and to cause a detrimental effect on the plant's health and/or vigor. Plant pests include, but are not limited to, arachnids (e.g., mites, ticks, spiders, etc.), bacteria, fungi, gastropods (e.g., slugs, snails, etc.), invasive plants (e.g., weeds), insects (e.g., white flies, thrips, weevils, etc.), nematodes (e.g., root-knot nematode, soybean cyst nematode, etc.), rodents and viruses (e.g., tobacco mosaic virus (TMV), tomato spotted wilt virus (TSWV), cauliflower mosaic virus (CaMV), etc.).

As used herein, the terms “pesticide” and “pesticidal” refer to agents or combinations of agents the application of which is toxic to a pest (i.e., kills a pest, inhibits the growth of a pest and/or inhibits the reproduction of a pest). Non-limiting examples of pesticides include acaricides, fungicides, herbicides, insecticides, and nematicides, etc.

As used herein, the term “phosphate-solubilizing microorganism” refers to a microorganism capable of converting insoluble phosphate into a soluble form of phosphate.

As used herein, the term “plant” includes all plant populations, including, but not limited to, agricultural, horticultural and silvicultural plants. The term “plant” encompasses plants obtained by conventional plant breeding and optimization methods (e.g., marker-assisted selection) and plants obtained by genetic engineering, including cultivars protectable and not protectable by plant breeders' rights.

As used herein, the term “plant cell” refers to a cell of an intact plant, a cell taken from a plant, or a cell derived from a cell taken from a plant. Thus, the term “plant cell” includes cells within seeds, suspension cultures, embryos, meristematic regions, callus tissue, leaves, shoots, gametophytes, sporophytes, pollen and microspores.

As used herein, the term “plant growth regulator” refers to an agent or combination of agents the application of which accelerates or retards the growth/maturation rate of a plant through direct physiological action on the plant or which otherwise alters the behavior of a plant through direct physiological action on the plant. “Plant growth regulator” shall not be interpreted to include any agent or combination of agents excluded from the definition of “plant regulator” that is set forth section 2(v) of the Federal Insecticide, Fungicide, and Rodenticide Act (7 U.S.C. § 136(v)). Thus, “plant growth regulator” does not encompass microorganisms applied to a plant, plant part or plant growth medium for the purpose of enhancing the availability and/or uptake of nutrients, nutrients necessary to normal plant growth, soil amendments applied for the purpose of improving soil characteristics favorable for plant growth or vitamin hormone products as defined by 40 C.F.R. § 152.6(f).

As used herein, the term “plant part” refers to any part of a plant, including cells and tissues derived from plants. Thus, the term “plant part” may refer to any of plant components or organs (e.g., leaves, stems, roots, etc.), plant tissues, plant cells and seeds. Examples of plant parts, include, but are not limited to, anthers, embryos, flowers, fruits, fruiting bodies, leaves, ovules, pollen, rhizomes, roots, seeds, shoots, stems and tubers, as well as scions, rootstocks, protoplasts, calli and the like.

As used herein, the term “plant propagation material” refers to a plant part from which a whole plant can be generated. Examples of plant propagation materials include, but are not limited to, cuttings (e.g., leaves, stems), rhizomes, seeds, tubers and cells/tissues that can be cultured into a whole plant.

As used herein, the term “progeny” refers to the descendent(s) of B. velezensis NRRL B-67354 and encompasses both immediate offspring of B. velezensis NRRL B-67354 and any decendants thereof.

As used herein, the terms “spore” and “microbial spore” refer to a microorganism in its dormant, protected state.

As used herein, the term “stabilizing compound” refers to an agent or combination of agents the application of which enhances the survival and/or stability of a microorganism in an inoculant composition.

As used herein with respect to inoculant compositions, the term “stable” refers to an inoculant composition in which microorganisms exhibit enhanced stability and/or enhanced survival. In general, an inoculant composition may be labeled “stable” if it improves the survival rate and/or at least one microbial stability characteristic of at least one microorganism contained therein.

As used herein with respect to microbial strains, the term “survival rate” refers to the percentage of microbial cell/spore that are viable (i.e., capable of propagating on or in a suitable growth medium or substrate (e.g., a soil) when conditions (e.g., temperature, moisture, nutrient availability, pH, etc.) are favorable for germination and/or microbial growth) at a given period of time.

As used herein, the term “Yersinia strains of the present disclosure” encompasses Yersinia entomophaga MH96, which has previously been described (see, e.g., Hurst, M. R. H. et al., TOXINS 8:143 (2016); GenBank Accession No. DQ400782), and which has previously been deposited as in the Leibniz Institute DSMZ-German Collection of Microorganisms and Cell Cultures as DSM 22339, in the American Type Culture Collection as ATCC BAA-1678, and in the U.S. Department of Agriculture's Agricultural Research Service Culture Collection as NRRL B-67598, progeny of Y. entomophaga MH96, modified microbial strains derived from Y. entomophaga MH96, modified microbial strains derived from progeny of Y. entomophaga MH96, Yersinia entomophaga O24G3R, which has a whole genome sequence that is 99.74% identical to that of MH96 and which has previously been deposited as NRRL B-67599, progeny of Y. entomophaga O24G3R, modified microbial strains derived from Y. entomophaga O24G3R, modified microbial strains derived from progeny of Y. entomophaga O24G3R, Yersinia entomophaga O24KEK, which has a whole genome sequence that is 99.69% identical to that of MH96 and which has previously been deposited as NRRL B-67600, progeny of Y. entomophaga O24KEK, modified microbial strains derived from Y. entomophaga O24KEK, modified microbial strains derived from progeny of Y. entomophaga O24KEK, Yersinia entomophaga O333A4, which has a whole genome sequence that is 99.7% identical to that of MH96 and which has previously been deposited as NRRL B-67601, progeny of Y. entomophaga O333A4, modified microbial strains derived from Y. entomophaga O333A4, modified microbial strains derived from progeny of Y. entomophaga O333A4, Yersinia entomophaga O23ZMJ, which has a whole genome sequence that is 99.59% identical to that of MH96, progeny of Y. entomophaga O23ZMJ, modified microbial strains derived from Y. entomophaga O23ZMJ, modified microbial strains derived from progeny of Y. entomophaga O23ZMJ, Yersinia entomophaga O348UX, progeny of Y. entomophaga O348UX, modified microbial strains derived from Y. entomophaga O348UX, modified microbial strains derived from progeny of Y. entomophaga O348UX, Yersinia entomophaga O33ZDX, progeny of Y. entomophaga O33ZDX, modified microbial strains derived from Y. entomophaga O33ZDX, and modified microbial strains derived from progeny of Y. entomophaga O33ZDX. Progeny may be produced using any suitable method(s), including, but not limited to, protoplast fusion, traditional breeding programs and combinations thereof. Modified microbial strains may be produced using suitable method(s), including, but not limited to, chemically-induced mutation of a polynucleotide within any genome within one of the aformentioend strains; the insertion or deletion of one or more nucleotides within any genome within one of the aformentioend strains, or combinations thereof; an inversion of at least one segment of DNA within any genome within one of the aformentioend strains; a rearrangement of any genome within one of the aformentioend strains; generalized or specific transduction of homozygous or heterozygous polynucleotide segments into any genome within one of the aformentioend strains; introduction of one or more phage into any genome of one of the aformentioend strains; transformation of one of the aformentioend strains resulting in the introduction into one of the aformentioend strains of stably replicating autonomous extrachromosomal DNA; any change to any genome or to the total DNA composition within one of the aformentioend strains as a result of conjugation with any different microbial strain; and any combination of the foregoing.

The present disclosure extends to close relatives of Yersinia entomophaga strains of the present disclosure, including, but not limited to, closely related progeny of Y. entomophaga MH96, (e.g., progeny having a 16S sequence that is about/at least 95, 95.5, 95.55, 95.6, 95.65, 95.7, 95.75, 95.8, 95.85, 95.9, 95.95, 96, 96.05, 96.1, 96.15, 96.2, 96.25, 96.3, 96.35, 96.4, 96.45, 96.5, 96.55, 96.6, 96.65, 96.7, 96.75, 96.8, 96.85, 96.9, 96.95, 97, 97.5, 97.55, 97.6, 97.65, 97.7, 97.75, 97.8, 97.85, 97.9, 97.95, 98, 98.05, 98.1, 98.15, 98.2, 98.25, 98.3, 98.35, 98.4, 98.45, 98.5, 98.55, 98.6, 98.65, 98.7, 98.75, 98.8, 98.85, 98.9, 98.95, 99, 99.05, 99.1, 99.15, 99.2, 99.25, 99.3, 99.35, 99.4, 99.45, 99.5, 99.55, 99.6, 99.65, 99.7, 99.75, 99.8, 99.85, 99.9, 99.91, 99.92, 99.93, 99.94, 99.95, 99.96, 99.97, 99.98, 99.99 or 100% identical to the 16S sequence of Y. entomophaga MH96 (set forth hereirn as SEQ ID NO: 1) and/or a whole genome sequence that is about/at least 95, 95.5, 95.55, 95.6, 95.65, 95.7, 95.75, 95.8, 95.85, 95.9, 95.95, 96, 96.05, 96.1, 96.15, 96.2, 96.25, 96.3, 96.35, 96.4, 96.45, 96.5, 96.55, 96.6, 96.65, 96.7, 96.75, 96.8, 96.85, 96.9, 96.95, 97, 97.5, 97.55, 97.6, 97.65, 97.7, 97.75, 97.8, 97.85, 97.9, 97.95, 98, 98.05, 98.1, 98.15, 98.2, 98.25, 98.3, 98.35, 98.4, 98.45, 98.5, 98.55, 98.6, 98.65, 98.7, 98.75, 98.8, 98.85, 98.9, 98.95, 99, 99.05, 99.1, 99.15, 99.2, 99.25, 99.3, 99.35, 99.4, 99.45, 99.5, 99.55, 99.6, 99.65, 99.7, 99.75, 99.8, 99.85, 99.9 or 99.95% identical to the whole genome sequence of Y. entomophaga MH96), closely related modified microbial strains derived from Y. entomophaga MH96 (e.g., modified microbial strains derived from Y. entomophaga MH96and having a 16S sequence that is about/at least 95, 95.5, 95.55, 95.6, 95.65, 95.7, 95.75, 95.8, 95.85, 95.9, 95.95, 96, 96.05, 96.1, 96.15, 96.2, 96.25, 96.3, 96.35, 96.4, 96.45, 96.5, 96.55, 96.6, 96.65, 96.7, 96.75, 96.8, 96.85, 96.9, 96.95, 97, 97.5, 97.55, 97.6, 97.65, 97.7, 97.75, 97.8, 97.85, 97.9, 97.95, 98, 98.05, 98.1, 98.15, 98.2, 98.25, 98.3, 98.35, 98.4, 98.45, 98.5, 98.55, 98.6, 98.65, 98.7, 98.75, 98.8, 98.85, 98.9, 98.95, 99, 99.05, 99.1, 99.15, 99.2, 99.25, 99.3, 99.35, 99.4, 99.45, 99.5, 99.55, 99.6, 99.65, 99.7, 99.75, 99.8, 99.85, 99.9, 99.91, 99.92, 99.93, 99.94, 99.95, 99.96, 99.97, 99.98, 99.99 or 100% identical to the 16S sequence of Y. entomophaga MH96 (set forth hereirn as SEQ ID NO: 1) and/or a whole genome sequence that is about/at least 95, 95.5, 95.55, 95.6, 95.65, 95.7, 95.75, 95.8, 95.85, 95.9, 95.95, 96, 96.05, 96.1, 96.15, 96.2, 96.25, 96.3, 96.35, 96.4, 96.45, 96.5, 96.55, 96.6, 96.65, 96.7, 96.75, 96.8, 96.85, 96.9, 96.95, 97, 97.5, 97.55, 97.6, 97.65, 97.7, 97.75, 97.8, 97.85, 97.9, 97.95, 98, 98.05, 98.1, 98.15, 98.2, 98.25, 98.3, 98.35, 98.4, 98.45, 98.5, 98.55, 98.6, 98.65, 98.7, 98.75, 98.8, 98.85, 98.9, 98.95, 99, 99.05, 99.1, 99.15, 99.2, 99.25, 99.3, 99.35, 99.4, 99.45, 99.5, 99.55, 99.6, 99.65, 99.7, 99.75, 99.8, 99.85, 99.9 or 99.95% identical to the whole genome sequence of Y. entomophaga MH96), closely related modified microbial strains derived from progeny of Y. entomophaga MH96 (e.g., modified microbial strains derived from one or more progeny of Y. entomophaga MH96 and having a 16S sequence that is about/at least 95, 95.5, 95.55, 95.6, 95.65, 95.7, 95.75, 95.8, 95.85, 95.9, 95.95, 96, 96.05, 96.1, 96.15, 96.2, 96.25, 96.3, 96.35, 96.4, 96.45, 96.5, 96.55, 96.6, 96.65, 96.7, 96.75, 96.8, 96.85, 96.9, 96.95, 97, 97.5, 97.55, 97.6, 97.65, 97.7, 97.75, 97.8, 97.85, 97.9, 97.95, 98, 98.05, 98.1, 98.15, 98.2, 98.25, 98.3, 98.35, 98.4, 98.45, 98.5, 98.55, 98.6, 98.65, 98.7, 98.75, 98.8, 98.85, 98.9, 98.95, 99, 99.05, 99.1, 99.15, 99.2, 99.25, 99.3, 99.35, 99.4, 99.45, 99.5, 99.55, 99.6, 99.65, 99.7, 99.75, 99.8, 99.85, 99.9, 99.91, 99.92, 99.93, 99.94, 99.95, 99.96, 99.97, 99.98, 99.99 or 100% identical to the 16S sequence of Y. entomophaga MH96 (set forth hereirn as SEQ ID NO: 1) and/or a whole genome sequence that is about/at least 95, 95.5, 95.55, 95.6, 95.65, 95.7, 95.75, 95.8, 95.85, 95.9, 95.95, 96, 96.05, 96.1, 96.15, 96.2, 96.25, 96.3, 96.35, 96.4, 96.45, 96.5, 96.55, 96.6, 96.65, 96.7, 96.75, 96.8, 96.85, 96.9, 96.95, 97, 97.5, 97.55, 97.6, 97.65, 97.7, 97.75, 97.8, 97.85, 97.9, 97.95, 98, 98.05, 98.1, 98.15, 98.2, 98.25, 98.3, 98.35, 98.4, 98.45, 98.5, 98.55, 98.6, 98.65, 98.7, 98.75, 98.8, 98.85, 98.9, 98.95, 99, 99.05, 99.1, 99.15, 99.2, 99.25, 99.3, 99.35, 99.4, 99.45, 99.5, 99.55, 99.6, 99.65, 99.7, 99.75, 99.8, 99.85, 99.9 or 99.95% identical to the whole genome sequence of Y. entomophaga MH96), and other closely related strains (e.g., Yersinia strains having a 16S sequence that is about/at least 95, 95.5, 95.55, 95.6, 95.65, 95.7, 95.75, 95.8, 95.85, 95.9, 95.95, 96, 96.05, 96.1, 96.15, 96.2, 96.25, 96.3, 96.35, 96.4, 96.45, 96.5, 96.55, 96.6, 96.65, 96.7, 96.75, 96.8, 96.85, 96.9, 96.95, 97, 97.5, 97.55, 97.6, 97.65, 97.7, 97.75, 97.8, 97.85, 97.9, 97.95, 98, 98.05, 98.1, 98.15, 98.2, 98.25, 98.3, 98.35, 98.4, 98.45, 98.5, 98.55, 98.6, 98.65, 98.7, 98.75, 98.8, 98.85, 98.9, 98.95, 99, 99.05, 99.1, 99.15, 99.2, 99.25, 99.3, 99.35, 99.4, 99.45, 99.5, 99.55, 99.6, 99.65, 99.7, 99.75, 99.8, 99.85, 99.9, 99.91, 99.92, 99.93, 99.94, 99.95, 99.96, 99.97, 99.98, 99.99 or 100% identical to the 16S sequence of Y. entomophaga MH96 (set forth hereirn as SEQ ID NO: 1) and/or a whole genome sequence that is about/at least 95, 95.5, 95.55, 95.6, 95.65, 95.7, 95.75, 95.8, 95.85, 95.9, 95.95, 96, 96.05, 96.1, 96.15, 96.2, 96.25, 96.3, 96.35, 96.4, 96.45, 96.5, 96.55, 96.6, 96.65, 96.7, 96.75, 96.8, 96.85, 96.9, 96.95, 97, 97.5, 97.55, 97.6, 97.65, 97.7, 97.75, 97.8, 97.85, 97.9, 97.95, 98, 98.05, 98.1, 98.15, 98.2, 98.25, 98.3, 98.35, 98.4, 98.45, 98.5, 98.55, 98.6, 98.65, 98.7, 98.75, 98.8, 98.85, 98.9, 98.95, 99, 99.05, 99.1, 99.15, 99.2, 99.25, 99.3, 99.35, 99.4, 99.45, 99.5, 99.55, 99.6, 99.65, 99.7, 99.75, 99.8, 99.85, 99.9 or 99.95% identical to the whole genome sequence of Y. entomophaga MH96), closely related progeny of Y. entomophaga O24G3R, (e.g., progeny having a 16S sequence that is about/at least 95, 95.5, 95.55, 95.6, 95.65, 95.7, 95.75, 95.8, 95.85, 95.9, 95.95, 96, 96.05, 96.1, 96.15, 96.2, 96.25, 96.3, 96.35, 96.4, 96.45, 96.5, 96.55, 96.6, 96.65, 96.7, 96.75, 96.8, 96.85, 96.9, 96.95, 97, 97.5, 97.55, 97.6, 97.65, 97.7, 97.75, 97.8, 97.85, 97.9, 97.95, 98, 98.05, 98.1, 98.15, 98.2, 98.25, 98.3, 98.35, 98.4, 98.45, 98.5, 98.55, 98.6, 98.65, 98.7, 98.75, 98.8, 98.85, 98.9, 98.95, 99, 99.05, 99.1, 99.15, 99.2, 99.25, 99.3, 99.35, 99.4, 99.45, 99.5, 99.55, 99.6, 99.65, 99.7, 99.75, 99.8, 99.85, 99.9, 99.91, 99.92, 99.93, 99.94, 99.95, 99.96, 99.97, 99.98, 99.99 or 100% identical to the 16S sequence of Y. entomophaga O24G3R and/or a whole genome sequence that is about/at least 95, 95.5, 95.55, 95.6, 95.65, 95.7, 95.75, 95.8, 95.85, 95.9, 95.95, 96, 96.05, 96.1, 96.15, 96.2, 96.25, 96.3, 96.35, 96.4, 96.45, 96.5, 96.55, 96.6, 96.65, 96.7, 96.75, 96.8, 96.85, 96.9, 96.95, 97, 97.5, 97.55, 97.6, 97.65, 97.7, 97.75, 97.8, 97.85, 97.9, 97.95, 98, 98.05, 98.1, 98.15, 98.2, 98.25, 98.3, 98.35, 98.4, 98.45, 98.5, 98.55, 98.6, 98.65, 98.7, 98.75, 98.8, 98.85, 98.9, 98.95, 99, 99.05, 99.1, 99.15, 99.2, 99.25, 99.3, 99.35, 99.4, 99.45, 99.5, 99.55, 99.6, 99.65, 99.7, 99.75, 99.8, 99.85, 99.9 or 99.95% identical to the whole genome sequence of Y. entomophaga O24G3R), closely related modified microbial strains derived from Y. entomophaga O24G3R (e.g., modified microbial strains derived from Y. entomophaga O24G3R and having a 16S sequence that is about/at least 95, 95.5, 95.55, 95.6, 95.65, 95.7, 95.75, 95.8, 95.85, 95.9, 95.95, 96, 96.05, 96.1, 96.15, 96.2, 96.25, 96.3, 96.35, 96.4, 96.45, 96.5, 96.55, 96.6, 96.65, 96.7, 96.75, 96.8, 96.85, 96.9, 96.95, 97, 97.5, 97.55, 97.6, 97.65, 97.7, 97.75, 97.8, 97.85, 97.9, 97.95, 98, 98.05, 98.1, 98.15, 98.2, 98.25, 98.3, 98.35, 98.4, 98.45, 98.5, 98.55, 98.6, 98.65, 98.7, 98.75, 98.8, 98.85, 98.9, 98.95, 99, 99.05, 99.1, 99.15, 99.2, 99.25, 99.3, 99.35, 99.4, 99.45, 99.5, 99.55, 99.6, 99.65, 99.7, 99.75, 99.8, 99.85, 99.9, 99.91, 99.92, 99.93, 99.94, 99.95, 99.96, 99.97, 99.98, 99.99 or 100% identical to the 16S sequence of Y. entomophaga O24G3R and/or a whole genome sequence that is about/at least 95, 95.5, 95.55, 95.6, 95.65, 95.7, 95.75, 95.8, 95.85, 95.9, 95.95, 96, 96.05, 96.1, 96.15, 96.2, 96.25, 96.3, 96.35, 96.4, 96.45, 96.5, 96.55, 96.6, 96.65, 96.7, 96.75, 96.8, 96.85, 96.9, 96.95, 97, 97.5, 97.55, 97.6, 97.65, 97.7, 97.75, 97.8, 97.85, 97.9, 97.95, 98, 98.05, 98.1, 98.15, 98.2, 98.25, 98.3, 98.35, 98.4, 98.45, 98.5, 98.55, 98.6, 98.65, 98.7, 98.75, 98.8, 98.85, 98.9, 98.95, 99, 99.05, 99.1, 99.15, 99.2, 99.25, 99.3, 99.35, 99.4, 99.45, 99.5, 99.55, 99.6, 99.65, 99.7, 99.75, 99.8, 99.85, 99.9 or 99.95% identical to the whole genome sequence of Y. entomophaga O24G3R), closely related modified microbial strains derived from progeny of Y. entomophaga MH96 (e.g., modified microbial strains derived from one or more progeny of Y. entomophaga O24G3R and having a 16S sequence that is about/at least 95, 95.5, 95.55, 95.6, 95.65, 95.7, 95.75, 95.8, 95.85, 95.9, 95.95, 96, 96.05, 96.1, 96.15, 96.2, 96.25, 96.3, 96.35, 96.4, 96.45, 96.5, 96.55, 96.6, 96.65, 96.7, 96.75, 96.8, 96.85, 96.9, 96.95, 97, 97.5, 97.55, 97.6, 97.65, 97.7, 97.75, 97.8, 97.85, 97.9, 97.95, 98, 98.05, 98.1, 98.15, 98.2, 98.25, 98.3, 98.35, 98.4, 98.45, 98.5, 98.55, 98.6, 98.65, 98.7, 98.75, 98.8, 98.85, 98.9, 98.95, 99, 99.05, 99.1, 99.15, 99.2, 99.25, 99.3, 99.35, 99.4, 99.45, 99.5, 99.55, 99.6, 99.65, 99.7, 99.75, 99.8, 99.85, 99.9, 99.91, 99.92, 99.93, 99.94, 99.95, 99.96, 99.97, 99.98, 99.99 or 100% identical to the 16S sequence of Y. entomophaga O24G3R and/or a whole genome sequence that is about/at least 95, 95.5, 95.55, 95.6, 95.65, 95.7, 95.75, 95.8, 95.85, 95.9, 95.95, 96, 96.05, 96.1, 96.15, 96.2, 96.25, 96.3, 96.35, 96.4, 96.45, 96.5, 96.55, 96.6, 96.65, 96.7, 96.75, 96.8, 96.85, 96.9, 96.95, 97, 97.5, 97.55, 97.6, 97.65, 97.7, 97.75, 97.8, 97.85, 97.9, 97.95, 98, 98.05, 98.1, 98.15, 98.2, 98.25, 98.3, 98.35, 98.4, 98.45, 98.5, 98.55, 98.6, 98.65, 98.7, 98.75, 98.8, 98.85, 98.9, 98.95, 99, 99.05, 99.1, 99.15, 99.2, 99.25, 99.3, 99.35, 99.4, 99.45, 99.5, 99.55, 99.6, 99.65, 99.7, 99.75, 99.8, 99.85, 99.9 or 99.95% identical to the whole genome sequence of Y. entomophaga O24G3R), and other closely related strains (e.g., Yersinia strains having a 16S sequence that is about/at least 95, 95.5, 95.55, 95.6, 95.65, 95.7, 95.75, 95.8, 95.85, 95.9, 95.95, 96, 96.05, 96.1, 96.15, 96.2, 96.25, 96.3, 96.35, 96.4, 96.45, 96.5, 96.55, 96.6, 96.65, 96.7, 96.75, 96.8, 96.85, 96.9, 96.95, 97, 97.5, 97.55, 97.6, 97.65, 97.7, 97.75, 97.8, 97.85, 97.9, 97.95, 98, 98.05, 98.1, 98.15, 98.2, 98.25, 98.3, 98.35, 98.4, 98.45, 98.5, 98.55, 98.6, 98.65, 98.7, 98.75, 98.8, 98.85, 98.9, 98.95, 99, 99.05, 99.1, 99.15, 99.2, 99.25, 99.3, 99.35, 99.4, 99.45, 99.5, 99.55, 99.6, 99.65, 99.7, 99.75, 99.8, 99.85, 99.9, 99.91, 99.92, 99.93, 99.94, 99.95, 99.96, 99.97, 99.98, 99.99 or 100% identical to the 16S sequence of Y. entomophaga O24G3R and/or a whole genome sequence that is about/at least 95, 95.5, 95.55, 95.6, 95.65, 95.7, 95.75, 95.8, 95.85, 95.9, 95.95, 96, 96.05, 96.1, 96.15, 96.2, 96.25, 96.3, 96.35, 96.4, 96.45, 96.5, 96.55, 96.6, 96.65, 96.7, 96.75, 96.8, 96.85, 96.9, 96.95, 97, 97.5, 97.55, 97.6, 97.65, 97.7, 97.75, 97.8, 97.85, 97.9, 97.95, 98, 98.05, 98.1, 98.15, 98.2, 98.25, 98.3, 98.35, 98.4, 98.45, 98.5, 98.55, 98.6, 98.65, 98.7, 98.75, 98.8, 98.85, 98.9, 98.95, 99, 99.05, 99.1, 99.15, 99.2, 99.25, 99.3, 99.35, 99.4, 99.45, 99.5, 99.55, 99.6, 99.65, 99.7, 99.75, 99.8, 99.85, 99.9 or 99.95% identical to the whole genome sequence of Y. entomophaga O24G3R), closely related progeny of Y. entomophaga O24KEK, (e.g., progeny having a 16S sequence that is about/at least 95, 95.5, 95.55, 95.6, 95.65, 95.7, 95.75, 95.8, 95.85, 95.9, 95.95, 96, 96.05, 96.1, 96.15, 96.2, 96.25, 96.3, 96.35, 96.4, 96.45, 96.5, 96.55, 96.6, 96.65, 96.7, 96.75, 96.8, 96.85, 96.9, 96.95, 97, 97.5, 97.55, 97.6, 97.65, 97.7, 97.75, 97.8, 97.85, 97.9, 97.95, 98, 98.05, 98.1, 98.15, 98.2, 98.25, 98.3, 98.35, 98.4, 98.45, 98.5, 98.55, 98.6, 98.65, 98.7, 98.75, 98.8, 98.85, 98.9, 98.95, 99, 99.05, 99.1, 99.15, 99.2, 99.25, 99.3, 99.35, 99.4, 99.45, 99.5, 99.55, 99.6, 99.65, 99.7, 99.75, 99.8, 99.85, 99.9, 99.91, 99.92, 99.93, 99.94, 99.95, 99.96, 99.97, 99.98, 99.99 or 100% identical to the 16S sequence of Y. entomophaga O24KEK and/or a whole genome sequence that is about/at least 95, 95.5, 95.55, 95.6, 95.65, 95.7, 95.75, 95.8, 95.85, 95.9, 95.95, 96, 96.05, 96.1, 96.15, 96.2, 96.25, 96.3, 96.35, 96.4, 96.45, 96.5, 96.55, 96.6, 96.65, 96.7, 96.75, 96.8, 96.85, 96.9, 96.95, 97, 97.5, 97.55, 97.6, 97.65, 97.7, 97.75, 97.8, 97.85, 97.9, 97.95, 98, 98.05, 98.1, 98.15, 98.2, 98.25, 98.3, 98.35, 98.4, 98.45, 98.5, 98.55, 98.6, 98.65, 98.7, 98.75, 98.8, 98.85, 98.9, 98.95, 99, 99.05, 99.1, 99.15, 99.2, 99.25, 99.3, 99.35, 99.4, 99.45, 99.5, 99.55, 99.6, 99.65, 99.7, 99.75, 99.8, 99.85, 99.9 or 99.95% identical to the whole genome sequence of Y. entomophaga O24KEK), closely related modified microbial strains derived from Y. entomophaga O24KEK (e.g., modified microbial strains derived from Y. entomophaga O24KEK and having a 16S sequence that is about/at least 95, 95.5, 95.55, 95.6, 95.65, 95.7, 95.75, 95.8, 95.85, 95.9, 95.95, 96, 96.05, 96.1, 96.15, 96.2, 96.25, 96.3, 96.35, 96.4, 96.45, 96.5, 96.55, 96.6, 96.65, 96.7, 96.75, 96.8, 96.85, 96.9, 96.95, 97, 97.5, 97.55, 97.6, 97.65, 97.7, 97.75, 97.8, 97.85, 97.9, 97.95, 98, 98.05, 98.1, 98.15, 98.2, 98.25, 98.3, 98.35, 98.4, 98.45, 98.5, 98.55, 98.6, 98.65, 98.7, 98.75, 98.8, 98.85, 98.9, 98.95, 99, 99.05, 99.1, 99.15, 99.2, 99.25, 99.3, 99.35, 99.4, 99.45, 99.5, 99.55, 99.6, 99.65, 99.7, 99.75, 99.8, 99.85, 99.9, 99.91, 99.92, 99.93, 99.94, 99.95, 99.96, 99.97, 99.98, 99.99 or 100% identical to the 16S sequence of Y. entomophaga O24KEK and/or a whole genome sequence that is about/at least 95, 95.5, 95.55, 95.6, 95.65, 95.7, 95.75, 95.8, 95.85, 95.9, 95.95, 96, 96.05, 96.1, 96.15, 96.2, 96.25, 96.3, 96.35, 96.4, 96.45, 96.5, 96.55, 96.6, 96.65, 96.7, 96.75, 96.8, 96.85, 96.9, 96.95, 97, 97.5, 97.55, 97.6, 97.65, 97.7, 97.75, 97.8, 97.85, 97.9, 97.95, 98, 98.05, 98.1, 98.15, 98.2, 98.25, 98.3, 98.35, 98.4, 98.45, 98.5, 98.55, 98.6, 98.65, 98.7, 98.75, 98.8, 98.85, 98.9, 98.95, 99, 99.05, 99.1, 99.15, 99.2, 99.25, 99.3, 99.35, 99.4, 99.45, 99.5, 99.55, 99.6, 99.65, 99.7, 99.75, 99.8, 99.85, 99.9 or 99.95% identical to the whole genome sequence of Y. entomophaga O24KEK), closely related modified microbial strains derived from progeny of Y. entomophaga O24KEK (e.g., modified microbial strains derived from one or more progeny of Y. entomophaga O24KEK and having a 16S sequence that is about/at least 95, 95.5, 95.55, 95.6, 95.65, 95.7, 95.75, 95.8, 95.85, 95.9, 95.95, 96, 96.05, 96.1, 96.15, 96.2, 96.25, 96.3, 96.35, 96.4, 96.45, 96.5, 96.55, 96.6, 96.65, 96.7, 96.75, 96.8, 96.85, 96.9, 96.95, 97, 97.5, 97.55, 97.6, 97.65, 97.7, 97.75, 97.8, 97.85, 97.9, 97.95, 98, 98.05, 98.1, 98.15, 98.2, 98.25, 98.3, 98.35, 98.4, 98.45, 98.5, 98.55, 98.6, 98.65, 98.7, 98.75, 98.8, 98.85, 98.9, 98.95, 99, 99.05, 99.1, 99.15, 99.2, 99.25, 99.3, 99.35, 99.4, 99.45, 99.5, 99.55, 99.6, 99.65, 99.7, 99.75, 99.8, 99.85, 99.9, 99.91, 99.92, 99.93, 99.94, 99.95, 99.96, 99.97, 99.98, 99.99 or 100% identical to the 16S sequence of Y. entomophaga O24KEK and/or a whole genome sequence that is about/at least 95, 95.5, 95.55, 95.6, 95.65, 95.7, 95.75, 95.8, 95.85, 95.9, 95.95, 96, 96.05, 96.1, 96.15, 96.2, 96.25, 96.3, 96.35, 96.4, 96.45, 96.5, 96.55, 96.6, 96.65, 96.7, 96.75, 96.8, 96.85, 96.9, 96.95, 97, 97.5, 97.55, 97.6, 97.65, 97.7, 97.75, 97.8, 97.85, 97.9, 97.95, 98, 98.05, 98.1, 98.15, 98.2, 98.25, 98.3, 98.35, 98.4, 98.45, 98.5, 98.55, 98.6, 98.65, 98.7, 98.75, 98.8, 98.85, 98.9, 98.95, 99, 99.05, 99.1, 99.15, 99.2, 99.25, 99.3, 99.35, 99.4, 99.45, 99.5, 99.55, 99.6, 99.65, 99.7, 99.75, 99.8, 99.85, 99.9 or 99.95% identical to the whole genome sequence of Y. entomophaga O24KEK), and other closely related strains (e.g., Yersinia strains having a 16S sequence that is about/at least 95, 95.5, 95.55, 95.6, 95.65, 95.7, 95.75, 95.8, 95.85, 95.9, 95.95, 96, 96.05, 96.1, 96.15, 96.2, 96.25, 96.3, 96.35, 96.4, 96.45, 96.5, 96.55, 96.6, 96.65, 96.7, 96.75, 96.8, 96.85, 96.9, 96.95, 97, 97.5, 97.55, 97.6, 97.65, 97.7, 97.75, 97.8, 97.85, 97.9, 97.95, 98, 98.05, 98.1, 98.15, 98.2, 98.25, 98.3, 98.35, 98.4, 98.45, 98.5, 98.55, 98.6, 98.65, 98.7, 98.75, 98.8, 98.85, 98.9, 98.95, 99, 99.05, 99.1, 99.15, 99.2, 99.25, 99.3, 99.35, 99.4, 99.45, 99.5, 99.55, 99.6, 99.65, 99.7, 99.75, 99.8, 99.85, 99.9, 99.91, 99.92, 99.93, 99.94, 99.95, 99.96, 99.97, 99.98, 99.99 or 100% identical to the 16S sequence of Y. entomophaga O24KEK and/or a whole genome sequence that is about/at least 95, 95.5, 95.55, 95.6, 95.65, 95.7, 95.75, 95.8, 95.85, 95.9, 95.95, 96, 96.05, 96.1, 96.15, 96.2, 96.25, 96.3, 96.35, 96.4, 96.45, 96.5, 96.55, 96.6, 96.65, 96.7, 96.75, 96.8, 96.85, 96.9, 96.95, 97, 97.5, 97.55, 97.6, 97.65, 97.7, 97.75, 97.8, 97.85, 97.9, 97.95, 98, 98.05, 98.1, 98.15, 98.2, 98.25, 98.3, 98.35, 98.4, 98.45, 98.5, 98.55, 98.6, 98.65, 98.7, 98.75, 98.8, 98.85, 98.9, 98.95, 99, 99.05, 99.1, 99.15, 99.2, 99.25, 99.3, 99.35, 99.4, 99.45, 99.5, 99.55, 99.6, 99.65, 99.7, 99.75, 99.8, 99.85, 99.9 or 99.95% identical to the whole genome sequence of Y. entomophaga O24KEK), closely related progeny of Y. entomophaga O333A4, (e.g., progeny having a 16S sequence that is about/at least 95, 95.5, 95.55, 95.6, 95.65, 95.7, 95.75, 95.8, 95.85, 95.9, 95.95, 96, 96.05, 96.1, 96.15, 96.2, 96.25, 96.3, 96.35, 96.4, 96.45, 96.5, 96.55, 96.6, 96.65, 96.7, 96.75, 96.8, 96.85, 96.9, 96.95, 97, 97.5, 97.55, 97.6, 97.65, 97.7, 97.75, 97.8, 97.85, 97.9, 97.95, 98, 98.05, 98.1, 98.15, 98.2, 98.25, 98.3, 98.35, 98.4, 98.45, 98.5, 98.55, 98.6, 98.65, 98.7, 98.75, 98.8, 98.85, 98.9, 98.95, 99, 99.05, 99.1, 99.15, 99.2, 99.25, 99.3, 99.35, 99.4, 99.45, 99.5, 99.55, 99.6, 99.65, 99.7, 99.75, 99.8, 99.85, 99.9, 99.91, 99.92, 99.93, 99.94, 99.95, 99.96, 99.97, 99.98, 99.99 or 100% identical to the 16S sequence of Y. entomophaga O333A4 and/or a whole genome sequence that is about/at least 95, 95.5, 95.55, 95.6, 95.65, 95.7, 95.75, 95.8, 95.85, 95.9, 95.95, 96, 96.05, 96.1, 96.15, 96.2, 96.25, 96.3, 96.35, 96.4, 96.45, 96.5, 96.55, 96.6, 96.65, 96.7, 96.75, 96.8, 96.85, 96.9, 96.95, 97, 97.5, 97.55, 97.6, 97.65, 97.7, 97.75, 97.8, 97.85, 97.9, 97.95, 98, 98.05, 98.1, 98.15, 98.2, 98.25, 98.3, 98.35, 98.4, 98.45, 98.5, 98.55, 98.6, 98.65, 98.7, 98.75, 98.8, 98.85, 98.9, 98.95, 99, 99.05, 99.1, 99.15, 99.2, 99.25, 99.3, 99.35, 99.4, 99.45, 99.5, 99.55, 99.6, 99.65, 99.7, 99.75, 99.8, 99.85, 99.9 or 99.95% identical to the whole genome sequence of Y. entomophaga O333A4), closely related modified microbial strains derived from Y. entomophaga O333A4 (e.g., modified microbial strains derived from Y. entomophaga O333A4 and having a 16S sequence that is about/at least 95, 95.5, 95.55, 95.6, 95.65, 95.7, 95.75, 95.8, 95.85, 95.9, 95.95, 96, 96.05, 96.1, 96.15, 96.2, 96.25, 96.3, 96.35, 96.4, 96.45, 96.5, 96.55, 96.6, 96.65, 96.7, 96.75, 96.8, 96.85, 96.9, 96.95, 97, 97.5, 97.55, 97.6, 97.65, 97.7, 97.75, 97.8, 97.85, 97.9, 97.95, 98, 98.05, 98.1, 98.15, 98.2, 98.25, 98.3, 98.35, 98.4, 98.45, 98.5, 98.55, 98.6, 98.65, 98.7, 98.75, 98.8, 98.85, 98.9, 98.95, 99, 99.05, 99.1, 99.15, 99.2, 99.25, 99.3, 99.35, 99.4, 99.45, 99.5, 99.55, 99.6, 99.65, 99.7, 99.75, 99.8, 99.85, 99.9, 99.91, 99.92, 99.93, 99.94, 99.95, 99.96, 99.97, 99.98, 99.99 or 100% identical to the 16S sequence of Y. entomophaga O333A4 and/or a whole genome sequence that is about/at least 95, 95.5, 95.55, 95.6, 95.65, 95.7, 95.75, 95.8, 95.85, 95.9, 95.95, 96, 96.05, 96.1, 96.15, 96.2, 96.25, 96.3, 96.35, 96.4, 96.45, 96.5, 96.55, 96.6, 96.65, 96.7, 96.75, 96.8, 96.85, 96.9, 96.95, 97, 97.5, 97.55, 97.6, 97.65, 97.7, 97.75, 97.8, 97.85, 97.9, 97.95, 98, 98.05, 98.1, 98.15, 98.2, 98.25, 98.3, 98.35, 98.4, 98.45, 98.5, 98.55, 98.6, 98.65, 98.7, 98.75, 98.8, 98.85, 98.9, 98.95, 99, 99.05, 99.1, 99.15, 99.2, 99.25, 99.3, 99.35, 99.4, 99.45, 99.5, 99.55, 99.6, 99.65, 99.7, 99.75, 99.8, 99.85, 99.9 or 99.95% identical to the whole genome sequence of Y. entomophaga O333A4), closely related modified microbial strains derived from progeny of Y. entomophaga O333A4 (e.g., modified microbial strains derived from one or more progeny of Y. entomophaga O333A4 and having a 16S sequence that is about/at least 95, 95.5, 95.55, 95.6, 95.65, 95.7, 95.75, 95.8, 95.85, 95.9, 95.95, 96, 96.05, 96.1, 96.15, 96.2, 96.25, 96.3, 96.35, 96.4, 96.45, 96.5, 96.55, 96.6, 96.65, 96.7, 96.75, 96.8, 96.85, 96.9, 96.95, 97, 97.5, 97.55, 97.6, 97.65, 97.7, 97.75, 97.8, 97.85, 97.9, 97.95, 98, 98.05, 98.1, 98.15, 98.2, 98.25, 98.3, 98.35, 98.4, 98.45, 98.5, 98.55, 98.6, 98.65, 98.7, 98.75, 98.8, 98.85, 98.9, 98.95, 99, 99.05, 99.1, 99.15, 99.2, 99.25, 99.3, 99.35, 99.4, 99.45, 99.5, 99.55, 99.6, 99.65, 99.7, 99.75, 99.8, 99.85, 99.9, 99.91, 99.92, 99.93, 99.94, 99.95, 99.96, 99.97, 99.98, 99.99 or 100% identical to the 16S sequence of Y. entomophaga O333A4 and/or a whole genome sequence that is about/at least 95, 95.5, 95.55, 95.6, 95.65, 95.7, 95.75, 95.8, 95.85, 95.9, 95.95, 96, 96.05, 96.1, 96.15, 96.2, 96.25, 96.3, 96.35, 96.4, 96.45, 96.5, 96.55, 96.6, 96.65, 96.7, 96.75, 96.8, 96.85, 96.9, 96.95, 97, 97.5, 97.55, 97.6, 97.65, 97.7, 97.75, 97.8, 97.85, 97.9, 97.95, 98, 98.05, 98.1, 98.15, 98.2, 98.25, 98.3, 98.35, 98.4, 98.45, 98.5, 98.55, 98.6, 98.65, 98.7, 98.75, 98.8, 98.85, 98.9, 98.95, 99, 99.05, 99.1, 99.15, 99.2, 99.25, 99.3, 99.35, 99.4, 99.45, 99.5, 99.55, 99.6, 99.65, 99.7, 99.75, 99.8, 99.85, 99.9 or 99.95% identical to the whole genome sequence of Y. entomophaga O333A4), and other closely related strains (e.g., Yersinia strains having a 16S sequence that is about/at least 95, 95.5, 95.55, 95.6, 95.65, 95.7, 95.75, 95.8, 95.85, 95.9, 95.95, 96, 96.05, 96.1, 96.15, 96.2, 96.25, 96.3, 96.35, 96.4, 96.45, 96.5, 96.55, 96.6, 96.65, 96.7, 96.75, 96.8, 96.85, 96.9, 96.95, 97, 97.5, 97.55, 97.6, 97.65, 97.7, 97.75, 97.8, 97.85, 97.9, 97.95, 98, 98.05, 98.1, 98.15, 98.2, 98.25, 98.3, 98.35, 98.4, 98.45, 98.5, 98.55, 98.6, 98.65, 98.7, 98.75, 98.8, 98.85, 98.9, 98.95, 99, 99.05, 99.1, 99.15, 99.2, 99.25, 99.3, 99.35, 99.4, 99.45, 99.5, 99.55, 99.6, 99.65, 99.7, 99.75, 99.8, 99.85, 99.9, 99.91, 99.92, 99.93, 99.94, 99.95, 99.96, 99.97, 99.98, 99.99 or 100% identical to the 16S sequence of Y. entomophaga O333A4 and/or a whole genome sequence that is about/at least 95, 95.5, 95.55, 95.6, 95.65, 95.7, 95.75, 95.8, 95.85, 95.9, 95.95, 96, 96.05, 96.1, 96.15, 96.2, 96.25, 96.3, 96.35, 96.4, 96.45, 96.5, 96.55, 96.6, 96.65, 96.7, 96.75, 96.8, 96.85, 96.9, 96.95, 97, 97.5, 97.55, 97.6, 97.65, 97.7, 97.75, 97.8, 97.85, 97.9, 97.95, 98, 98.05, 98.1, 98.15, 98.2, 98.25, 98.3, 98.35, 98.4, 98.45, 98.5, 98.55, 98.6, 98.65, 98.7, 98.75, 98.8, 98.85, 98.9, 98.95, 99, 99.05, 99.1, 99.15, 99.2, 99.25, 99.3, 99.35, 99.4, 99.45, 99.5, 99.55, 99.6, 99.65, 99.7, 99.75, 99.8, 99.85, 99.9 or 99.95% identical to the whole genome sequence of Y. entomophaga O333A4), which may themselves exhibit insecticidal or other pesticidal activities and be useful for protecting and/or enhancing the growth and/or yield of various plants, including, but not limited to, cereals and pseudocereals, such as barley, buckwheat, corn, millet, oats, quinoa, rice, lye, sorghum and wheat, and legumes, such as alfalfa, beans, carob, clover, guar, lentils, mesquite, peas, peanuts, soybeans, tamarind, tragacanth and vetch.

Thus, it is to be understood that the present disclosure encompasses isolated microbial strains, biologically pure cultures, inoculant compostions, non-naturally occurring compositions, plants, plant parts, processed products, crops, kits, methods and uses, such as those set forth herein, in which one or more closely related progeny of Y. entomophaga MH96, Y. entomophaga O24G3R, Y. entomophaga O24KEK, Y. entomophaga O333A4, Y. entomophaga O23ZMJ, Y. entomophaga O348UX and/or Y. entomophaga O33ZDX, one or more closely related modified microbial strains derived from Y. entomophaga MH96, Y. entomophaga O24G3R, Y. entomophaga O24KEK, Y. entomophaga O333A4, Y. entomophaga O23ZMJ, Y. entomophaga O348UX and/or Y. entomophaga O33ZDX, one or more closely related modified microbial strains derived from progeny of Y. entomophaga MH96, Y. entomophaga O24G3R, Y. entomophaga O24KEK, Y. entomophaga O333A4, Y. entomophaga O23ZMJ, Y. entomophaga O348UX and/or Y. entomophaga O33ZDX, and/or one or more other close relatives of Y. entomophaga MH96, Y. entomophaga O24G3R, Y. entomophaga O24KEK, Y. entomophaga O333A4, Y. entomophaga O23ZMJ, Y. entomophaga O348UX and/or Y. entomophaga O33ZDX is/are substituted for Y. entomophaga MH96, Y. entomophaga O24G3R, Y. entomophaga O24KEK and/or Y. entomophaga O333A4.

While certain aspects of the present disclosure will hereinafter be described with reference to embodiments thereof, it will be understood by those of ordinary skill in the art that various changes in form and details may be made therein without departing from the spirit and scope of the present disclosure as defined by the claims.

All publications, patent applications, patents and other references mentioned herein are incorporated by reference in their entirety, except insofar as they contradict any disclosure expressly set forth herein.

The present disclosure describes uses, methods and compositions wherein one or more Yersinia entomophaga (and/or one or more toxins or other components derived therefrom) and/or one or more Yersinia nurmii (and/or one or more toxins or other components derived therefrom) are combined with one or more Bacillus thuringiensis (and/or one or more toxins or other components derived therefrom).

As will be understood by those skilled in the art, it is possible to achieve such combinations in myriad ways. In some embodiments, the desired effect is achieved using a first composition comprising one or more Yersinia entomophaga (and/or one or more toxins or other components derived therefrom) and/or one or more Yersinia nurmii (and/or one or more toxins or other components derived therefrom) and a second compositions comprising one or more Bacillus thuringiensis (and/or one or more toxins or other components derived therefrom). In some embodiments, the desired effect is achieved using a single composition comprising one or more Yersinia entomophaga (and/or one or more toxins or other components derived therefrom) and/or one or more Yersinia nurmii (and/or one or more toxins or other components derived therefrom) as well as one or more Bacillus thuringiensis (and/or one or more toxins or other components derived therefrom). In some embodiments, the desired effect is achieved using a transgenic microorganism (e.g., a Yersinia entomophaga strain that has been modified to express one or more Bacillus thuringiensis toxins, a Yersinia nurmii strain that has been modified to express one or more Bacillus thuringiensis toxins, a Bacillus thuringiensis strain that has been modified to express one or more Yersinia entomophaga toxins and/or one or more Yersinia nurmii toxins). In some embodiments, the desired effect is achieved using a transgenic plant that expresses both Yersinia components and Bacillus thuringiensis components (e.g., a plant that has been modified to express one or more Yersinia entomophaga and/or Yersinia nurmii toxins as well as one or more Bacillus thuringiensis toxins). In some embodiments, the desired effect is achieved using a transgenic plant that expresses one or more Yersinia components and is treated with one or more Bacillus thuringiensis components (e.g. a plant that has been modified to express one or more Yersinia entomophaga toxins and is treated with one or more Bacillus thuringiensis (and/or one or more toxins or other components derived therefrom)). In some embodiments, the desired effect is achieved using a transgenic plant that expresses one or more Bacillus thuringiensis components and is treated with one or more Yersinia components (e.g. a plant that has been modified to express one or more Bacillus thuringiensis toxins and is treated with one or more Yersinia entomophaga (and/or one or more toxins or other components derived therefrom)). It is to be understood that the genes/proteins expressed in such transgenic microorganisms/plants may be variants of the naturally occurring genes/proteins.

Uses, methods and compositions of the present disclosure may comprise/utilize any suitable Yersinia entomophaga and/or Yersinia nurmii, including, but not limited to, those set forth herein as “Yersinia strains of the present disclosure.”

Yersinia entomophaga is a Gram-negative, pesticidal bacterium with activity against a wide range of insects. See, e.g., WO 2007/142543; WO 2008/041863; Hurst et al., INT. J. SYST. EVOL. MICROBIOL. 61:844-849 (2011). Yersinia entomophaga express a toxin complex (TC) called Yen-TC. Yen-TC is reportedly composed of seven subunits—YenA1 and YenA2 and the chitinases Chi1 and Chi2 reportedly form a pentameric cage, into which YenB, and one of YenC1 or YenC2, bind to form the active Yen-TC. Busby et al., J. MOL. BIOL. 415:359-371 (2012). Genes encoding other putative toxins have been identified in Yersinia entomophaga. See, e.g., Hurst et al., TOXINS 8:143 (2016). Toxins and other components may be found in and isolated from the supernatants of media in which Yersinia entomophaga and/or Yersinia nurmii has been cultured. Thus, in some embodiments of the present disclosure, toxins and other Yersinia components are present in (and applied as part of) a cell-free Yersinia culture filtrate. In other embodiments, toxins and other Yersinia components are present (and applied as) isolate molecules that are substantially purified from the cells and culture media from which they are derived.

The type strain of Yersinia entomophaga is MH96, also called MH96T, and earlier called MH-1 or SpK. Y. entomophaga MH96 has been deposited in the Leibniz Institute DSMZ-German Collection of Microorganisms and Cell Cultures as DSM 22339, in the American Type Culture Collection as ATCC BAA-1678, and in the U.S. Department of Agriculture's Agricultural Research Service Culture Collection as NRRL B-67598. The genome sequence of MH96 was published by Hurst, M. R. H. et al., TOXINS 8:143 (2016). The 16S rRNA sequence of MH96 is designated as GenBank Accession No. DQ400782 and is set forth herein as SEQ ID NO: 1.

Yersinia entomophaga (strain MH96 is exemplary) is related to Yersinia nurmii. The combinations of Yersinia and other substances, described herein, may include both Yersinia entomophaga and Yersinia nurmii. Herein, the use of the word Yersinia alone generally is meant to include both Yersinia entomophaga and Yersinia nurmii.

Yersinia entomphaga and Yersinia nurmii are taxonomically distant from other Yersinia, including Yersinia that are pathogenic for humans Yersinia entomphaga and Yersinia nurmii form a distinct clade away from other Yersinia species (Reuter, S. et al., PNAS 111:6768-6773 (2014)). Bioinformatic analysis of the Yersinia entomphaga genome failed to identify orthologs of know Yersinia pestis or Yersinia pseudotuberculosis virulence determinants, for example (Hurst, M. R. H. et al., TOXINS 8:143 (2016)).

Yersinia may be cultured using any suitable method(s), including, but not limited to, liquid-state fermentation and solid-state fermentation. In some examples, Yersinia entomophaga may be grown on/in Luria (LB) agar/medium. The compositions described herein may contain Yersinia organisms and/or may contain a toxin from the organisms (cell free filtrate). The toxin may be purified or partially purified away from other, non-toxin components. Purification or partial purification of the toxin and/or subunits may use standard protein purification methodologies that are known in the art.

Yersinia may be harvested during any suitable growth phase. In some embodiments, Yersinia is allowed to reach the stationary growth phase and is harvested as vegetative cells.

Yersinia may be harvested and/or concentrated using any suitable method(s), including, but not limited to, centrifugation (e.g., density gradient centrifugation, disc stack centrifugation, tubular bowl centrifugation), coagulation, decanting, felt bed collection, filtration (e.g., drum filtration, sieving, ultrafiltration), flocculation, impaction and trapping (e.g., cyclone spore trapping, liquid impingement).

The present disclosure also provides cultures comprising, consisting essentially of or consisting of the Yersinia disclosed herein. In some embodiments, at least 95, 96, 97, 98, 99, 99.5, 99.6, 99.7, 99.8, or 99.9% of subcultures taken from the culture exhibit a genotype that is at least 95, 96, 97, 98, 99%, 99.1%, 99.2%, 99.3%, 99.4%, 99.5%, 99.55%, 99.6%, 99.65%, 99.7%, 99.75%, 99.8%, 99.85%, 99.9%, 99.95%, or 100% identical to that of the Yersinia disclosed herein. In some embodiments, the culture is a biologically pure culture of the Yersinia. These may be combined with similar cultures of Bacillus thuringiensis.

Yersinia may be incorporated into compositions in any suitable amount/concentration. The absolute value of the amount/concentration that is/are sufficient to cause the desired effect(s) may be affected by factors such as the type, size and volume of material to which the composition will be applied and storage conditions (e.g., temperature, relative humidity, duration). Those skilled in the art will understand how to select an effective amount/concentration using routine dose-response experiments.

In some embodiments, compositions of the present disclosure comprise Yersinia in an amount ranging from about 1×10¹ to about 1×10¹², optionally about 1×10³ to about 1×10⁷, colony-forming units (CFU) per gram and/or milliliter of composition. For example, compositions of the present disclosure may comprise about 1×10¹, 1×10², 1×10³, 1×10⁴, 1×10⁵, 1×10⁶, 1×10⁷, 1×10⁸, 1×10⁹, 1×10¹⁰, 1×10¹¹, 1×10¹² or more CFU of Yersinia per gram and/or milliliter of composition. In some embodiments, compositions of the present disclosure comprise at least 1×10⁴, 1×10⁵, 1×10⁶, 1×10⁷, 1×10⁸, 1×10⁹, 1×10¹⁰, 1×10¹¹, 1×10¹² CFU of Yersinia per gram and/or milliliter of composition.

In some embodiments, Yersinia comprises about 0.00000001 to about 95% (by weight) of the composition. In some examples, compositions of the present disclosure may comprise about 1×10⁻¹⁵, 1×10⁻¹⁴, 1×10⁻¹³, 1×10⁻¹², 1×10⁻¹¹, 1×10⁻¹⁰, 1×10⁻⁹, 1×10⁻⁸, 1×10⁻⁷, 1×10⁻⁶, 1×10⁻⁵, 1×10⁻⁴, 1×10⁻³, 1×10⁻², 1×10⁻¹, 0.2, 0.3, 0.4, 0.5, 0.6, 0.7, 0.8, 0.9, 1, 1.25, 1.5, 1.75, 2, 2.25, 2.5, 2.75, 3, 3.25, 3.5, 3.75, 4, 4.25, 4.5, 4.75, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 55, 60, 65, 70, 75, 80, 85, 90, 95% or more (by weight) of Yersinia. In some embodiments, Yersinia comprises about 1 to about 25%, about 5 to about 20%, about 5 to about 15%, about 5 to about 10% or about 8 to about 12% (by weight) of the composition.

In some embodiments, compositions of the present disclosure comprise Yersinia in an effective amount/concentration for enhancing plant growth/yield and/or for insecticidal or other pesticidal activity when the composition is introduced into a plant growth medium (e.g., a soil).

In some embodiments, compositions of the present disclosure comprise Yersinia in an effective amount/concentration for enhancing plant growth/yield and/or for insecticidal or other pesticidal activity when the composition is applied to a plant or plant part.

Uses, methods and compositions of the present disclosure may comprise/utilize any suitable toxin (or other component) of/from Yersinia entomophaga and/or Yersinia nurmii, including, but not limited to, Yen-TC and components thereof (e.g., Chi1, Chi2, YenA1, YenA2, YenB, YenA1, YenC2).

Uses, methods and compositions of the present disclosure may comprise/utilize any suitable Bacillus thuringiensis, including, but not limited to, those expressly set forth herein.

Bacillus thuringiensis is a Gram-positive, spore-forming bacterium, that is possibly the most well-known microbial insecticide in existence. The organism was first isolated in 1901 and was first used as an insecticide in 1920 (http://www.bt.ucsd.edu/bt_history.html). The first commercial insecticide based on Bacillus thuringiensis was produced in France in 1938 (Ibrahim, M. A., et al., BIOENG. BUGS 1:31-50 (2010)). In the United States, Bacillus thuringiensis products were first manufactured commercially in 1958 and first registered for insecticidal use in 1961.

Bacillus thuringiensis is an insect pathogen that forms parasporal inclusions (i.e., crystals) during sporulation. The crystals contain proteins (S-endotoxins), including pore-forming Crystal (Cry) and cytolytic (Cyt) toxins (Bravo, A., et al., INSECT BIOCHEM. MOL. BIOL. 41:423-431 (2011)), that generally kill insect larva when ingested. The organism also produces insecticidal proteins that are not Cry or Cyt proteins. Vegetative insecticidal proteins (Vips) are one example. Use of the Bacillus thuringiensis insectical proteins that are not Cry or Cyt proteins, and variants thereof, in combination with Yersinia and/or Yersinia toxins, are encompassed by this disclosure. Toxins and other components may be found in and isolated from the supernatants of media in which Bacillus thuringiensis has been cultured. Thus, in some embodiments of the present disclosure, toxins and other Yersinia components are present in (and applied as part of) a cell-free Bacillus thuringiensis culture filtrate. In other embodiments, toxins and other Bacillus thuringiensis components are present (and applied as) isolate molecules that are substantially purified from the cells and culture media from which they are derived.

Different strains of Bacillus thuringiensis can have different insect host ranges. Generally, Bacillus thuringiensis strains have activity against lepidopteran, dipteran and/or coleopteran insects. Strains active against insects in the orders Hymenoptera, Homoptera, Orthoptera and/or Mallophaga have been identified. Some strains of Bacillus thuringiensis are active against nematodes, mites and/or protozoa.

A variety of products containing Bacillus thuringiensis have been produced. Generally, any of the Bacillus thuringiensis organisms from these products may be used in the compositions and methods disclosed herein. Some of these Bacillus thuringiensis products are set forth below: Agree®, containing Bacillus thuringiensis subspecies aizawai strain GC-91; Turex®, containing Bacillus thuringiensis subspecies aizawai strain GC-91; XenTari®, containing Bacillus thuringiensis subspecies aizawai; AQUABAC®, containing Bacillus thuringiensis subspecies israelensis strain BMP 144; VectoBac®, containing Bacillus thuringiensis subspecies israelensis strain AM65-52; Biobit®, containing Bacillus thuringiensis subspecies kurstaki; BIOLEP, containing Bacillus thuringiensis subspecies kurstaki strain Z-52; BMP 123, containing Bacillus thuringiensis subspecies kurstaki strain BMP 123; CoStar®, containing Bacillus thuringiensis subspecies kurstaki strain SA-12; Crymax®, containing Bacillus thuringiensis subspecies kurstaki strain EG 7841; Deliver®, containing Bacillus thuringiensis subspecies kurstaki strain SA-12; DiPel®, containing Bacillus thuringiensis subspecies kurstaki strain ABTS-351; Javelin®, containing Bacillus thuringiensis subspecies kurstaki strain SA-11; Lepinox®, containing Bacillus thuringiensis subspecies kurstaki strain EG 7826; LIPEL®, containing Bacillus thuringiensis subspecies kurstaki strain NCIM 2514; Thuricide®, containing Bacillus thuringiensis subspecies kurstaki strain SA-12; and Novodor®, containing Bacillus thuringiensis subspecies tenebrionislmorrisoni strain NB-176.

Some other strains or designations of Bacillus thuringiensis may include: Bacillus thuringiensis ATCC 13367, Bacillus thuringiensis NRRL B-21619, Bacillus thuringiensis ABTS-1857, Bacillus thuringiensis SAN 401 I, Bacillus thuringiensis ABG-6305, Bacillus thuringiensis ABG-6346, Bacillus thuringiensis SB4, Bacillus thuringiensis HD-1, Bacillus thuringiensis EG 2348, Bacillus thuringiensis EG 7841, Bacillus thuringiensis DSM 2803, Bacillus thuringiensis NB-125, and Bacillus thuringiensis NB-176.

Bacillus thuringiensis strains are readily available to the public. Strains are available from numerous biological depositories. Strains can be cultured from commercially available products. Patents claiming aspects of Bacillus thuringiensis strains also have issued and now expired, where the patent claims are supported by biological deposits. Those Bacillus thuringiensis strains are now available from the patent depositories.

The present disclosure provides cultures comprising, consisting essentially of or consisting of the Bacillus thuringiensis disclosed herein. In some embodiments, at least 95, 96, 97, 98, 99, 99.5, 99.6, 99.7, 99.8, or 99.9% of subcultures taken from the culture exhibit a genotype that is at least 95, 96, 97, 98, 99%, 99.1%, 99.2%, 99.3%, 99.4%, 99.5%, 99.55%, 99.6%, 99.65%, 99.7%, 99.75%, 99.8%, 99.85%, 99.9%, 99.95%, or 100% identical to that of the Bacillus thuringiensis disclosed herein. In some embodiments, the culture is a biologically pure culture of the Bacillus thuringiensis. These may be combined with similar cultures of Yersinia.

In some examples, crystal proteins, toxin proteins, and the like (Cry, Cyt, non-Cry, non-Cyt proteins), including vegetative insecticidal proteins (Vips), Bin-like proteins, ETX_MTX2-family proteins and insecticidal (Sip) toxins, from any of the above Bacillus thuringiensis strains, or at least 15, 20, 25, 30, 35, 40, 45, 50, 55, 60, 65, 70, 75, 80, 85, 90, 95, 96, 97, 98 or 99% identical to the proteins in those, or any other strains of Bacillus thuringiensis may be used in the compositions and methods disclosed herein. The crystal proteins, toxin proteins, and the like (Palma, L , et al., 2014. Bacillus thuringiensis Toxins: An Overview of Their Biocidal Activity. Toxins (Basel) 6, 3296-3325) may be purified or partially purified away from other, non-toxin components. Purification or partial purification of the proteins and/or subunits may use standard protein purification methodologies that are known in the art.

Bacillus thuringiensis may be incorporated into compositions in any suitable amount/concentration. The absolute value of the amount/concentration that is/are sufficient to cause the desired effect(s) may be affected by factors such as the type, size and volume of material to which the composition will be applied and storage conditions (e.g., temperature, relative humidity, duration). Those skilled in the art will understand how to select an effective amount/concentration using routine dose-response experiments.

In some embodiments, compositions of the present disclosure comprise Bacillus thuringiensis in an amount ranging from about 1×10¹ to about 1×10¹² colony-forming units (CFU) per gram and/or milliliter of composition. For example, compositions of the present disclosure may comprise about 1×10¹, 1×10², 1×10³, 1×10⁴, 1×10⁵, 1×10⁶, 1×10⁷, 1×10⁸, 1×10⁹, 1×10¹⁰, 1×10¹¹, 1×10¹² or more CFU of Bacillus thuringiensis per gram and/or milliliter of composition. In some embodiments, compositions of the present disclosure comprise at least 1×10⁴, 1×10⁵, 1×10⁶, 1×10⁷, 1×10⁸, 1×10⁹, 1×10¹⁰, 1×10¹¹, 1×10¹² CFU of Bacillus thuringiensis per gram and/or milliliter of composition.

In some embodiments, Bacillus thuringiensis comprises about 0.00000001 to about 95% (by weight) of the composition. In some examples, compositions of the present disclosure may comprise about 1×10⁻¹⁵, 1×10⁻¹⁴, 1×10⁻¹³, 1×10⁻¹², 1×10⁻¹¹, 1×10⁻¹⁰, 1×10⁻⁹, 1×10⁻⁸, 1×10⁻⁷, 1×10⁻⁶, 1×10⁻⁵, 1×10⁻⁴, 1×10⁻³, 1×10⁻², 1×10⁻¹, 0.2, 0.3, 0.4, 0.5, 0.6, 0.7, 0.8, 0.9, 1, 1.25, 1.5, 1.75, 2, 2.25, 2.5, 2.75, 3, 3.25, 3.5, 3.75, 4, 4.25, 4.5, 4.75, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 55, 60, 65, 70, 75, 80, 85, 90, 95% or more (by weight) of Bacillus thuringiensis. In some embodiments, Bacillus thuringiensis comprises about 1 to about 25%, about 5 to about 20%, about 5 to about 15%, about 5 to about 10% or about 8 to about 12% (by weight) of the composition.

In some embodiments, compositions of the present disclosure comprise Bacillus thuringiensis in an effective amount/concentration for enhancing plant growth/yield and/or for insecticidal or other pesticidal activity when the composition is introduced into a plant growth medium (e.g., a soil).

In some embodiments, compositions of the present disclosure comprise Bacillus thuringiensis in an effective amount/concentration for enhancing plant growth/yield and/or for insecticidal activity when the composition is applied to a plant or plant part.

Uses, methods and compositions of the present disclosure may comprise/utilize any suitable toxin (or other component) of/from Bacillus thuringie, including, but not limited to, δ-endotoxins, such as Cry toxins and Cyt toxins.

Combinations of Yersinia and Bacillus thuringiensis provide advantages as insecticides, as compared to Yersinia alone and Bacillus thuringiensis alone. In some examples, the insecticidal activity (e.g., the magnitude of insecticidal activity) of the combination is unexpected as compared to insecticidal activity of Yersinia entomophaga and Bacillus thuringiensis alone. In some examples, where some or all insects possess or have developed resistance to insecticidal effects of one or both bacteria, combinations of Yersinia and Bacillus thuringiensis may restore insecticidal activity and/or slow/prevent development of this resistance among one or more types of insects. In some examples, combinations of Yersinia and Bacillus thuringiensis may provide a different insect host range as compared to the combined insect host range of the Yersinia and the Bacillus thuringiensis alone. As will be understood by those skilled in the art, there may also be advantages in using combinations of Yersinia and Bacillus thuringiensis that produce additive, or even antagonistic, effects. In some examples, the amount of one of the agents may be reduced as compared to the amount of the agent used to produce the same effect alone.

Generally, the effects of the combinations, insecticidal or otherwise, may be determined using assays known in the art. Generally, these assays, including the assays used in the studies described in the Examples of this application, robustly reflect efficacy/activity/results of the compositions in the field. In some examples, the effects of the combinations of Yersinia entomophaga and Bacillus thuringiensis may be unexpected, additive or antagonistic on insect control and/or plant growth and/or yield (e.g., enhanced plant growth and/or enhanced plant yield). Although unexpected or even additive effects of the combinations are thought to be most advantageous, in some examples, there may be advantages, in particular antagonistic effects, of the combinations.

In addition to combinations of one or more Yersinia entomophaga (and/or one or more toxins or other components derived therefrom) and/or one or more Yersinia nurmii (and/or one or more toxins or other components derived therefrom) with one or more Bacillus thuringiensis (and/or one or more toxins or other components derived therefrom), compositions of the present disclosure may contain one or more additional components.

In some examples, the disclosed compositions may contain one or more pesticides and/or one or more other substances. Pesticidal agents may include chemical pesticides and biopesticides or biocontrol agents. Various types of chemical pesticides and biopesticides include acaricides, insecticides, nematicides, fungicides, gastropodicides, herbicides, virucides, bactericides, miticides and combinations thereof. Biopesticides or biocontrol agents may include bacteria, fungi, beneficial nematodes, and viruses that exhibit pesticidal activity. Compositions may comprise other agents for pest control, such as microbial extracts, plant growth activators, and/or plant defense agents. Biostimulants and/or plant signal molecules may be part of the compositions in some examples.

In some examples, one or more of the other pesticides or other substances may be specifically excluded from the disclosed compositions. In some examples, one or more of animal repellents, acaracides, antimicrobials, avicides, bactericides, disinfectants and/or sanitizers, gastropodicides, fungicides, herbicides, insecticides, insect growth regulators, insect repellents, miticides, molluscicides, nematicides, plant signal molecules, predacidse, piscicides, rodenticides, termiticides, viricides, and the like, may specifically be excluded from the disclosed compositions.

In some examples, the number of different Yersinia organisms or strains and/or Bacillus thuringiensis organisms or strains in the disclosed compositions is not limited. Likewise, the number of different pesticides and/or other substances within a disclosed combination is not limited. A composition may contain one Yersinia, one Bacillus, and one pesticide or other substance. A combination may contain multiple Yersinia strains, multiple Bacillus strains, and multiple pesticides, insecticides and/or other substances. Individual components of a combination may be combined as part of a manufacturing or formulation process or may be combined immediately prior to use. In some examples, individual components of a combination may not be combined until they are applied to a plant (e.g., individual components are applied separately to plants, but simultaneously or at about the same time).

In some examples, the Yersinia and Bacillus thuringiensis (organisms and/or toxins) are applied to plants at the same time. In some examples, the Yersinia is applied to the plant before the Bacillus thuringiensis is applied to the plant. In some examples, the Bacillus thuringiensis is applied to the plant before the Yersinia is applied to the plant. In some examples, one of the organisms or toxins (Yersinia or Bacillus thuringiensis) are applied minutes, hours, days, weeks, or months before the other of the organisms or toxins. In some examples, one of the organisms or toxins is about applied 5, 15 or 30 minutes, 1, 2, 3, 4, 5, 6, 7, 8, 9, 10, 11, 12, 18 or 24 hours, 1, 2, 3, 4, 5, 6, 7 days, 1, 2, 3, 4, 5, 6, 7 or 8 weeks, or 1, 2, 3, 4, 5, 6, 8, 10 or 12 months before the other of the organisms or toxins.

Generally, the individual components are present in at least an effective amount within the composition. In some examples, the compositions containing these individual components may produce unexpected effects as compared to the effects of individual components of the combination. These effects may be one or more of pesticidal, insecticidal, enhanced plant growth, enhanced plant yield, and the like. In some examples, the compositions containing these individual components produce additive effects as compared to the effects of individual components of the combination. The additive effects may be one or more of pesticidal, insecticidal, enhanced plant growth, enhanced plant yield, and the like. In some examples, the compositions containing these individual components may produce antagonistic effects as compared to the effects of individual components of the combination.

Below are described substances that may be combined with Yersinia and/or Bacillus to obtain the compositions that are the subject of this application. The disclosed pesticides/other substances generally are grouped (e.g., Group 1, fungicides; Group 2, gastropodicides; Group 3, herbicides; Group 4, insecticides and/or nematicides; Group 5, acaracides and/or miticides; Group 6, biostimulants; Group 7, plant signal molecules; Group 8, other microbes) for the purposes of this disclosure. Each group generally contains pesticides/other substances that generally produce similar activities, within the contexts applicable herein (e.g., individual fungicides generally are active against fungi). However, herein, individual substances placed into the same group may have different levels of an activity, may produce their activities under different conditions and/or circumstances, and may have more than one activity (and, therefore, appear in more than one group). The groupings herein, therefore, are generally qualitative rather than quantitative, and facilitate drafting the claims, which are directed to many different combinations.

### Fungicides (Group 1)

Herein, the substances described in this section are part of Group 1. Fungicides may be selected to provide effective control against a broad spectrum of phytopathogenic fungi (and fungus-like organisms), including, but not limited to, soil-borne fungi from the classes Ascomycetes, Basidiomycetes, Chytridiomycetes, Deuteromycetes (syn. Fungi imperfecti), Peronosporomycetes (syn. Oomycetes), Plasmodiophoromycetes and Zygomycetes. According to some embodiments, the compositions comprise a fungicide (or combination of fungicides) that is toxic to one or more strains of Albugo (e.g., A. candida), Alternaria (e.g. A. alternata), Aspergillus (e.g., A. candidus, A. clavatus, A. flavus, A. fumigatus, A. parasiticus, A. restrictus, A. sojae, A. solani), Blumeria (e.g., B. graminis), Botrytis (e.g., B. cinerea), Cladosporum (e.g., C. cladosporioides), Colletofrichum (e.g., C. acutatum, C. boninense, C. capsici, C. caudatum, C. coccodes, C. crassipes, C. dematium, C. destructivum, C. fragariae, C. gloeosporioides, C. graminicola, C. kehawee, C. lindemuthianum, C. musae, C. orbiculare, C. spinaceae, C. sublineolum, C. trifolii, C. truncatum), Fusarium (e.g., F. graminearum, F. moniliforme, F. oxysporum, F. roseum, F. tricinctum), Helminthosporium, Magnaporthe (e.g., M. grisea, M. oryzae), Melamspora (e.g., M. lini), Mycosphaerella (e.g., M. graminicola), Nematospora, Penicillium (e.g., P. rugulosum, P. verrucosum), Phakopsora (e.g., P. pachyrhizi), Phomopsis, Phytiphtoria (e.g., P. infestans), Puccinia (e.g., P. graminis, P. sfriiformis, P. tritici, P. triticina), Pucivinia (e.g., P. graministice), Pythium, Pytophthora, Rhizoctonia (e.g., R. solani), Scopulariopsis, Selerotinia, Thielaviopsis and/or Ustilago (e.g., U. maydis). Additional examples of fungi may be found in Bradley, Managing Diseases, in ILLINOIS AGRONOMY HANDBOOK (2008).

In some embodiments, compositions of the present disclosure comprise one or more chemical fungicides and Yersinia. Non-limiting examples of chemical fungicides include strobilurins, such as azoxystrobin, coumethoxystrobin, coumoxystrobin, dimoxystrobin, enestroburin, fluoxastrobin, kresoxim-methyl, metominostrobin, orysastrobin, picoxystrobin, pyraclostrobin, pyrametostrobin, pyraoxystrobin, pyribencarb, trifloxystrobin, 2-[2-(2,5-dimethyl-phenoxymethyl)-phenyl]-3-methoxy-acrylic acid methyl ester and 2-(2-(3-(2,6-dichlorophenyl)-1-methyl-allylideneaminooxymethyl)-phenyl)-2-methoxyimino-N-methyl-acetamide; carboxamides, such as carboxanilides (e.g., benalaxyl, benalaxyl-M, benodanil, bixafen, boscalid, carboxin, fenfuram, fenhexamid, flutolanil, fluxapyroxad, furametpyr, isopyrazam, isotianil, kiralaxyl, mepronil, metalaxyl, metalaxyl-M (mefenoxam), ofurace, oxadixyl, oxycarboxin, penflufen, penthiopyrad, sedaxane, tecloftalam, thifluzamide, tiadinil, 2-amino-4-methyl-thiazole-5-carboxanilide, N-(4′-trifluoromethylthiobiphenyl-2-yl)-3-difluoromethyl-1-methyl-1H-pyrazole-4-carboxamide, N-(2-(1,3,3-trimethylbutyl)-phenyl)-1,3-dimethyl-5-fluoro-1H-pyrazole-4-carboxamide), carboxylic morpholides (e.g., dimethomorph, flumorph, pyrimorph), benzoic acid amides (e.g., flumetover, fluopicolide, fluopyram, zoxamide), carpropamid, dicyclomet, mandiproamid, oxytetracyclin, silthiofam and N-(6-methoxy-pyridin-3-yl) cyclopropanecarboxylic acid amide; azoles, such as triazoles (e.g., azaconazole, bitertanol, bromuconazole, cyproconazole, difenoconazole, diniconazole, diniconazole-M, epoxiconazole, fenbuconazole, fluquinconazole, flusilazole, flutriafol, hexaconazole, imibenconazole, ipconazole, metconazole, myclobutanil, oxpoconazole, paclobutrazole, penconazole, propiconazole, prothioconazole, simeconazole, tebuconazole, tetraconazole, triadimefon, triadimenol, triticonazole, uniconazole) and imidazoles (e.g., cyazofamid, imazalil, pefurazoate, prochloraz, triflumizol); heterocyclic compounds, such as pyridines (e.g., fluazinam, pyrifenox (cf.D1b), 3-[5-(4-chloro-phenyl)-2,3-dimethyl-isoxazolidin-3-yl]-pyridine, 3-[5-(4-methyl-phenyl)-2,3-dimethyl-isoxazolidin-3-yl]-pyridine), pyrimidines (e.g., bupirimate, cyprodinil, diflumetorim, fenarimol, ferimzone, mepanipyrim, nitrapyrin, nuarimol, pyrimethanil), piperazines (e.g., triforine), pirroles (e.g., fenpiclonil, fludioxonil), morpholines (e.g., aldimorph, dodemorph, dodemorph-acetate, fenpropimorph, tridemorph), piperidines (e.g., fenpropidin), dicarboximides (e.g., fluoroimid, iprodione, procymidone, vinclozolin), non-aromatic 5-membered heterocycles (e.g., famoxadone, fenamidone, flutianil, octhilinone, probenazole, 5-amino-2-isopropyl-3-oxo-4-ortho-tolyl-2,3-dihydro-pyrazole-1-carbothioic acid S-allyl ester), acibenzolar-S-methyl, ametoctradin, amisulbrom, anilazin, blasticidin-S, captafol, captan, chinomethionat, dazomet, debacarb, diclomezine, difenzoquat, difenzoquat-methylsulfate, fenoxanil, Folpet, oxolinic acid, piperalin, proquinazid, pyroquilon, quinoxyfen, triazoxide, tricyclazole, 2-butoxy-6-iodo-3-propylchromen-4-one, 5-chloro-1-(4,6-dimethoxy-pyrimidin-2-yl)-2-methyl-1H-benzoimidazole and 5-chloro-7-(4-methylpiperidin-1-yl)-6-(2,4,6-trifluorophenyl)-[1,2,4]triazolo-[1,5-a]pyrimidine; benzimidazoles, such as carbendazim; and other active substances, such as guanidines (e.g., guanidine, dodine, dodine free base, guazatine, guazatine-acetate, iminoctadine), iminoctadine-triacetate and iminoctadine-tris(albesilate); antibiotics (e.g., kasugamycin, kasugamycin hydrochloride-hydrate, streptomycin, polyoxine and validamycin A); nitrophenyl derivates (e.g., binapacryl, dicloran, dinobuton, dinocap, nitrothal-isopropyl, tecnazen); organometal compounds (e.g., fentin salts, such as fentin-acetate, fentin chloride, fentin hydroxide); sulfur-containing heterocyclyl compounds (e.g., dithianon, isoprothiolane); organophosphorus compounds (e.g., edifenphos, fosetyl, fosetyl-aluminum, iprobenfos, phosphorus acid and its salts, pyrazophos, tolclofos-methyl); organochlorine compounds (e.g., chlorothalonil, dichlofluanid, dichlorophen, flusulfamide, hexachlorobenzene, pencycuron, pentachlorphenole and its salts, phthalide, quintozene, thiophanate-methyl, thiophanate, tolylfluanid, N-(4-chloro-2-nitro-phenyl)-N-ethyl-4-methyl-benzenesulfonamide) and inorganic active substances (e.g., Bordeaux mixture, copper acetate, copper hydroxide, copper oxychloride, basic copper sulfate, sulfur) and combinations thereof. In some embodiments, compositions of the present disclosure comprise acibenzolar-S-methyl, azoxystrobin, benalaxyl, bixafen, boscalid, carbendazim, cyproconazole, dimethomorph, epoxiconazole, fludioxonil, fluopyram, fluoxastrobin, flutianil, flutolanil, fluxapyroxad, fosetyl-A1, ipconazole, isopyrazam, kresoxim-methyl, mefenoxam, metalaxyl, metconazole, myclobutanil, orysastrobin, penflufen, penthiopyrad, picoxystrobin, propiconazole, prothioconazole, pyraclostrobin, sedaxane, silthiofam, tebuconazole, thiabendazole, thifluzamide, thiophanate, tolclofos-methyl, trifloxystrobin and triticonazole. In some embodiments, compositions of the present disclosure comprise azoxystrobin, pyraclostrobin, fluoxastrobin, trifloxystrobin, ipconazole, prothioconazole, sedaxane, fludioxonil, metalaxyl, mefenoxam, thiabendazole, fluxapyroxad and/or fluopyram. In some embodiments, compositions of the present disclosure comprise one or more aromatic hydrocarbons, benzimidazoles, benzthiadiazole, carboxamides, carboxylic acid amides, morpholines, phenylamides, phosphonates, quinone outside inhibitors (e.g. strobilurins), thiazolidines, thiophanates, thiophene carboxamides and/or triazoles.

In some examples, one or more of these fungicides may be specifically excluded from the compositions and methods disclosed herein.

### Gastropodicides (Group 2)

Herein, the substances described in this section are part of Group 2. There are a variety of substances that are known in the art to have activity against various gastropods. Some of these substances have activity against organisms other than gastropods. Some of these substances include methiocarb, metaldehyde, carbaryl, spinosad, copper sulfate in combination with lime, boric acid, diatomaceous earth, iron phosphate, and others.

In some examples, one or more of these gastropodicides may be specifically excluded from the compositions and methods disclosed herein.

### Herbicides (Group 3)

Herein, the substances described in this section are part of Group 3. Herbicides may be selected so as to provide effective control against a broad spectrum of plants, including, but not limited to, plants from the families Asteraceae, Caryophyllaceae, Poaceae and Polygonaceae. According to some embodiments, the composition comprises an herbicide (or combination of herbicides) that is toxic to one or more strains of Echinochloa (e.g., E. brevipedicellata, E. callopus, E. chacoensis, E. colona, E. crus-galli, E. crus-pavonis, E. elliptica, E. esculenta, E. frumentacea, E. glabrescens, E. haploclada, E. helodes, E. hokiformis, E. inundata, E. jaliscana, E. Jubata, E. kimberleyensis, E. lacunaria, E. macrandra, E. muricata, E. obtusiflora, E. oplismenoides, E. orzyoides, E. paludigena, E. picta, E. pithopus, E. polystachya, E. praestans, E. pyramidalis, E. rotundiflora, E. stagnina, E. telmatophila, E. turneriana, E. ugandensis, E. walteri), Fallopia (e.g., F. baldschuanica, F. japonica, F. sachalinensis), Stellaria (e.g., S. media) and/or Taraxacum (e.g., T. albidum, T. aphrogenes, T. brevicorniculatum, T. californicum, T. cenfrasiatum, T. ceratophorum, T. egthrospermum, T. farinosum, T. holmboei, T. japonicum, T. kok-saghyz, T. laevigatum T. officinale, T. platycarpum). Additional species of plants that may be targeted by compositions of the present disclosure may be found in Hager, Weed Management, in ILLINOIS AGRONOMY HANDBOOK (2008) and LOUX ET AL., WEED CONTROL GUIDE FOR OHIO, INDIANA AND ILLINOIS (2015).

In some embodiments, compositions of the present disclosure comprise one or more chemical herbicides. Non-limiting examples of chemical herbicides include 2,4-dichlorophenoxyacetic acid (2,4-D), 2,4,5-trichlorophenoxyacetic acid (2,4,5-T), ametryn, amicarbazone, aminocyclopyrachlor, acetochlor, acifluorfen, alachlor, atrazine, azafenidin, bentazon, benzofenap, bifenox, bromacil, bromoxynil, butachlor, butafenacil, butroxydim, carfentrazone-ethyl, chlorimuron, chlorotoluro, clethodim, clodinafop, clomazone, cyanazine, cycloxydim, cyhalofop, desmedipham, desmetryn, dicamba, diclofop, dimefuron, diuron, dithiopyr, fenoxaprop, fluazifop, fluazifop-P, fluometuron, flufenpyr-ethyl, flumiclorac-pentyl, flumioxazin, fluoroglycofen, fluthiacet-methyl, fomesafe, fomesafen, glyphosate, glufosinate, haloxyfop, hexazinone, imazamox, imazaquin, imazethapyr, ioxynil, isoproturon, isoxaflutole, lactofen, linuron, mecoprop, mecoprop-P, mesotrion, metamitron, metazochlor, methibenzuron , metolachlor (and S-metolachlor), metoxuron, metribuzin, monolinuron, oxadiargyl, oxadiazon, oxyfluorfen, phenmedipham, pretilachlor, profoxydim, prometon, prometry, propachlor, propanil, propaquizafop, propisochlor, pyraflufen-ethyl, pyrazon, pyrazolynate, pyrazoxyfen, pyridate, quizalofop, quizalofop-P (e.g., quizalofop-ethyl, quizalofop-P-ethyl, clodinafop-propargyl, cyhalofop-butyl, diclofop-methyl, fenoxaprop-P-ethyl, fluazifop-P-butyl, haloxyfop-methyl, haloxyfop-R-methyl), saflufenacil, sethoxydim, siduron, simazine, simetryn, sulcotrione, sulfentrazone, tebuthiuron, tembotrione, tepraloxydim, terbacil, terbumeton, terbuthylazine, thaxtomin (e.g., the thaxtomins described in U.S. Pat. No. 7,989,393), thenylchlor, tralkoxydim, triclopyr, trietazine, tropramezone, salts and esters thereof; racemic mixtures and resolved isomers thereof and combinations thereof. In some embodiments, compositions of the present disclosure comprise acetochlor, clethodim, dicamba, flumioxazin, fomesafen, glyphosate, glufosinate, mesotrione, quizalofop, saflufenacil, sulcotrione, S-3100 and/or 2,4-D. In some embodiments, compositions of the present disclosure comprise glyphosate, glufosinate, dicamba, 2,4-D, acetochlor, metolachlor, pyroxasulfone, flumioxazin, fomesafen, lactofen, metribuzin, mesotrione, and/or ethyl 2-((3-(2-chloro-4-fluoro-5-(3-methyl-2,6-dioxo-4-(trifluoromethyl)-2,3-dihydropyrimidin-1(6H)-yl)phenoxy)pyridin-2-yl)oxy)acetate. In some embodiments, compositions of the present disclosure comprise one or more acetyl CoA carboxylase (ACCase) inhibitors, acetolactate synthase (ALS) inhibitors, acetohydroxy acid synthase (AHAS) inhibitors, photosystem II inhibitors, photosystem I inhibitors, protoporphyrinogen oxidase (PPO or Protox) inhibitors, carotenoid biosynthesis inhibitors, enolpyruvyl shikimate-3-phosphate (EPSP) synthase inhibitor, glutamine synthetase inhibitor, dihydropteroate synthetase inhibitor, mitosis inhibitors, 4-hydroxyphenyl-pyruvate-dioxygenase (4-HPPD) inhibitors, synthetic auxins, auxin herbicide salts, auxin transport inhibitors, nucleic acid inhibitors and/or one or more salts, esters, racemic mixtures and/or resolved isomers thereof.

In some examples, one or more of these herbicides may be specifically excluded from the compositions and methods disclosed herein.

Insecticides and/or Nematicides (Group 4)

Herein, the substances described in this section are part of Group 4. Insecticides may be selected so as to provide effective control against a broad spectrum of insects, including, but not limited to, insects from the orders Coleoptera, Dermaptera, Diptera, Hemiptera, Homoptera, Hymenoptera, Lepidoptera, Orthoptera and Thysanoptera. For example, compositions of the present disclosure may comprise one or more insecticides toxic to insects from the families Acrididae, Aleytodidae, Anobiidae, Anthomyiidae, Aphididae, Bostrichidae, Bruchidae, Cecidomyiidae, Cerambycidae, Cercopidae, Chrysomelidae, Cicadellidae, Coccinellidae, Cryllotalpidae, Cucujidae, Curculionidae, Dermestidae, Elateridae, Gelechiidae, Lygaeidae, Meloidae, Membracidae, Miridae, Noctuidae, Pentatomidae, Pyralidae, Scarabaeidae, Silvanidae, Spingidae, Tenebrionidae and/or Thripidae. According to some embodiments, the composition comprises an insecticide (or combination of insecticides) that is toxic to one or more species of Acalymma, Acanthaoscelides (e.g., A. obtectus,), Anasa (e.g., A. tristis), Anasfrepha (e.g., A. ludens), Anoplophora (e.g., A. glabripennis), Anthonomus (e.g., A. eugenii), Acyrthosiphon (e.g., A. pisum), Bacfrocera (e.g. B. dosalis), Bemisia (e.g., B. argentifolii, B. tabaci), Brevicoryne (e.g., B. brassicae), Bruchidius (e.g., B. atrolineatus), Bruchus (e.g., B. atomarius, B. dentipes, B. lentis, B. pisorum and/or B. rufipes), Callosobruchus (e.g., C. chinensis, C. maculatus, C. rhodesianus, C. subinnotatus, C. theobromae), Caryedon (e.g., C. serratus), Cassadinae, Ceratitis (e.g., C. capitata), Chrysomelinae, Circulifer (e.g., C. tenellus), Criocerinae, Cryptocephalinae, Cryptolestes (e.g., C. ferrugineus, C. pusillis, C. pussilloides), Cylas (e.g., C. formicarius), Delia (e.g., D. antiqua), Diabrotica, Diaphania (e.g., D. nitidalis), Diaphorina (e.g., D. citri), Donaciinae, Ephestia (e.g, E. cautella, E. elutella, E., keuhniella), Epilachna (e.g., E. varivestris), Epiphyas (e.g., E. postvittana), Eumolpinae, Galerucinae, Helicoverpa (e.g., H. zea), Heteroligus (e.g., H. meles), Iobesia (e.g., I. botrana), Lamprosomatinae, Lasioderma (e.g., L. serricorne), Leptinotarsa (e.g., L. decemlineata), Leptoglossus, Liriomyza (e.g., L. trifolii), Manducca, Melittia (e.g., M. cucurbitae), Myzus (e.g., M. persicae), Nezara (e.g., N. viridula), Orzaephilus (e.g., O. merator, O. surinamensis), Ostrinia (e.g., O. nubilalis), Phthorimaea (e.g., P. operculella), Pieris (e.g., P. rapae), Plodia (e.g., P. interpunctella), Plutella (e.g., P. xylostella), Popillia (e.g., P. japonica), Prostephanus (e.g., P. truncates), Psila, Rhizopertha (e.g., R. dominica), Rhopalosiphum (e.g., R. maidis), Sagrinae, Solenopsis (e.g., S. Invicta), Spilopyrinae, Sitophilus (e.g., S. granaries, S. oryzae and/or S. zeamais), Sitotroga (e.g., S. cerealella), Spodoptera (e.g., S. frugiperda), Stegobium (e.g., S. paniceum), Synetinae, Tenebrio (e.g., T. malens and/or T. molitor), Thrips (e.g., T. tabaci), Trialeurodes (e.g., T. vaporariorum), Tribolium (e.g., T. castaneum and/or T. confusum), Trichoplusia (e.g., T. ni), Trogoderma (e.g., T. granarium) and Trogossitidae (e.g., T. mauritanicus). Additional species of insects that may be targeted by compositions of the present disclosure may be found in CAPINERA, HANDBOOK OF VEGETABLE PESTS (2001) and Steffey and Gray, Managing Insect Pests, in ILLINOIS AGRONOMY HANDBOOK (2008).

Nematicides may be selected so as to provide effective control against a broad spectrum of nematodes, including, but not limited to, phytoparasitic nematodes from the classes Chromadorea and Enoplea. According to some embodiments, the composition comprises a nematicide (or combination of nematicides) that is toxic to one or more strains of Anguina, Aphelenchoides, Belonolaimus, Bursaphelenchus, Ditylenchus, Globodera, Helicotylenchus, Heterodera, Hirschmanniella, Meloidogyne, Naccobus, Pratylenchus, Radopholus, Rotylenshulus, Trichodorus, Tylenchulus and/or Xiphinema. Additional species that may be targeted by compositions of the present disclosure may be found in CAPINERA, HANDBOOK OF VEGETABLE PESTS (2001) and Niblack, Nematodes, in ILLINOIS AGRONOMY HANDBOOK (2008).

In some embodiments, compositions of the present disclosure comprise one or more chemical insecticides and/or nematicides. Non-limiting examples of chemical insecticides and nematicides include acrinathrin, alpha-cypermethrin, betacyfluthrin, cyhalothrin, cypermethrin, deltamethrin, csfenvalcrate, etofenprox, fenpropathrin, fenvalerate, flucythrinate, fosthiazate, lambda-cyhalothrin, gamma-cyhalothrin, permethrin, tau-fluvalinate, transfluthrin, zeta-cypermethrin, cyfluthri, bifenthrin, tefluthrin, eflusilanat, fubfenprox, pyrethrin, resmethrin, imidacloprid, acetamiprid, thiamethoxam, nitenpyram, thiacloprid, dinotefuran, clothianidin, imidaclothiz, chlorfluazuron, diflubenzuron, lufenuron, teflubenzuron, triflumuron, novaluron, flufenoxuron, hexaflumuron, bistrifluoron, noviflumuron, buprofezin, cyromazine, methoxyfenozide, tebufenozide, halofenozide, chromafenozide, endosulfan, fipronil, ethiprole, pyrafluprole, pyriprole, flubendiamide, chlorantraniliprole (e.g., Rynaxypyr), cyazypyr, emamectin, emamectin benzoate, abamectin, ivermectin, milbemectin, lepimectin, tebufenpyrad, fenpyroximate, pyridaben, fenazaquin, pyrimidifen, tolfenpyrad, dicofol, cyenopyrafen, cyflumetofen, acequinocyl, fluacrypyrin, bifenazate, diafenthiuron, etoxazole, clofentezine, spinosad, triarathen, tetradifon, propargite, hexythiazox, bromopropylate, chinomethionat, amitraz, pyrifluquinazon, pymetrozine, flonicamid, pyriproxyfen, diofenolan, chlorfenapyr, metaflumizone, indoxacarb, chlorpyrifos, spirodiclofen, spiromesifen, spirotetramat, pyridalyl, spinetoram, acephate, triazophos, profenofos, oxamyl, spinetoram, fenamiphos, fenamipclothiahos, 4-{[(6-chloropyrid-3-yl)methyl](2,2-difluoroethyl)amino}furan-2(5H)-one, cadusaphos, carbaryl, carbofuran, ethoprophos, thiodicarb, aldicarb, aldoxycarb, metamidophos, methiocarb, sulfoxaflor, cyantraniliprole and tioxazofen and combinations thereof. In some embodiments, compositions of the present disclosure comprise abamectin, aldicarb, aldoxycarb, bifenthrin, carbofuran, chlorantraniliporle, chlothianidin, cyfluthrin, cyhalothrin, cypermethrin, cyantraniliprole, deltamethrin, dinotefuran, emamectin, ethiprole, fenamiphos, fipronil, flubendiamide, fosthiazate, imidacloprid, ivermectin, lambda-cyhalothrin, milbemectin, nitenpyram, oxamyl, permethrin, spinetoram, spinosad, spirodichlofen, spirotetramat, tefluthrin, thiacloprid, thiamethoxam and/or thiodicarb. In some embodiments, compositions of the present disclosure comprise one or more carbamates, diamides, macrocyclic lactones, neonicotinoids, organophosphates, phenylpyrazoles, pyrethrins, spinosyns, synthetic pyrethroids, tetronic acids and/or tetramic acids. In some embodiments, compositions of the present disclosure comprise an insecticide selected from the group consisting of clothianidin, thiamethoxam, imidacloprid, cyantraniliprole, chlorantraniliprole, fluopyram and tioxazafen.

In some examples, insecticides include methomyl, examples of which are Lannate® and Acinate 24 L, which has activity at least against armyworms; oxamyl, one example of which is Vydate®, which has activity at least against armyworms; carbaryl, one of which is Sevin®, which has activity at least against codling moths; acephate, one of which is Orthene®, which has activity at least against armyworms; Lambda-cyhalothrin, examples of which are Mustang Max®, Baythroid®, and Karate, which have activity at least against armyworms; esfenvalerate, one of which is Asara.®; fenpropathrin, one of which is Danitol®, which has activity at least against armyworms; spinosad, examples of which are Entrust®, PESTANAL® and Monterey Garden Insect Spray, which has activity at least against armyworms and/or codling moths; spinetoram, one of which is Radiant®, which has activity at least against codling moths; emamectin benzoate, one of which is Proclaim®; tebufenozide, one of which is Confirm®, which has activity at least against armyworms; methoxyfenozide, one of which is Intrepid®, which has activity at least against armyworms; ryaxypry, examples of which are Prevathon® and Coragen®; chlorantraniliprole, examples of which include Voliam® and Acelepryn™, which have activity at least against armyworms and codling moths; flubendiamide, examples of which include Fenos®, Toursismo®, Synapse™, Vetica™ and BELT®, which have activity at least against armyworms; indoxacarb, examples of which are Avaunt® and Steward®, which have activity at least against armyworms; CYD-X™, which has activity at least against codling moths; and novaluron, examples of which are Romon® and Pedestal®.

In some examples, one or more of these insecticides/nematicides may be specifically excluded from the compositions and methods disclosed herein.

Acaracides and/or Miticides (Group 5)

Herein, the substances described in this section are part of Group 5. There are a variety of substances that are known in the art to have activity against various acarides. Some of these substances have activity against organisms other than acarides. Non-limiting examples of acaracides/mitides may include carvacrol, sanguinarine, azobenzene, benzoximate, benzyl benzoate, bromopropylate, chlorbenside, chlorfenethol, chlorfenson, chlorfensulphide, chlorobenzilate, chloropropylate, cyflumetofen, DDT, dicofol, diphenyl sulfone, dofenapyn, fenson, fentrifanil, fluorbenside, genit, hexachlorophene, phenproxide, proclonol, tetradifon, tetrasul, benomyl, carbanolate, carbaryl, carbofuran, methiocarb, metolcarb, promacyl, propoxur, aldicarb, butocarboxim, oxamyl, thiocarboxime, thiofanox, bifenazate, binapacryl, dinex, dinobuton, dinocap-4, dinocap-6, dinocton, dinopenton, dinosulfon, dinoterbon, DNOC, amitraz, chlordimeform, chloromebuform, formetanate, formparanate, medimeform, semiamitraz, afoxolaner, fluralaner, sarolaner, tetranactin avermectin acaricides, abamectin, doramectin, eprinomectin, ivermectin, selamectin, milbemectin, milbemycin oxime, moxidectin, clofentezine, cyromazine, diflovidazin, dofenapyn, fluazuron, flubenzimine, flucycloxuron, flufenoxuron, hexythiazox, bromocyclen, camphechlor, DDT, dienochlor, endosulfan, lindane, chlorfenvinphos, crotoxyphos, dichlorvos, heptenophos, mevinphos, monocrotophos, naled, TEPP, tetrachlorvinphos, amidithion, amiton, azinphos-ethyl, azinphos-methyl, azothoate, benoxafos, bromophos, bromophos-ethyl, carbophenothion, chlorpyrifos, chlorthiophos, coumaphos, cyanthoate, demeton-O, demeton-S, demeton-O-methyl, demeton-S-methyl, demeton-S-methylsulphon, dialifos, diazinon, dimethoate, dioxathion, disulfoton, endothion, ethion, ethoate-methyl, formothion, malathion, mecarbam, methacrifos, omethoate, oxydeprofos, oxydisulfoton, parathion, phenkapton, phorate, phosalone, phosmet, phostin, phoxim, pirimiphos-methyl, prothidathion, prothoate, pyrimitate, quinalphos, quintiofos, sophamide, sulfotep, thiometon, triazophos, trifenofos, vamidothion, trichlorfon, isocarbophos, methamidophos, propetamphos, dimefox, mipafox, schradan, azocyclotin, cyhexatin, fenbutatin oxide, phostin, dichlofluanid, dialifos, phosmet, cyenopyrafen, fenpyroximate, pyflubumide, tebufenpyrad, acetoprole, fipronil, vaniliprole, acrinathrin, bifenthrin, brofluthrinate, cyhalothrin, alpha-cypermethrin, fenpropathrin, fenvalerate, flucythrinate, flumethrin, tau-fluvalinate, permethrin, halfenprox, pyrimidifen, chlorfenapyr, sanguinarine, chinomethionat, thioquinox, bifujunzhi, fluacrypyrim, flufenoxystrobin, pyriminostrobin, aramite, propargite, spirodiclofen, clofentezine, diflovidazin, flubenzimine, hexythiazox, fenothiocarb, chloromethiuron, diafenthiuron, acequinocyl, amidoflumet, arsenous oxide, clenpirin, closantel, crotamiton, cycloprate, cymiazole, disulfiram, etoxazole, fenazaflor, fenazaquin, fluenetil, mesulfen, MNAF, nifluridide, nikkomycins, pyridaben, sulfiram, sulfluramid, sulfur, thuringiensin, and triarathene.

In some examples, one or more of these acaracides/miticides may be specifically excluded from the compositions and methods disclosed herein.

### Biostimulants (Group 6)

Herein, the substances described in this section are part of Group 6. Compositions of the present disclosure may comprise any suitable biostimulant(s), including, but not limited to, seaweed extracts (e.g., Ascophyllum nodosum extracts, such as alginate, Ecklonia maxima extracts, etc.), myo-inositol, glycine and combinations thereof.

In some embodiments, compositions of the present disclosure comprise one or more biostimulants in an amount/concentration of about 0.0001 to about 5% or more (by weight) of the composition. In some embodiments, the biostimulant(s) (e.g., glycine and/or seaweed extract) comprise(s) about about 0.0001, 0.0002, 0.0003, 0.0004, 0.0005, 0.0006, 0.0007, 0.0008, 0.0009, 0.001, 0.0015, 0.002, 0.0025, 0.003, 0.0035, 0.004, 0.0045, 0.005, 0.0055, 0.006, 0.0065, 0.007, 0.0075, 0.008, 0.0085, 0.009, 0.0095, 0.01, 0.015, 0.02, 0.025, 0.03, 0.035, 0.04, 0.045, 0.05, 0.06, 0.07, 0.08, 0.09, 0.1, 0.02, 0.3, 0.4, 0.5, 0.6, 0.7, 0.8, 0.9, 1 to about 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4., 4.5, 4.6, 4.7, 4.8, 4.9, 5% (by weight) of the composition. For example, compositions of the present disclosure may comprise about 0.0005, 0.00075, 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.15, 0.2, 0.25, 0.3, 0.35, 0.4, 0.45, 0.5, 0.55, 0.6, 0.65, 0.7, 0.75, 0.8, 0.85, 0.9, 0.95, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4., 4.5, 4.6, 4.7, 4.8, 4.9, 5% or more (by weight) of one or more biostimulants (e.g., glycine and/or seaweed extract).

In some examples, one or more of these biostimulants may be specifically excluded from the compositions and methods disclosed herein.

### Plant Signal Molecules (Group 7)

Herein, the substances described in this section are part of Group 7. Compositions of the present disclosure may comprise any suitable plant signal molecule(s), including, but not limited to, lipo-chitooligosaccharides (LCDs), chitooligosaccharides (COs), chitinous compounds, flavonoids, non-flavonoid node-gene inducers, jasmonic acid or derivatives thereof, linoleic acid or derivatives thereof, linolenic acid or derivatives thereof and karrikins.

Compositions of the present disclosure may comprise any suitable LCO(s). LCOs, sometimes referred to as symbiotic nodulation (Nod) signals or Nod factors, consist of an oligosaccharide backbone of β-1,4-linked N-acetyl-D-glucosamine (“GIcNAc”) residues with an N-linked fatty acyl chain condensed at the non-reducing end. LCOs differ in the number of GIcNAc residues in the backbone, in the length and degree of saturation of the fatty acyl chain and in the substitutions of reducing and non-reducing sugar residues. See, e.g., Denarie, et al., ANN. REV. BIOCHEM. 65:503 (1996); Hamel, et al., PLANTA 232:787 (2010); Prome, et al., PURE & APPL. CHEM. 70(1):55 (1998).

Compositions of the present disclosure may comprise any suitable CO(s). COs, sometimes referred to as N-acetylchitooligosaccharides, are also composed of GIcNAc residues but have side chain decorations that make them different from chitin molecules [(C₈—H₁₃NO₅)ₙ, CAS No. 1398-61-4] and chitosan molecules [(C₅H₁₁NO₄)ₙ, CAS No. 9012-76-4]. See, e.g., D'Haeze et al., GLYCOBIOL. 12(6):79R (2002); Demont-Caulet et al., PLANT PHYSIOL. 120(1):83 (1999); Hanel et al., PLANTA 232:787 (2010); Muller et al., PLANT PHYSIOL.124:733 (2000); Robina et al., TETRAHEDRON 58:521-530 (2002); Rouge et al., Docking of Chitin Oligomers and Nod Factors on Lectin Domains of the LysM-RLK Receptors in the Medicago-Rhizobium Symbiosis, in THE MOLECULAR IMMUNOLOGY OF COMPLEX CARBOHYDRATES-3 (Springer Science, 2011); Van der Holst et al., CURR. OPIN. STRUC. BIOL. 11:608 (2001); Wan etal., PLANT CELL 21:1053 (2009); and PCT/F100/00803 (2000). COs differ from LCOs in that they lack the pendant fatty acid chain that is characteristic of LCOs.

Compositions of the present disclosure may comprise any suitable chitinous compound(s), including, but not limited to, chitin (IUPAC: N-[5-[[3-acetylamino-4,5-dihydroxy-6-(hydroxymethyl)oxan-2yl]methoxymethyl]-2-[[5-acetylamino-4,6-dihydroxy-2-(hydroxymethyl)oxan-3-yl]methoxymethyl]-4-hydroxy-6-(hydroxymethyl)oxan-3-ys]ethanamide), chitosan (IUPAC: 5-amino-6-[5-amino-6-[5-amino-4,6-dihydroxy-2(hydroxymethyl)oxan-3-yl]oxy-4-hydroxy-2-(hydroxymethyl)oxan-3-yl]oxy-2(hydroxymethyl)oxane-3,4-diol) and isomers, salts and solvates thereof.

Compositions of the present disclosure may comprise any suitable flavonoid(s), including, but not limited to, anthocyanidins, anthoxanthins, chalcones, coumarins, flavanones, flavanonols, flavans and isoflavonoids, as well as analogues, derivatives, hydrates, isomers, polymers, salts and solvates thereof.

Flavonoids are phenolic compounds having the general structure of two aromatic rings connected by a three-carbon bridge. Classes of flavonoids include are known in the art. See, e.g., Jain et al., J. PLANT BIOCHEM. & BIOTECHNOL. 11:1 (2002); Shaw et al., ENVIRON. MICROBIOL. 11:1867 (2006). Flavonoid compounds are commercially available, e.g., from Novozymes BioAg, Saskatoon, Canada; Natland International Corp., Research Triangle Park, N.C.; MP Biomedicals, Irvine, Calif.; LC Laboratories, Woburn Mass. Flavonoid compounds may be isolated from plants or seeds, e.g., as described in U.S. Pat. Nos. 5,702,752; 5,990,291; and 6,146,668. Flavonoid compounds may also be produced by genetically engineered organisms, such as yeast, as described in Ralston et al., PLANT PHYSIOL. 137:1375 (2005).

In some embodiments, compositions of the present disclosure comprise one or more anthocyanidins According to some embodiments, the composition comprises cyanidin, delphinidin, malvidin, pelargonidin, peonidin and/or petunidin.

In some embodiments, compositions of the present disclosure comprise one or more anthoxanthins According to some embodiments, the composition comprises one or more flavones (e.g., apigenin, baicalein, chrysin, 7,8-dihydroxyflavone, diosmin, flavoxate, 6-hydroxyflavone, luteolin, scutellarein, tangeritin and/or wogonin) and/or flavonols (e.g., amurensin, astragalin, azaleatin, azalein, fisetin, furanoflavonols galangin, gossypetin, 3-hydroxyflavone, hyperoside, icariin, isoquercetin, kaempferide, kaempferitrin, kaempferol, isorhamnetin, morin, myricetin, myricitrin, natsudaidain, pachypodol, pyranoflavonols quercetin, quericitin, rhamnazin, rhamnetin, robinin, rutin, spiraeoside, troxerutin and/or zanthorhamnin).

In some embodiments, compositions of the present disclosure comprise one or more flavanones. According to some embodiments, the composition comprises butin, eriodictyol, hesperetin, hesperidin, homoeriodictyol, isosakuranetin, naringenin, naringin, pinocembrin, poncirin, sakuranetin, sakuranin and/or sterubin.

In some embodiments, compositions of the present disclosure comprise one or more flavanonols. According to some embodiments, the composition comprises dihydrokaempferol and/or taxifolin.

In some embodiments, compositions of the present disclosure comprise one or more flavans. According to some embodiments, the composition comprises one or more flavan-3-ols (e.g., catechin (C), catechin 3-gallate (Cg), epicatechins (EC), epigallocatechin (EGC) epicatechin 3-gallate (ECg), epigallcatechin 3-gallate (EGCg), epiafzelechin, fisetinidol, gallocatechin (GC), gallcatechin 3-gallate (GCg), guibourtinidol, mesquitol, robinetinidol, theaflavin-3-gallate, theaflavin-3′-gallate, theflavin-3,3′-digallate, thearubigin), flavan-4-ols (e.g., apiforol and/or luteoforol) and/or flavan-3,4-diols (e.g., leucocyanidin, leucodelphinidin, leucofisetinidin, leucomalvidin, luecopelargonidin, leucopeonidin, leucorobinetinidin, melacacidin and/or teracacidin) and/or dimers, trimers, oligomers and/or polymers thereof (e.g., one or more proanthocyanidins).

In some embodiments, compositions of the present disclosure comprise one or more isoflavonoids. According to some embodiments, the composition comprises one or more isoflavones (e.g, biochanin A, daidzein, formononetin, genistein and/or glycitein), isoflavanes (e.g., equol, ionchocarpane and/or laxifloorane), isoflavandiols, isoflavenes (e.g., glabrene, haginin D and/or 2-methoxyjudaicin), coumestans (e.g., coumestrol, plicadin and/or wedelolactone), pterocarpans and/or roetonoids.

Compositions of the present disclosure may comprise any suitable flavonoid derivative, including, but not limited to, neoflavonoids (e.g, calophyllolide, coutareagenin, dalbergichromene, dalbergin, nivetin) and pterocarpans (e.g., bitucarpin A, bitucarpin B, erybraedin A, erybraedin B, erythrabyssin II, erthyrabissin-1, erycristagallin, glycinol, glyceollidins, glyceollins, glycyrrhizol, maackiain, medicarpin, morisianine, orientanol, phaseolin, pisatin, striatine, trifolirhizin).

Flavonoids and derivatives thereof may be incorporated into compositions of the present disclosure in any suitable form, including, but not limited to, polymorphic and crystalline forms.

Compositions of the present disclosure may comprise any suitable non-flavonoid nod-gene inducer(s), including, but not limited to, jasmonic acid ([1R-[1α,2β(Z)]]-3-oxo-2-(pentenyl)cyclopentaneacetic acid; JA), linoleic acid ((Z,Z)-9,12-Octadecadienoic acid) and linolenic acid ((Z,Z,Z)-9,12,15-octadecatrienoic acid), as well as analogues, derivatives, hydrates, isomers, polymers, salts and solvates thereof.

Jasmonic acid and its methyl ester, methyl jasmonate (MeJA), collectively known as jasmonates, are octadecanoid-based compounds that occur naturally in some plants (e.g., wheat), fungi (e.g., Botryodiplodia theobromae, Gibbrella fujikuroi), yeast (e.g., Saccharomyces cerevisiae) and bacteria (e.g., Escherichia coli). Linoleic acid and linolenic acid may be produced in the course of the biosynthesis of jasmonic acid. Jasmonates, linoleic acid and linolenic acid (and their derivatives) are reported to be inducers of nod gene expression or LCO production by rhizobacteria. See, e.g., Mabood, et al. PLANT PHYSIOL. BIOCHEM. 44(11):759 (2006); Mabood et al., AGR. J. 98(2):289 (2006); Mabood, et al., FIELD CROPS RES. 95(2-3):412 (2006); Mabood & Smith, Linoleic and linolenic acid induce the expression of nod genes in Bradyrhizobium japonicum USDA 3, PLANT BIOL. (2001). Non-limiting examples of derivatives of jasmonic acid, linoleic acid, linolenic acid include esters, amides, glycosides and salts. Representative esters are compounds in which the carboxyl group of linoleic acid, linolenic acid, or jasmonic acid has been replaced with a —COR group, where R is an —OR¹ group, in which R¹ is: an alkyl group, such as a C1-C8 unbranched or branched alkyl group, e.g., a methyl, ethyl or propyl group; an alkenyl group, such as a C2-C8 unbranched or branched alkenyl group; an alkynyl group, such as a C2-C8 unbranched or branched alkynyl group; an aryl group having, for example, 6 to 10 carbon atoms; or a heteroaryl group having, for example, 4 to 9 carbon atoms, wherein the heteroatoms in the heteroaryl group can be, for example, N, O, P, or S. Representative amides are compounds in which the carboxyl group of linoleic acid, linolenic acid, or jasmonic acid has been replaced with a —COR group, where R is an NR²R³ group, in which R² and R³ are independently: hydrogen; an alkyl group, such as a C1-C8 unbranched or branched alkyl group, e.g., a methyl, ethyl or propyl group; an alkenyl group, such as a C2-C8 unbranched or branched alkenyl group; an alkynyl group, such as a C2-C8 unbranched or branched alkynyl group; an aryl group having, for example, 6 to 10 carbon atoms; or a heteroaryl group having, for example, 4 to 9 carbon atoms, wherein the heteroatoms in the heteroaryl group can be, for example, N, O, P, or S. Esters may be prepared by known methods, such as acid-catalyzed nucleophilic addition, wherein the carboxylic acid is reacted with an alcohol in the presence of a catalytic amount of a mineral acid. Amides may also be prepared by known methods, such as by reacting the carboxylic acid with the appropriate amine in the presence of a coupling agent such as dicyclohexyl carbodiimide (DCC), under neutral conditions. Suitable salts of linoleic acid, linolenic acid and jasmonic acid include e.g., base addition salts. The bases that may be used as reagents to prepare metabolically acceptable base salts of these compounds include those derived from cations such as alkali metal cations (e.g., potassium and sodium) and alkaline earth metal cations (e.g., calcium and magnesium). These salts may be readily prepared by mixing together a solution of linoleic acid, linolenic acid, or jasmonic acid with a solution of the base. The salts may be precipitated from solution and be collected by filtration or may be recovered by other means such as by evaporation of the solvent.

Compositions of the present disclosure may comprise any suitable karrakin(s), including, but not limited to, 2H-furo[2,3-c]pyran-2-ones, as well as analogues, derivatives, hydrates, isomers, polymers, salts and solvates thereof.

In some examples, one or more of these plant signal molecules may be specifically excluded from the compositions and methods disclosed herein.

In some embodiments, Yersinia entomophaga (and/or Yersinia nurmii) and Bacillus thuringiensis are the only microbes in the compositions of the present disclosure.

In some embodiments, compositions of the present disclosure comprise one or more additional microorganisms. Any suitable microorganism(s) may be added, including, but not limited to, agriculturally beneficial microorganisms such as diazotrophs, phosphate-solubilizing microorganisms, mycorrhizal fungi and biopesticides. Selection of additional microbes (if any) will depend on the intended application(s).

Non-limiting examples of bacteria that may be included in compositions of the present disclosure include Azospirillum brasilense INTA Az-39, Bacillus amyloliquefaciens D747, Bacillus amyloliquefaciens NRRL B 50349, Bacillus amyloliquefaciens TJ1000, Bacillus amyloliquefaciens FZB24, Bacillus amyloliquefaciens FZB42, Bacillus amyloliquefaciens IN937a, Bacillus amyloliquefaciens IT-45, Bacillus amyloliquefaciens TJ1000, Bacillus amyloliquefaciens MBI600, Bacillus amyloliquefaciens BS27 (deposited as NRRL B-5015), Bacillus amyloliquefaciens BS2084 (deposited as NRRL B-50013), Bacillus amyloliquefaciens 15AP4 (deposited as ATCC PTA-6507), Bacillus amyloliquefaciens 3AP4 (deposited as ATCC PTA-6506), Bacillus amyloliquefaciens LSSA01 (deposited as NRRL B-50104), Bacillus amyloliquefaciens ABP278 (deposited as NRRL B-50634), Bacillus amyloliquefaciens 1013 (deposited as NRRL B-50509), Bacillus amyloliquefaciens 918 (deposited as NRRL B-50508), Bacillus amyloliquefaciens 22CP1 (deposited as ATCC PTA-6508) and Bacillus amyloliquefaciens BS18 (deposited as NRRL B-50633), Bacillus cereus I-1562, Bacillus firmus I-1582, Bacillus lichenformis BA842 (deposited as NRRL B-50516), Bacillus lichenformis BL21 (deposited as NRRL B-50134), Bacillus mycoides NRRL B-21664, Bacillus pumilus NRRL B 21662, Bacillus pumilus NRRL B-30087, Bacillus pumilus ATCC 55608, Bacillus pumilus ATCC 55609, Bacillus pumilus GB34, Bacillus pumilus KFP9F, Bacillus pumilus QST 2808, Bacillus subtilis ATCC 55078, Bacillus subtilis ATCC 55079, Bacillus subtilis MBI 600, Bacillus subtilis NRRL B-21661, Bacillus subtilis NRRL B-21665, Bacillus subtilis CX-9060, Bacillus subtilis GB03, Bacillus subtilis GB07, Bacillus subtilis QST-713, Bacillus subtilis FZB24, Bacillus subtilis D747, Bacillus subtilis 3BP5 (deposited as NRRL B-50510), any species of Bradyrhizobium or other rhizobia, Pseudomonas jessenii PS06, Rhizobium leguminosarum SO12A-2 (IDAC 080305-01), Sinorhizobium fredii CCBAU114, Sinorhizobium fredii USDA 205, Yersinia entomophaga O82KB8 and combinations thereof, as well as microorganisms having at least at least 75, 80, 85, 90, 95, 96, 97, 97.5. 98, 98.5, 99, 99.5, 99.6, 99.7, 99.8, 99.9% or more identical to any of the aforementioned strains on the basis of 16S rDNA sequence identity.

Non-limiting examples of fungi that may be included in compositions of the present disclosure include Gliocladium virens ATCC 52045, Gliocladium virens GL-21, Glomus intraradices RTI-801, Metarhizium anisopliae F52, PENI, Trichoderma asperellum SKT-1, Trichoderma asperellum ICC 012, Trichoderma atroviride LC52, Trichoderma afroviride CNCM 1-1237, Trichoderma fertile JM41R, Trichoderma gamsii ICC 080, Trichoderma hamatum ATCC 52198, Trichoderma harzianum ATCC 52445, Trichoderma harzianum KRL-AG2, Trichoderma harzianum T-22, Trichoderma harzianum TH-35, Trichoderma harzianum T-39, Trichoderma harzianum ICC012, Trichoderma reesi ATCC 28217, Trichoderma virens ATCC 58678, Trichoderma virens G1-3, Trichoderma virens GL-21, Trichoderma virens G-41, Trichoderma viridae ATCC 52440, Trichoderma viridae ICC080, Trichoderma viridae TV1 and combinations thereof, as well as microorganisms having at least at least 75, 80, 85, 90, 95, 96, 97, 97.5. 98, 98.5, 99, 99.5, 99.6, 99.7, 99.8, 99.9% or more identical to any of the aforementioned strains on the basis of internal transcribed spacer (ITS) and/or cytochrome c oxidase (CO1) sequence identity.

Non-limiting examples of mycorrhizal fungi that may be included in compositions of the present disclosure include mycorrhizal strains such as Gigaspora margarita, Glomus aggregatum, Glomus brasilianum, Glomus clarum, Glomus deserticola, Glomus etunicatum, Glomus intraradices, Glomus monosporum, Glomus mosseae, Laccaria bicolor, Laccaria laccata, Paraglomus brazilianum, Pisolithus tinctorius, Rhizopogon amylopogon, Rhizopogon fulvigleba, Rhizopogon luteolus, Rhizopogon villosuli, Scleroderma cepa and Scleroderma cifrinum and combinations thereof.

Additional microorganisms may be incorporated into compositions of the present disclosure in any suitable amount(s)/concentration(s). The absolute value of the amount/concentration that is/are sufficient to cause the desired effect(s) may be affected by factors such as the type, size and volume of material to which the composition will be applied, the microorganisms in the composition, the number of microorganisms in the composition, the stability of the microorganisms in the composition and storage conditions (e.g., temperature, relative humidity, duration). Those skilled in the art will understand how to select an effective amount/concentration using routine dose-response experiments. Guidance for the selection of appropriate amounts/concentrations can be found, for example, in International Patent Application Nos. PCT/US2016/050529 and PCT/US2016/050647 and U.S. Provisional Patent Application Nos. 62/296,798; 62/271,857; 62/347,773; 62/343,217; 62/296,784; 62/271,873; 62/347,785; 62/347,794; and 62/347,805.

In some embodiments, one or more additional microorganisms is/are present in an effective amount/concentration for fixing atmospheric nitrogen, solubilizing phosphate, controlling one or more phytopathogenic pests, enhancing stress tolerance and/or enhancing plant growth/yield when the composition is introduced into a plant growth medium (e.g., a soil).

In some embodiments, one or more additional microorganisms is/are present in an effective amount/concentration for fixing atmospheric nitrogen, solubilizing phosphate, controlling one or more phytopathogenic pests, enhancing stress tolerance and/or enhancing plant growth/yield when the composition is applied to a plant or plant part.

In some embodiments, one or more additional microorganisms is/are present in an amount ranging from about 1×10¹ to about 1×10¹² colony-forming units (CFU) per gram and/or millilitre of composition. According to some embodiments, the composition comprises about 1×10¹, 1×10², 1×10³, 1×10⁴, 1×10⁵, 1×10⁶, 1×10⁷, 1×10⁸, 1×10⁹, 1×10¹⁰, 1×10¹¹, 1×10¹² or more CFU of one or more additional microorganisms per gram and/or milliliter of composition (e.g., about 1×10⁴ to about 1×10⁹ CFU/g of Bacillus amyloliquefaciens TJ1000 (also known as 1BE, isolate ATCC BAA-390), Bradyrhizobium, Metarhizium anisopliae F52, PENI, Trichoderma vixens G1-3, and/or Yersinia entomophaga O82KB8). In some embodiments, compositions of the present disclosure comprise at least 1×10⁴, 1×10⁵, 1×10⁶, 1×10⁷, 1×10⁸, 1×10⁹, 1×10¹⁰, 1×10¹¹, 1×10¹² CFU of one or more additional microorganisms per gram and/or milliliter of composition.

In some embodiments, spores from one or more additional microorganims comprise about 0.1 to about 90% (by weight) of the composition. According to some embodiments, the composition comprises about 0.1, 0.2, 0.3, 0.4, 0.5, 0.6, 0.7, 0.8, 0.9, 1, 1.25, 1.5, 1.75, 2, 2.25, 2.5, 2.75, 3, 3.25, 3.5, 3.75, 4, 4.25, 4.5, 4.75, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 55, 60, 65, 70, 75, 80, 85, 90, 95% or more (by weight) of microbial spores from one or more additional microorganisms (e.g., about 10% Bacillus amyloliquefaciens TJ1000, Metarhizium anisopliae F52, Penicillium bilaiae ATCC 20851, Penicillium bilaiae RS7B-SD1 and/or Trichoderma virens G1-3 spores). In some embodiments, the amount/concentration of microbial spores from one or more additional microorganisms is about 1 to about 25%, about 5 to about 20%, about 5 to about 15%, about 5 to about 10% or about 8 to about 12% (by weight) of the composition.

It is to be understood that additional microorganisms in compositions of the present disclosure may comprise vegetative cells and/or dormant spores. According to some embodiments, at least 1, 5, 10, 15, 20, 25, 30, 35, 40, 45, 50, 55, 60, 65, 70, 75, 80, 85, 90, 95, 96, 97, 98, 99% or more additional microorganisms are present in compositions of the present disclosure as vegetative cells. According to some embodiments, at least 1, 5, 10, 15, 20, 25, 30, 35, 40, 45, 50, 55, 60, 65, 70, 75, 80, 85, 90, 95, 96, 97, 98, 99% or more additional microorganisms are present in compositions of the present disclosure as spores.

Compositions of the present disclosure may comprise any suitable microbial extract(s), including, but not limited to, bacterial extracts, fungal extracts and combinations thereof. In some embodiments, compositions of the present disclosure comprise one or more extracts of media comprising one or more diazotrophs, phosphate-solubilizing microorganisms and/or biopesticides. In some embodiments, compositions of the present disclosure comprise an extract of media comprising one or more of the microbial strains.

In some examples, one or more of these other microbes may be specifically excluded from the compositions and methods disclosed herein.

Generally, the compositions and methods disclosed herein may be active against any type of insect, including insects that are members of the orders Coleoptera, Diptera, Hymenoptera, Lepidoptera, Orthoptera and Thysanoptera. In some examples, Yersinia entomophaga and Bacillus thuringiensis may not be effective against insects in one or more of these orders.

In some examples, combinations of Yersinia entomophaga and Bacillus thuringiensis may be effective against insects that include Aedes mosquitos, cotton leafhoppers, Anopheline mosquitos, melon and cotton aphids, tobacco whiteflys, rice stem borers, bed bugs, cockroaches, house mosquitos, codling moths, Asian citrus psyllids, sugarcane borers, green-belly stink bugs, stink bugs, western flower thrips, tsetse flies, cotton bollworms, corn earworms, tobacco budworms, Colorado potato beetles, eggplant fruit borers, American serpentine leafminers, European grapevine moths, African cowpea thrips, pollen beatles, houseflies, green peach aphids, currant-lettuce aphids, brown planthoppers, European corn borers, European red mites, diamondback moths, cabbage stem flea beetles, birdcheny-oat aphids, sandflies, avocado thrips, blackflies, English grain aphids, white-backed planthoppers, beet armyworms, fall armyworms, cotton leafworms, twospotted spider mites, onion thrips, glasshouse whiteflies, kissing bugs, red flour beetles, tomato leafminers, cabbage loopers, black cutworms, grasshoppers, locusts, ants, and others. This list is not meant to be inclusive of the insects that may be affected by the compositions and methods disclosed herein.

In some examples, Yersinia entomophaga and Bacillus thuringiensis may not be effective against one or more insects that include Aedes mosquitos, cotton leafhoppers, Anopheline mosquitos, melon and cotton aphids, tobacco whiteflys, rice stem borers, bed bugs, cockroaches, house mosquitos, codling moths, Asian citrus psyllids, sugarcane borers, green-belly stink bugs, stink bugs, western flower thrips, tsetse flies, cotton bollworms, corn earworms, tobacco budworms, Colorado potato beetles, eggplant fruit borers, American serpentine leafminers, European grapevine moths, African cowpea thrips, pollen beatles, houseflies, green peach aphids, currant-lettuce aphids, brown planthoppers, European corn borers, European red mites, diamondback moths, cabbage stem flea beetles, birdcherry-oat aphids, sandflies, avocado thrips, blackflies, English grain aphids, white-backed planthoppers, beet armyworms, fall armyworms, cotton leafworms, twospotted spider mites, onion thrips, glasshouse whiteflies, kissing bugs, red flour beetles, tomato leafminers, cabbage loopers, black cutworms, grasshoppers, locusts, ants, and others. Therefore, one or more of these insects may be excluded from the claimed compositions and/or methods.

In some examples, Yersinia entomophaga and Bacillus thuringiensis may be effective against insects that include chewing pests, examples of which include: 1) Lepidoptera—codling moths, sugarcane borers, cotton bollworms, corn earworms, tobacco budworms, eggplant fruit borers, European grapevine moths, European corn borers, diamondback moths, beet armyworms, fall armyworms, cotton leafworms, cabbage loopers, black cutworms, Agrotis spp., black cutworm, cutworm, Helicoverpa spp., tomato fruitworm, Heliothis spp., swift moth, strawberry root worm, Egyptian cotton leafworm, armyworms, velvetbean caterpillar, southwestern corn borer, soybean looper, southern armyworm and green cloverworms; 2) Coleoptera—rice stem borers, Colorado potato beetles, pollen beetles, cabbage stem flea beetles, red flour beetles, emerald ash borer, wireworm, Asian longhorn beetle, black turfgrass ataenius, bean leaf beetle, plum curculio, pecan weevil, banded cucumber beetle, western spotted cucumber beetle, corn rootworm, oriental beetle, annual bluegrass weevil, melonontha, black vine weevil, garden chafer, white grub, flea beetle, Japanese beetle grub, viburnum leaf beetle, cryptomeria bark beetle, weevils, pea weevil, scarab grubs, bluegrass billbug, strophasoma weevil, confused flour beetle, ambrosia beetle and blister beetle; 3) Orthoptera—grasshoppers, locusts and crickets; 4) Diptera—Aedes mosquitos, Anopheline mosquitos, house mosquitos, tsetse flies, American serpentine leafminers, houseflies, sandflies, blackflies, tomato leafminers, mosquitos, fungus gnat, root maggots, onion maggot, cabbage root maggot, shore fly, cranefly and leatherjacket; 5) Hymenoptera—ants and wasps; 6) Blattodea—German cockroach, oriental cockroach, American cockroach and termites.

In some examples, Yersinia entomophaga and Bacillus thuringiensis may be effective against insects that include rasping pests, examples of which include: 1) Thysanoptera—western flower thrips, African cowpea thrips, avocado thrips, onion thrips, privet thrips, eastern flower thrips and chili thrips.

In some examples, Yersinia entomophaga and Bacillus thuringiensis may be effective against insects that include sucking pests, examples of which include: 1) Hemiptera—cotton leafhoppers, melon and cotton aphids, tobacco whiteflys, bed bugs, asian citrus psyllids, green-belly stink bugs, stink bugs, green peach aphids, currant-lettuce aphids, brown planthoppers, birdcherry-oat aphids, English grain aphids, white-backed planthoppers, glasshouse whiteflies, kissing bugs, root aphids, grape phylloxera, brown marmorated stink bug, lygus, bagrada bug, three cornered alfalfa hopper, chinch bugs, potato psyllid, cabbage aphid, green leafhopper and potato leafhopper; 2) Acari: European red mites, twospotted spider mites, rust mite, livestock ticks, blacklegged tick, pacific spider mite, varroa mite, dog tick and lonestar tick.

In some examples, Yersinia entomophaga and Bacillus thuringiensis may be effective against insects that include root aphids, grape phylloxera, brown marmorated stink bugs, lygus, bagrada bugs, three cornered alfalfa hoppers, green cloverworms, chinch bugs and blister beetles.

In some examples, any of the above-listed insects may be specifically excluded from the range of insects that the compositions and methods disclosed herein have activity.

Yersinia entomophaga and Bacillus thuringiensis may be formulated into any suitable type of composition, including, but not limited to, compositions for foliar applications, seed coatings and soil applications. In some examples, the Yersinia entomophaga and Bacillus thuringiensis may be formulated separately, and then combined before application, or the formulated compositions may be separately applied, to a plant for example. Separate compositions for prolonging viability of the organisms, for example, may take into account the differences between the organisms (e.g., Yersinia is Gram-negative, Bacillus is Gram-positive) and/or different forms of the two organisms that may be used (e.g., Yersinia vegetative cells; optional spores for Bacillus). In some examples, it may be possible to use a single formulated composition that contains both Yersinia entomophaga and Bacillus thuringiensis.

Compositions of the present disclosure may comprise any suitable carrier(s), including, but not limited to, foliar-compatible carriers, seed-compatible carriers and soil-compatible carriers. Selection of appropriate carrier materials will depend on the intended application(s) and the microorganism(s) present in the composition. In some embodiments, the carrier material(s) will be selected to provide a composition in the form of a liquid, gel, slurry, or solid. In some embodiments, the carrier will consist essentially of or consist of one or more stabilizing compounds.

In some embodiments, the composition comprises one or more solid carriers. According to some embodiments, the composition comprises one or more powders (e.g., wettable powders) and/or granules. Non-limiting examples of solid carriers include clays (e.g., attapulgite clays, montmorillonite clay, etc.), peat-based powders and granules, freeze-dried powders, spray-dried powders, spray-freeze-dried powders and combinations thereof.

In some embodiments, the formulated composition comprises one or more liquid and/or gel carriers. According to some embodiments, the composition comprises one or more non-aqueous solvents. According to some embodiments, the composition comprises one or more aqueous solvents (e.g., water). According to some embodiments, an aqueous solvent, such as water, may be combined with a co-solvent, such as ethyl lactate, methyl soyate/ethyl lactate co-solvent blends (e.g., STEPOSOL™, Stepan), isopropanol, acetone, 1,2-propanediol, n-alkylpyrrolidones (e.g., AGSOLEX™ wetting agents; Ashland, Inc., Covington, Ky.), petroleum based-oils (e.g., AROMATIC™ and SOLVESSO™ fluids; ExxonMobil Chemical Company, Spring, Tex.), isoparrafinic hyydrocarbons (e.g., ISOPAR™ fluids; ExxonMobil Chemical Company, Spring, Tex.), cycloparaffinic hydrocarbons (e.g., NAPPAR™ 6; ExxonMobil Chemical Company, Spring, Tex.), mineral spirits (e.g., VARSOL™; ExxonMobil Chemical Company, Spring, Tex.), and mineral oils (e.g., paraffin oil). According to some embodiments, the composition comprises one or more inorganic solvents, such as decane, dodecane, hexylether and nonan. According to some embodiments, the composition comprises one or more organic solvents, such as acetone, dichloromethane, ethanol, hexane, methanol, propan-2-ol and trichloroethylene. Non-limiting examples of liquid/gel carriers include oils (e.g., mineral oil, olive oil, peanut oil, soybean oil, sunflower oil), polyethylene glycols (e.g., PEG 200, PEG 300, PEG 400, etc.), propylene glycols (e.g., PPG-9, PPG-10, PPG-17, PPG-20, PPG-26, etc.), ethoxylated alcohols (e.g., TOMADOL® (Air Products and Chemicals, Inc., Allentown, Pa.), TERGITOL™ 15-S surfactants such as TERGITOL™ 15-S-9 (The Dow Chemical Company, Midland, Mich.), etc.), isoparrafinic hyydrocarbons (e.g., ISOPAR™, ISOPAR™ L, ISOPAR™ M, ISOPAR™ V; ExxonMobil Chemical Company, Spring, Tex.), pentadecane, polysorbates (e.g. polysorbate 20, polysorbate 40, polysorbate 60, polysorbate 80, etc.), silicones (siloxanes, trisiloxanes, etc.) and combinations thereof.

Additional examples of carriers may be found in BURGES, composition OF MICROBIAL BIOPESTICIDES: BENEFICIAL MICROORGANISMS, NEMATODES AND SEED TREATMENTS (Springer Science & Business Media) (2012); Inoue & Horikoshi, J. FERMENTATION BIOENG.71(3):194 (1991).

Compositions of the present disclosure may comprise any suitable stabilizing compound(s), including, but not limited to, maltodextrins, monosaccharides, disaccharides, oligosaccharides, sugar alcohols, humic acids, fulvic acids, malt extracts, peat extracts, betaines, prolines, sarcosines, peptones, skim milks, oxidation control components, hygroscopic polymers and UV protectants.

In some embodiments, the composition comprises one or more maltodextrins (e.g., one or more maltodextrins having a dextrose equivalent value (DEV) of about 3, 4, 5, 6, 7, 8, 9, 10, 11, 12, 13, 14, 15, 16, 17, 18, 19, 20, 21, 22, 23, 24, or 25). According to some embodiments, the composition comprises one or more maltodextrins having a DEV of about 5 to about 6, 7, 8, 9, 10, 11, 12, 14, 15, 16, 17, 18, 19 or 20, about 10 to about 11, 12, 14, 15, 16, 17, 18, 19 or 20, or about 15 to about 16, 17, 18, 19 or 20. According to some embodiments, the composition comprises a combination of maltodextrins having a DEV of about 5 to about 6, 7, 8, 9, 10, 11, 12, 14, 15, 16, 17, 18, 19 or 20, about 10 to about 11, 12, 14, 15, 16, 17, 18, 19 or 20, or about 15 to about 16, 17, 18, 19 or 20. Non-limiting examples of maltodextrins include MALTRIN® M040 (DEV=5; molecular weight=3600; Grain Processing Corporation, Muscatine, Iowa), MALTRIN® M100 (DEV=10; molecular weight=1800; Grain Processing Corporation, Muscatine, Iowa), MALTRIN® M150 (DEV=15; molecular weight=1200; Grain Processing Corporation, Muscatine, Iowa), MALTRIN® M180 (DEV=18; molecular weight=1050; Grain Processing Corporation, Muscatine, Iowa), MALTRIN® M200 (DEV=20; molecular weight=900; Grain Processing Corporation, Muscatine, Iowa), MALTRIN® M250 (DEV=25; molecular weight=720; Grain Processing Corporation, Muscatine, Iowa); MALTRIN QD® M580 (DEV=16.5-19.9; Grain Processing Corporation, Muscatine, Iowa); MALTRIN QD® M585 (DEV=15.0-19.9; Grain Processing Corporation, Muscatine, Iowa); MALTRIN QD® M600 (DEV=20.0-23.0; Grain Processing Corporation, Muscatine, Iowa); GLOBE® Plus 15 DE (Ingredion Inc., Westchester, Ill.); and combinations thereof.

In some embodiments, the composition comprises one or more monosaccharides (e.g., allose, altrose, arabinose, fructose, galactose, glucose, gulose, iodose, lyxose, mannose, ribose, talose, threose and/or xylose). According to some embodiments, the composition comprises glucose. According to some embodiments, the composition does not comprise glucose.

In some embodiments, the composition comprises one or more disaccharides (e.g., cellobiose, chitobiose, gentiobiose, gentiobiulose, isomaltose, kojibiose, lactose, lactulose, laminaribiose, maltose (e.g., maltose monohydrate, anhydrous maltose), maltulose, mannobiose, melibiose, melibiulose, nigerose, palatinose, rutinose, rutinulose, sophorose, sucrose, trehalose, turanose and/or xylobiose). According to some embodiments, the composition comprises maltose. According to some embodiments, the composition does not comprise maltose. According to some embodiments, the composition comprises trehalose. According to some embodiments, the composition does not comprise trehalose.

In some embodiments, the composition comprises one or more oligosaccharides (e.g., fructo-oligosaccharides, galacto-oligosaccharides, mannon-oligosaccharides and/or raffinose).

In some embodiments, the composition comprises one or more sugar alcohols (e.g., arabitol, erythritol, fucitol, galactitol, glycerol, iditol, inositol, isomalt, lactitol, maltitol, maltotetraitol, maltotriitol, mannitol, polyglycitol, ribitol, sorbitol, threitol, volemitol and/or xylitol).

In some embodiments, the composition comprises one or more humic acids (e.g., one or more leonardite humic acids, lignite humic acids, peat humic acids and water-extracted humic acids). In some embodiments, the composition comprises ammonium humate, boron humate, potassium humate and/or sodium humate. In some embodiments, one or more of ammonium humate, boron humate, potassium humate and sodium humate is/are excluded from the composition. Nonlimiting examples of humic acids that may be useful in embodiments of the present disclosure include MDL Number MFCD00147177 (CAS Number 1415-93-6), MDL Number MFCD00135560 (CAS Number 68131-04-4), MDL Number MFCS22495372 (CAS Number 68514-28-3), CAS Number 93924-35-7, and CAS Number 308067-45-0.

In some embodiments, the composition comprises one or more fulvic acids (e.g., one or more leonardite fulvic acids, lignite fulvic acids, peat fulvic acids and/or water-extracted fulvic acids). In some embodiments, the composition comprises ammonium fulvate, boron fulvate, potassium fulvate and/or sodium fulvate. In some embodiments, one or more of ammonium fulvate, boron fulvate, potassium fulvate and sodium fulvate is/are excluded from compositions of the present disclosure. Nonlimiting examples of fulvic acids that may be useful in embodiments of the present disclosure include MDL Number MFCD09838488 (CAS Number 479-66-3).

In some embodiments, the composition comprises one or more betaines (e.g., trimethylglycine).

In some embodiments, the composition comprises one or more peptones (e.g., bacterial peptones, meat peptones, milk peptones, vegetable peptones and yeast peptones).

In some embodiments, the composition comprises one or more oxidation control components (e.g., one or more antioxidants and/or oxygen scavengers). According to some embodiments, the composition comprises one or more oxygen scavengers, such as ascrobic acid, ascorbate salts, catechol and/or sodium hydrogen carbonate. According to some embodiments, the composition comprises one or more antioxidants, such as ascorbic acid, ascorbyl palmitate, ascorbyl stearate, calcium ascorbate, carotenoids, lipoic acid, phenolic compounds (e.g., flavonoids, flavones, flavonols), potassium ascorbate, sodium ascorbate, thiols (e.g., glutathione, lipoic acid, N-acetyl cysteine), tocopherols, tocotrienols, ubiquinone and/or uric acid. Non-limiting examples of antioxidants include those that are soluble in the cell membrane (e.g., alpha tocopherol (vitamin E), ascorbyl palmitate) and those that are soluble in water (e.g., ascorbic acid and isomers or ascorbic acid, sodium or potassium salts of ascorbic acid or isomers or ascorbic acid, glutathione, sodium or potassium salts of glutathione). In some embodiments, use of a membrane-soluble antioxidant necessitates the addition of one or more surfactants to adequately disperse the antioxidant within the composition. According to some embodiments, the composition is/comprises ascorbic acid and/or glutathione.

In some embodiments, the composition comprises one or more hygroscopic polymers (e.g., hygroscopic agars, albumins, alginates, carrageenans, celluloses, gums (e.g., cellulose gum, guar gum, gum arabic, gum combretum, xantham gum), methyl celluloses, nylons, pectins, polyacrylic acids, polycaprolactones, polycarbonates, polyethylene glycols (PEG), polyethylenimines (PEI), polylactides, polymethylacrylates (PMA), polyurethanes, polyvinyl alcohols (PVA), polyvinylpyrrolidones (PVP), propylene glycols, sodium carboxymethyl celluloses and/or starches). Non-limiting examples of polymers include AGRIMER™ polymers (e.g., 30, AL-10 LC, AL-22, AT/ATF, VA 3E, VA 31, VA 5E, VA 51, VA 6, VA 6E, VA 7E, VA 71, VEMA AN-216, VEMA AN-990, VEMA AN-1200, VEMA AN-1980, VEMA H-815MS; Ashland Specialty Ingredients, Wilmington, Del.), EASYSPERSE™ polymers (Ashland Specialty Ingredients, Wilmington, Del.); DISCO™ AG polymers (e.g., L-250, L-280, L-285, L-286, L-320, L-323, L-517, L-519, L-520, L800; Incotec Inc , Salinas, Calif.), KELZAN® polymers (Bri-Chem Supply Ltd., Calgary, Alberta, CA), SEEDWORX™ polymers (e.g., Bio 200; Aginnovation, LLC, Walnut Groove, Calif.), TICAXAN® xanthan powders, such as PRE-HYDRATED® TICAXAN® Rapid-3 Powder (TIC Gums, White Marsh, Md.) and combinations thereof. Additional examples of polymers may be found in Pouci, et al. Am. J. Agric. Biol. Sci. 3(1):299 (2008).

In some embodiments, the composition comprises one or more UV protectants (e.g., one or more aromatic amino acids (e.g., tryptophan, tyrosine), carotenoids, cinnamates, lignosulfonates (e.g., calcium lignosulfonate, sodium lignosulfonate), melanins, mycosporines, polyphenols and/or salicylates). Non-limiting examples of UV protectants include Borregaard LignoTech™ lignosulfonates (e.g., Borresperse 3A, Borresperse CA, Borresperse NA, Marasperse AG, Norlig A, Norlig 11D, Ufoxane 3A, Ultrazine NA, Vanisperse CB; Borregaard Lignotech, Sarpsborg, Norway) and combinations thereof. Additional examples of UV protectants may be found in Burges, composition of Microbial Biopesticides: Beneficial Microorganisms, Nematodes and Seed Treatments (Springer Science & Business Media) (2012).

Compositions of the present disclosure may comprise any suitable nutrient(s), including, but not limited to, organic acids (e.g., acetic acid, citric acid, lactic acid, malic acid, taurine, etc.), macrominerals (e.g., phosphorous, calcium, magnesium, potassium, sodium, iron, etc.), trace minerals (e.g., boron, cobalt, chloride, chromium, copper, fluoride, iodine, iron, manganese, molybdenum, selenium, zinc, etc.), vitamins, (e.g., vitamin A, vitamin B complex (i.e., vitamin B₁, vitamin B₂, vitamin B₃, vitamin B₅, vitamin B₆, vitamin B₇, vitamin B₈, vitamin B₉, vitamin B₁₂, choline) vitamin C, vitamin D, vitamin E, vitamin K, carotenoids (α-carotene, β-carotene, cryptoxanthin, lutein, lycopene, zeaxanthin, etc.) and combinations thereof. In some embodiments, composition of the present disclosure comprise phosphorous, boron, chlorine, copper, iron, manganese, molybdenum and/or zinc.

Compositions of the present disclosure may comprise any suitable pest attractant(s) and/or feeding stimulant(s), including, but not limited to, brevicomin, ceralure, codlelure, cue-lure, disparlure, dominicalure, eugenol, frontalin, gossyplure, grandlure, hexalure, ipsdienol, ipsenol, japonilure, latitlure, lineatin, litlure, looplure, medlure, megatomic acid, methyl eugenol, moguchun, α-multistriatin, muscalure, orfalure, oryctalure, ostramone, rescalure, siglure, sulcatol, trimedlure and/or trunc-call.

Compositions of the present disclosure may comprise gluconolactone and/or one or more analogues, derivatives, hydrates, isomers, polymers, salts and/or solvates thereof.

Compositions of the present disclosure may comprise any suitable excipient(s), including, but not limited to, dispersants, drying agents, anti-freezing agents, seed flowability agents, safeners, anti-settlign agents, pH buffers and adhesives.

Compositions of the present disclosure may comprise any suitable agriculturally acceptable dispersant(s), including, but not limited to, surfactants and wetting agents. Selection of appropriate dispersants will depend on the intended application(s) and the microorganism(s) present in the composition. In general, the dispersant(s) will have low toxicity for the microorganism(s) in the composition and for the plant part(s) to which the composition is to be applied. In some embodiments, the dispersant(s) will be selected to wet and/or emulsify one or more soils. Non-limiting examples of dispersants include Atlox™ (e.g., 4916, 4991; Croda International PLC, Edison, N.J.), Atlox METASPERSE™ (Croda International PLC, Edison, N.J.), BIO-SOFT® (e.g., N series, such as N1-3, Ni-?, N1-5, N1-9, N23-3, N2.3-6.5, N25-3, N25-7, N25-9, N91-2.5, N91-6, N91-8; Stepan Company, Northfield, Ill.), MAKON® nonionic surfactants (e.g., DA-4, DA-6 and DA-9; Stepan Company, Northfield, Ill.), MORWET® powders (Akzo Nobel Surface Chemistry LLC, Chicago, Ill.), MULTIWET™ surfactants (e.g., MO-85P-PW-(AP); Croda International PLC, Edison, N.J.), SILWET® L-77 (Helena Chemical Company, Collierville, Tenn.), SPAN™ surfactants (e.g., 20, 40, 60, 65, 80 and 85; Croda Inc., Edison N.J.), TAMOL™ dispersants (The Dow Chemical Company, Midland, Mich.), TERGITOL™ surfactants (e.g., TMN-6 and TMN-100X; The Dow Chemical Company, Midland, Mich.), TERSPERSE surfactants (e.g., 2001, 2020, 2100, 2105, 2158, 2700, 4894 and 4896; Hunstman Corp., The Woodlands, Tex.), TRITON™ surfactants (e.g., X-100; The Dow Chemical Company, Midland, Mich.), TWEEN® surfactants (e.g., TWEEN® 20, 21, 22, 23, 28, 40, 60, 61, 65, 80, 81 and 85; Croda International PLC, Edison, N.J.) and combinations thereof. Additional examples of dispersants may be found in Baird & Zublena. 1993. Soil Facts: Using wetting Agents (Nonionic Surfactants) on Soil (North Carolina Cooperative Extension Service Publication AG-439-25) (1993); Burges, composition of Microbial Biopesticides: Beneficial Microorganisms, Nematodes and Seed Treatments (Springer Science & Business Media) (2012); McCarty, Wetting Agents (Clemson University Cooperative Extension Service Publication) (2001).

In some embodiments, compositions of the present disclosure comprise one or more anionic surfactants. According to some embodiments, the composition comprises one or more water-soluble anionic surfactants and/or one or more water-insoluble anionic surfactants, optionally one or more anionic surfactants selected from the group consisting of alkyl carboxylates (e.g., sodium stearate), alkyl sulfates (e.g., alkyl lauryl sulfate, sodium lauryl sulfate), alkyl ether sulfates, alkyl amido ether sulfates, alkyl aryl polyether sulfates, alkyl aryl sulfates, alkyl aryl sulfonates, alkyl sulfonates, alkyl amide sulfonates, alkyl aryl sulfonates, alkyl benzene sulfonates, alkyl diphenyloxide sulfonate, alpha-olefin sulfonates, alkyl naphthalene sulfonates, paraffin sulfonates, alkyl sulfosuccinates, alkyl ether sulfosuccinates, alkylamide sulfosuccinates, alkyl sulfosuccinamates, alkyl sulfoacetates, alkyl phosphates, alkyl ether phosphates, acyl sarconsinates, acyl isethionates, N-acyl taurates, N-acyl-N-alkyltaurates, benzene sulfonates, cumene sulfonates, dioctyl sodium sulfosuccinate, ethoxylated sulfosuccinates, lignin sulfonates, linear alkylbenzene sulfonates, monoglyceride sulfates, perfluorobutanesulfonate, perfluorooctanesulfonate, phosphate ester, styrene acrylic polymers, toluene sulfonates and xylene sulfonates.

In some embodiments, compositions of the present disclosure comprise one or more cationic surfactants. According to some embodiments, the composition comprises one or more pH-dependent amines and/or one or more quaternary ammonium cations, optionally one or more cationic surfactants selected from the group consisting of alkyltrimethylammonium salts (e.g., cetyl trimethylammonium bromide, cetyl trimethylammonium chloride), cetylpyridinium chloride, benzalkonium chloride, benzethonium chloride, 5-Bromo-5-nitro-1,3-dioxane, dimethyldioctadecylammonium chloride, cetrimonium bromide, dioctadecyldimethylammonium bromide and/or octenidine dihydrochloride.

In some embodiments, compositions of the present disclosure comprise one or more nonionic surfactants. According to some embodiments, the composition comprises one or more water-soluble nonionic surfactants and/or one or more water-insoluble nonionic surfactants, optionally one or more nonionic surfactants selected from the group consisting of alcohol ethoxylates (e.g., TERGITOL™ 15-S surfactants, such as TERGITOL™ 15-S-9 (The Dow Chemical Company, Midland, Mich.)), alkanolamides, alkanolamine condensates, carboxylic acid esters, cetostearyl alcohol, cetyl alcohol, cocamide DEA, dodecyldimethylamine oxides, ethanolamides, ethoxylates of glycerol ester and glycol esters, ethylene oxide polymers, ethylene oxide-propylene oxide copolymers, glucoside alkyl ethers, glycerol alkyl ethers, glycerol esters, glycol alkyl ethers (e.g., polyoxyethylene glycol alkyl ethers, polyoxypropylene glycol alkyl ethers), glycol alkylphenol ethers (e.g., polyoxyethylene glycol alkylphenol ethers,), glycol esters, monolaurin, pentaethylene glycol monododecyl ethers, poloxamer, polyamines, polyglycerol polyricinoleate, polysorbate, polyoxyethylenated fatty acids, polyoxyethylenated mercaptans, polyoxyethylenated polyoxyproylene glycols, polyoxyethylene glycol sorbitan alkyl esters, polyethylene glycol-polypropylene glycol copolymers, polyoxyethylene glycol octylphenol ethers, polyvinyl pynolidones, sugar-based alkyl polyglycosides, sulfoanylamides, sorbitan fatty acid alcohol ethoxylates, sorbitan fatty acid ester ethoxylates, sorbitan fatty acid ester and/or tertiary acetylenic glycols.

In some embodiments, compositions of the present disclosure comprise at least one nonionic surfactant. According to some embodiments, the composition comprises at least one water insoluble nonionic surfactant and at least one water soluble nonionic surfactant. In some embodiments, compositions of the present disclosure comprise a combination of nonionic surfactants having hydrocarbon chains of substantially the same length.

In some embodiments, compositions of the present disclosure comprise one or more zwitterionic surfactants. According to some embodiments, the composition comprises one or more betaines and/or one or more sultaines, optionally one or more zwitterionic surfactants selected from the group consisting of 3-[(3-Cholamidopropyl)dimethylammonio]-1-propanesulfonate, cocamidopropyl betaine, cocamidopropyl hydroxysultaine, phosphatidylserine, phosphatidylethanolamine, phosphatidylcholine and/or one or more sphingomyelins.

In some embodiments, compositions of the present disclosure comprise one or more soaps and/or organosilicone surfactants. According to some embodiments, the composition comprises one or more alkali metal salts of fatty acids.

In some embodiments, compositions of the present disclosure comprise one or more wetting agents. According to some embodiments, the composition comprises one or more naphthalene sulfonates, optionally one or more alkyl naphthalene sulfonates (e.g., sodium alkyl naphthalene sulfonate), one or more isopropyl naphthalene sulfonates (e.g., sodium isopropyl naphthalene sulfonate) and/or one or more butyl naphthalene sulfonates (e.g., sodium n-butyl naphthalene sulfonate).

Compositions of the present disclosure may comprise any suitable drying agent(s), including, but not limited to, drying powders. Non-limiting examples of drying agents include AEROSIL® hydrophobic fumed silica powders (Evonik Corporation, Parsippany, N.J.), BENTOLITE® powders (BYK-Chemie GmbH, Wesel, Germany), INCOTEC® powders (INCOTEC Inc., Salinas, Calif.), SIPERNAT® silica powders (Evonik Corporation, Parsippany, N.J.) and combinations thereof. Additional examples of drying agents may be found in Burges, composition of Microbial Biopesticides: Beneficial Microorganisms, Nematodes and Seed Treatments (Springer Science & Business Media) (2012). In some embodiments, compositions of the present disclosure comprise calcium stearate, clay (e.g., attapulgite clay, montmorillonite clay), graphite, magnesium stearate, magnesium sulfate, powdered milk, silica (e.g., fumed silica, hydrophobically-coated silica, precipitated silica), soy lecithin and/or talc.

Compositions of the present disclosure may comprise any suitable anti-freezing agent(s), including, but not limited to, ethylene glycol, glycerin, propylene glycol and urea.

Compositions of the present disclosure may comprise any seed flowability agent to improve the lubricity of the treated seeds. The flowability agent may comprise one or more liquid lubricants, solid lubricants, liquid emulsions, or suspensions of solid lubricants. Non-limiting examples of flowability agents include, for example, lubricants such as fats and oils, natural and synthetic waxes, graphite, talc, fluoropolymers (e.g., polytetrafluoroethylene), and solid lubricants such as molybdenum disulfide and tungsten disulfide. In some instances, the flowability agent comprises a wax material. Non-limiting examples of wax materials that can be incorporated into the liquid seed treatment composition include plant and animal-derived waxes such as carnauba wax, candelilla wax, ouricury wax, beeswax, spermaceti, and petroleum derived waxes, such as paraffin wax. For example, in some instances, the flowability agent comprises carnauba wax. In some instances, the flowability agent comprises an oil. For example, the flowability agent may comprise soybean oil. Non-limiting examples of commercially available wax materials suitable for use as flowability agents include AQUAKLEAN 418 supplied by Micro Powders, Inc. (an anionic aqueous emulsion comprising extra light carnauba wax at 35% solids content).

Compositions of the present disclosure may comprise any suitable safener(s), including, but not limited to, napthalic anhydride.

Compositions of the present disclosure may comprise any suitable pH buffer(s), including, but not limited to, potassium phosphate monobasic and potassium phosphate dibasic. In some embodiments, the composition comprises one or more pH buffers selected to provide a composition having a pH of less than 10, typically from about 4.5 to about 9.5, from about 6 to about 8, or about 7.

Compositions of the present disclosure may comprise any suitable anti-settling agent(s), including, but not limited to, polyvinyl acetate, polyvinyl alcohols with different degrees of hydrolysis, polyvinylpyrrolidones, polyacrylates, acrylate-, polyol- or polyester-based paint system binders which are soluble or dispersible in water, moreover copolymers of two or more monomers such as acrylic acid, methacrylic acid, itaconic acid, maleic acid, fumaric acid, maleic anhydride, vinylpyrrolidone, ethylenically unsaturated monomers such as ethylene, butadiene, isoprene, chloroprene, styrene, divinylbenzene, ot-methylstyrene or p-methylstyrene, further vinyl halides such as vinyl chloride and vinylidene chloride, additionally vinyl esters such as vinyl acetate, vinyl propionate or vinyl stearate, moreover vinyl methyl ketone or esters of acrylic acid or methacrylic acid with monohydric alcohols or polyols such as methyl acrylate, methyl methacrylate, ethyl acrylate, ethylene methacrylate, lauryl acrylate, lauryl methacrylate, decyl acrylate, N,N-dimethylamino-ethyl methacrylate, 2-hydroxyethyl methacrylate, 2-hydroxypropyl methacrylate or glycidyl methacrylate, furthermore diethyl esters or monoesters of unsaturated dicarboxylic acids, furthermore (meth)acrylamido-N-methylol methyl ether, amides or nitriles such as acrylamide, methacrylamide, N-methylol(meth)acrylamide, acrylonitrile, methacrylonitrile, and also N-substituted maleiraides and ethers such as vinyl butyl ether, vinyl isobutyl ether or vinyl phenyl ether, and combinations thereof.

Compositions of the present disclosure may comprise any suitable adhesive(s), including, but not limited to, adhesive compositions comprising, consisting essentially of or consisting of one or more disaccharides (e.g. maltose), gums (e.g., cellulose gum, guar gum, gum arabic, gum combretum, xantham gum), maltodextrins (e.g., one or more maltodextrins (each and/or collectively) having a DEV of about 10 to about 20), monosaccharides, oils (e.g., mineral oil, olive oil, peanut oil, soybean oil and/or sunflower oil) and/or oligosaccharides.

Compositions of the present disclosure may comprise any suitable effect pigment(s). Effect pigments, which are sometimes also referred to in the art as “pearl pigments,” are a class of materials that provide reflectivity, shine, and/or a pearlescent effect when applied as a coating. In some instances, the effect pigment is in the form of a powder comprising a substrate material and a metal oxide coating. For example, the effect pigment may comprise a substrate material including but not limited to talc, silicate materials (e.g., mica), clay minerals, calcium carbonate, kaolin, phlogopite, alumina, and similar substances. In some instances, the substrate material comprises a hydrophilic material. The substrate material may be coated with a semi-transparent layer of a metal oxide, including but not limited to titanium dioxide, iron oxide, chromium oxide, or zirconium oxide. Alternatively, in some instances, the effect pigment comprises metal powder or metal flakes. The metal powder or metal flakes may comprise a metal including, but not limited to aluminum, copper, silver, or bronze. In some instances, the effect pigment comprises a silicate based substrate. Non-limiting examples of particulate silicates that can be incorporated into the dry powder coating include mica coated with titanium dioxide (e.g., SUNMICA FINE WHITE 2800102, which is commercially available from Sun Chemical Corp.). Other non-limiting examples of commercially available effect pigments that can be incorporated into the dry powder include MAGNA PEARL, LUMINA and MEARLIN pigments from BASF Corporation; PHIBRO PEARL from PhibroChem; and IRIDESIUM 120 from Aakash Chemicals. In some instances, the dry powder has a mean particle size of from about 1 to about 25 microns.

Compositions of the present disclosure may comprise any suitable growth medium suitable for culturing one or more of the microorganisms in the composition. For example, in some embodiments, compositions of the present disclosure comprise Czapek-Dox medium, glycerol yeast extract, mannitol yeast extract, potato dextrose broth and/or YEM media.

Carriers, stabilizing compounds, biostimulants, microbial extracts, nutrients, pest attractants and/or feeding stimulants, pesticides, plant signal molecules, dispersants, drying agents, safeners, flowability agents, anti-settling agents, buffers, adhesives, etc. may be incorporated into compositions of the present disclosure in any suitable amount(s)/concentration(s). The absolute value of the amount/concentration that is/are sufficient to cause the desired effect(s) may be affected by factors such as the type, size and volume of material to which the compositon will be applied, the type(s) of microorganisms in the composition, the number of microorganisms in the composition, the stability of the microorganisms in the composition and storage conditions (e.g., temperature, relative humidity, duration). Those skilled in the art will understand how to select effective amounts/concentrations using routine dose-response experiments. Guidance for the selection of appropriate amounts/concentrations can be found, for example, in International Patent Application Nos. PCT/US2016/050529 and PCT/US2016/050647 and U.S. Provisional Patent Application Nos. 62/296,798; 62/271,857; 62/347,773; 62/343,217; 62/296,784; 62/271,873; 62/347,785; 62/347,794; and 62/347,805.

In some embodiments, compositions of the present disclosure comprise one or more carriers in an amount/concentration of about 1 to about 99% or more (by weight, based upon the total weight of the composition). For example, compositions of the present disclosure may comprsise about 1, 2, 3, 4, 5, 6, 7, 8, 9,10, 15, 20, 25, 30, 35, 40, 45, 50, 55, 60, 65, 70, 75, 80, 85, 90, 91, 92, 93, 94, 95, 96, 97, 98 or 99% (by weight) of one or more non-aqueous carriers.

In some embodiments, compositions of the present disclosure comprise one or more stabilizing compounds in an amount/concentration of about 0.0001 to about 95% or more (by weight, based upon the total of the composition). For example, compositions of the present disclosure may comprise about 0.0001 to about 0.001, about 0.001 to about 1%, about 0.25 to about 5%, about 1 to about 10%, about 5 to about 25%, about 10% to about 30%, about 20% to about 40%, about 25% to about 50%, about 30 to about 60%, about 50 to about 75%, or about 75 to about 95% (by weight), optionally about 0.0005, 0.001, 0.002, 0.003, 0.004, 0.005, 0.0075, 0.01, 0.02, 0.03, 0.04, 0.05. 0.06, 0.07, 0.08, 0.09, 0.1, 0.2, 0.3, 0.4, 0.5, 1, 1.5, 2, 2.5, 3, 3.5, 4, 4.5, 5, 5.5, 6, 6.5, 7, 7.5, 8, 8.5, 9, 9.5, 10, 15, 20, 25, 30, 35, 40, 45, 50, 55, 60, 65, 70, 75, 80, 85, 90, 95%, of one or more maltodextrins, monosaccharides, disaccharides, sugar alcohols, humic acids, betaines, prolines, sarcosines, peptones, oxidation control components, hygroscopic polymers and/or UV protectants.

In some embodiments, compositions of the present disclosure comprise one or more stabilizing compounds at a concentration of about 1×10⁻²⁰ M to about 1×10⁻¹ M. For example, compositions of the present disclosure may comprise about 1×10⁻¹⁵ M to about 1×10⁻¹⁰ M, about 1×10⁻¹⁴ M to about 1×10⁻⁸ M, about 1×10⁻¹⁴ M to about 1×10⁻⁶ M, about 1×10⁻¹² M to about 1×10⁻⁸ M, about 1×10⁻¹² M to about 1×10⁻⁶ M, about 1×10⁻¹⁰ M to about 1×10⁻⁶ M, or about 1×10⁻⁸ M to about 1×10⁻² M, optionally about 1×10⁻²⁰ M, 1×10⁻¹⁹ M, 1×10⁻¹⁸ M, 1×10⁻¹⁷ M, 1×10⁻¹⁶ M, 1×10⁻¹⁵ M, 1×10⁻¹⁴ M, 1×10⁻¹³ M, 1×10⁻¹² M, 1×10⁻¹¹ M, 1×10⁻¹⁰ M, 1×10⁻⁹ M, 1×10⁻⁸ M, 1×10⁻⁷ M, 1×10⁻⁶ M, 1×10⁻⁵ M, 1×10⁻⁴ M, 1×10⁻³ M, 1×10⁻² M, 1×10⁻¹ M or more, of one or more maltodextrins, monosaccharides, disaccharides, sugar alcohols, humic acids, betaines, prolines, sarcosines, peptones, oxidation control components, hygroscopic polymers and/or UV protectants.

In some embodiments, compositions of the present disclosure comprise one or more monosaccharides in an amount/concentration of about 0.005 to about 50% (by weight) of the composition. For example, compositions of the present disclosure may comprise about/at least/less than 0.01, 0.02, 0.03, 0.04, 0.05, 0.06, 0.07, 0.08, 0.09, 0.1, 0.15, 0.2, 0.25, 0.3, 0.35, 0.4, 0.45, 0.5, 0.75, 1, 1.25, 1.5, 2, 2.5, 3, 3.5, 4, 4.5, 5, 5.5, 6, 6.5, 7, 7.5, 8, 8.5, 9, 10, 15, 20, 25% (by weight) of one or more monosaccharides (e.g., arabinose, fructose and/or glucose). In some embodiments, one or more monosaccharides is/are present in a concentration ranging from about 1×10⁻²⁰ M to about 1×10⁻¹ M. For example, one or more monosaccharides may be included at a concentration of about/at least/less than 1×10⁻²⁰ M, 1×10⁻¹⁹ M, 1×10⁻¹⁸ M, 1×10⁻¹⁷ M, 1×10⁻¹⁶ M, 1×10⁻¹⁵ M, 1×10⁻¹⁴ M, 1×10⁻¹³ M, 1×10⁻¹² M, 1×10⁻¹¹ M, 1×10⁻¹⁰ M.

In some embodiments, compositions of the present disclosure comprise one or more disaccharides in an amount/concentration of about 0.005 to about 50% (by weight) of the composition. For example, compositions of the present disclosure may comprise about/at least/less than 0.01, 0.02, 0.03, 0.04, 0.05, 0.06, 0.07, 0.08, 0.09, 0.1, 0.15, 0.2, 0.25, 0.3, 0.35, 0.4, 0.45, 0.5, 0.75, 1, 1.25, 1.5, 2, 2.5, 3, 3.5, 4, 4.5, 5, 5.5, 6, 6.5, 7, 7.5, 8, 8.5, 9, 10, 15, 20, 25% (by weight) of one or more disaccharides (e.g., maltose, sucrose and/or trehalose). In some embodiments, one or more disaccharides is/are present in a concentration ranging from about 1×10⁻²⁰ M to about 1×10⁻¹ M. For example, one or more disaccharides may be included at a concentration of about/at least/less than 1×10⁻²⁰ M, 1×10⁻¹⁹ M, 1×10⁻¹⁸ M, 1×10⁻¹⁷ M, 1×10⁻¹⁶ M, 1×10⁻¹⁵ M, 1×10⁻¹⁴ M, 1×10⁻¹³ M, 1×10⁻¹² M, 1×10⁻¹¹ M, 1×10⁻¹⁰ M.

In some embodiments, compositions of the present disclosure comprise one or more maltodextrins in an amount/concentration of about 0.001 to about 95% or more (by weight) of the composition. In some embodiments, the maltodextrin(s) comprise(s) about 0.001 to about 1%, about 0.25 to about 5%, about 1 to about 10%, about 5 to about 25%, about 10% to about 30%, about 20% to about 40%, about 25% to about 50%, about 50 to about 75%, or about 75 to about 95% (by weight) of the composition. For example, compositions of the present disclosure may comprise about/at least/less than 0.01, 0.02, 0.03, 0.04, 0.05. 0.06, 0.07, 0.08, 0.09, 0.1, 0.2, 0.3, 0.4, 0.5, 1, 1.5, 2, 2.5, 3, 3.5, 4, 4.5, 5, 5.5, 6, 6.5, 7, 7.5, 8, 8.5, 9, 9.5, 10, 15, 20, 25, 30, 35, 40, 45, 50, 55, 60, 65, 70, 75, 80, 85, 90, 95% or more (by weight) of one or more maltodextrins (e.g., one or more maltodextrins (each and/or collectively) having a DEV value of about 15 to about 20).

In some embodiments, compositions of the present disclosure comprise one or more sugar alcohols in an amount/concentration of about 0.001 to about 95% or more (by weight) of the composition. In some embodiments, the sugar alcohol(s) (e.g., arabitol, mannitol, sorbitol and/or xylitol) comprise(s) about 0.001 to about 1%, about 0.25 to about 5%, about 1 to about 10%, about 5 to about 25%, about 10% to about 30%, about 20% to about 40%, about 25% to about 50%, about 50 to about 75%, or about 75 to about 95% (by weight) of the composition. For example, compositions of the present disclosure may comprise about/at least/less than 0.01, 0.02, 0.03, 0.04, 0.05. 0.06, 0.07, 0.08, 0.09, 0.1, 0.2, 0.3, 0.4, 0.5, 1, 1.5, 2, 2.5, 3, 3.5, 4, 4.5, 5, 5.5, 6, 6.5, 7, 7.5, 8, 8.5, 9, 9.5, 10, 15, 20, 25, 30, 35, 40, 45, 50, 55, 60, 65, 70, 75, 80, 85, 90, 95% or more (by weight) of one or more sugar alcohols (e.g., arabitol, mannitol, sorbitol and/or xylitol).

In some embodiments, compositions of the present disclosure comprise one or more humic acids in an amount/concentration of about 0.001 to about 95% or more (by weight) of the composition. In some embodiments, the humic acid(s) (e.g., potassium humate) comprise(s) about 0.001 to about 1%, about 0.25 to about 5%, about 1 to about 10%, about 5 to about 25%, about 10% to about 30%, about 20% to about 40%, about 25% to about 50%, about 50 to about 75%, or about 75 to about 95% (by weight) of the composition. For example, compositions of the present disclosure may comprise about/at least/less than 0.01, 0.02, 0.03, 0.04, 0.05. 0.06, 0.07, 0.08, 0.09, 0.1, 0.2, 0.3, 0.4, 0.5, 1, 1.5, 2, 2.5, 3, 3.5, 4, 4.5, 5, 5.5, 6, 6.5, 7, 7.5, 8, 8.5, 9, 9.5, 10, 15, 20, 25, 30, 35, 40, 45, 50, 55, 60, 65, 70, 75, 80, 85, 90, 95% or more (by weight) of one or more humic acids (e.g., potassium humate and/or sodium humate).

In some embodiments, compositions of the present disclosure comprise one or more UV protectants in an amount/concentration of about 0.0001 to about 5% or more (by weight) of the composition. In some embodiments, the UV protectant(s) (e.g., calcium lignosulfate and/or sodium lignosulfate) comprise(s) about 0.0001 to about 0.001, about 0.001 to about 1%, about 0.25 to about 5%, (by weight) of the composition. For example, compositions of the present disclosure may comprise about/at least/less than 0.0005, 0.001, 0.002, 0.003, 0.004, 0.005, 0.0075, 0.01, 0.02, 0.03, 0.04, 0.05, 0.06, 0.07, 0.08, 0.09, 0.1, 0.15, 0.2, 0.25, 0.3, 0.35, 0.4, 0.45, 0.5, 0.75, 1, 1.25, 1.5, 2, 2.5, 3, 3.5, 4, 4.5, 5% or more (by weight) of one or more UV protectants (e.g., calcium lignosulfate and/or sodium lignosulfate).

In some embodiments, compositions of the present disclosure comprise one or more oxidation control components in an amount/concentration of about 0.0001 to about 5% or more (by weight) of the composition. For example, compositions of the present disclosure may comprise about/at least/less than 0.0005, 0.001, 0.002, 0.003, 0.004, 0.005, 0.0075, 0.01, 0.02, 0.03, 0.04, 0.05, 0.06, 0.07, 0.08, 0.09, 0.1, 0.15, 0.2, 0.25, 0.3, 0.35, 0.4, 0.45, 0.5, 0.75, 1, 1.25, 1.5, 2, 2.5, 3, 3.5, 4, 4.5, 5% of one or more oxidation control components. In some embodiments, the amount/concentration of oxidation control components is about 0.005 to about 2% (by weight) of the composition. In some embodiments, the oxidation control component(s) is/are present in a concentration ranging from about 1×10⁻²⁰ M to about 1×10⁻¹ M. For example, one or more oxidation control components may be added at a concentration of about/at least/less than 1×10⁻²⁰ M, 1×10⁻¹⁹ M, 1×10⁻¹⁸ M, 1×10⁻¹⁷ M, 1×10⁻¹⁶ M, 1×10⁻¹⁵ M, 1×10⁻¹⁴ M, 1×10⁻¹³ M, 1×10⁻¹² M, 1×10⁻¹¹ M, 1×10⁻¹⁰ M. In some embodiments, compositions of the present disclosure comprise one or more commercial antioxidants used in accordance with the manufacturer's recommended amounts/concentrations. In some embodiments, compositions of the present disclosure comprise one or more commercial oxygen scavengers used in accordance with the manufacturer's recommended amounts/concentrations.

In some embodiments, compositions of the present disclosure comprise one or more stabilizing compounds in an amount/concentration sufficient to ensure Yersinia/Bacillus remain viable.

In some embodiments, compositions of the present disclosure comprise one or more stabilizing compounds in an amount/concentration sufficient to ensure the deliquescence relative humidity (DRH) of the composition is less than 5, 10, 15, 20, 25, 30, 35, 40, 45, 50, 55, 60, 65, 70, 75, 80, 85 or 90 at the temperature(s) at which the composition is to be stored (e.g., 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 and/or 40° C.).

Stablizing compounds may be incorporated into compositions of the present disclosure in any suitable ratio(s).

In some embodiments, compositions of the present disclosure comprise one or more maltodextrins and one or more monosaccharides, disaccharides, sugar alcohols and/or humic acids in a maltodextrin:(monosaccharide, disaccharide, sugar alcohol and/or humic acid) ratio of about 5:95, 10:90, 15:85, 20:80, 25:75, 30:70, 35:65, 40:60, 45:55, 50:50, 55:45, 60:40, 65:35, 70:30, 75:25, 80:20, 85:15, 90:10, 95:5. For example, compositions of the present disclosure may comprise one or more maltodextrins (e.g., one or more maltodextrins (each and/or collectively) having a DEV of about 15 to about 20) and one or more sugar alcohols (e.g., sorbitol and/or xylitol) and/or humic acids (e.g., potassium humate) in a maltodextrin:(sugar alcohol/humic acid) ratio of about 5:95, about 15:85, about 25:75 or about 50:50.

In some embodiments, compositions of the present disclosure comprise one or more microbial extracts in an amount/concentration of about 0.0001 to about 5% or more (by weight) of the composition. In some embodiments, the microbial extract(s) comprise(s) about 0.0001, 0.0002, 0.0003, 0.0004, 0.0005, 0.0006, 0.0007, 0.0008, 0.0009, 0.001, 0.0015, 0.002, 0.0025, 0.003, 0.0035, 0.004, 0.0045, 0.005, 0.0055, 0.006, 0.0065, 0.007, 0.0075, 0.008, 0.0085, 0.009, 0.0095, 0.01, 0.015, 0.02, 0.025, 0.03, 0.035, 0.04, 0.045, 0.05, 0.06, 0.07, 0.08, 0.09, 0.1, 0.02, 0.3, 0.4, 0.5, 0.6, 0.7, 0.8, 0.9, 1 to about 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4., 4.5, 4.6, 4.7, 4.8, 4.9, 5% (by weight) of the composition. For example, compositions of the present disclosure may comprise about 0.0005, 0.00075, 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.15, 0.2, 0.25, 0.3, 0.35, 0.4, 0.45, 0.5, 0.55, 0.6, 0.65, 0.7, 0.75, 0.8, 0.85, 0.9, 0.95, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4., 4.5, 4.6, 4.7, 4.8, 4.9, 5% or more (by weight) of one or more microbial extracts.

In some embodiments, compositions of the present disclosure comprise one or more nutrients in an amount/concentration of about 0.0001 to about 5% or more (by weight) of the composition. In some embodiments, the nutrient(s) (e.g., phosphorous, boron, chlorine, copper, iron, manganese, molybdenum and/or zinc) comprise(s) about 0.0001, 0.0002, 0.0003, 0.0004, 0.0005, 0.0006, 0.0007, 0.0008, 0.0009, 0.001, 0.0015, 0.002, 0.0025, 0.003, 0.0035, 0.004, 0.0045, 0.005, 0.0055, 0.006, 0.0065, 0.007, 0.0075, 0.008, 0.0085, 0.009, 0.0095, 0.01, 0.015, 0.02, 0.025, 0.03, 0.035, 0.04, 0.045, 0.05, 0.06, 0.07, 0.08, 0.09, 0.1, 0.02, 0.3, 0.4, 0.5, 0.6, 0.7, 0.8, 0.9, 1 to about 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4., 4.5, 4.6, 4.7, 4.8, 4.9, 5% (by weight) of the composition. For example, compositions of the present disclosure may comprise about 0.0005, 0.00075, 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.15, 0.2, 0.25, 0.3, 0.35, 0.4, 0.45, 0.5, 0.55, 0.6, 0.65, 0.7, 0.75, 0.8, 0.85, 0.9, 0.95, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4., 4.5, 4.6, 4.7, 4.8, 4.9, 5% or more (by weight) of one or more the nutrients (e.g., phosphorous, boron, chlorine, copper, iron, manganese, molybdenum and/or zinc).

In some embodiments, compositions of the present disclosure comprise one or more pest attractant(s) and/or feeding stimulant(s) in an amount/concentration of about 0.0001 to about 5% or more (by weight) of the composition. In some embodiments, the pest attractant(s) and/or feeding stimulant(s) comprise(s) about 0.0001, 0.0002, 0.0003, 0.0004, 0.0005, 0.0006, 0.0007, 0.0008, 0.0009, 0.001, 0.0015, 0.002, 0.0025, 0.003, 0.0035, 0.004, 0.0045, 0.005, 0.0055, 0.006, 0.0065, 0.007, 0.0075, 0.008, 0.0085, 0.009, 0.0095, 0.01, 0.015, 0.02, 0.025, 0.03, 0.035, 0.04, 0.045, 0.05, 0.06, 0.07, 0.08, 0.09, 0.1, 0.02, 0.3, 0.4, 0.5, 0.6, 0.7, 0.8, 0.9, 1 to about 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4., 4.5, 4.6, 4.7, 4.8, 4.9, 5% (by weight) of the composition. For example, compositions of the present disclosure may comprise about 0.0005, 0.00075, 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.15, 0.2, 0.25, 0.3, 0.35, 0.4, 0.45, 0.5, 0.55, 0.6, 0.65, 0.7, 0.75, 0.8, 0.85, 0.9, 0.95, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4., 4.5, 4.6, 4.7, 4.8, 4.9, 5% or more (by weight) of one or more pest attractants and/or feeding stimulants.

In some embodiments, compositions of the present disclosure comprise one or more dispersants in an amount/concentration of about 0.001 to about 25% or more (by weight) of the composition. In some embodiments, the dispersant(s) comprise(s) 0.001, 0.0015, 0.002, 0.0025, 0.003, 0.0035, 0.004, 0.0045, 0.005, 0.0055, 0.006, 0.0065, 0.007, 0.0075, 0.008, 0.0085, 0.009, 0.0095, 0.01, 0.015, 0.02, 0.025, 0.03, 0.035, 0.04, 0.045, 0.05, 0.06, 0.07, 0.08, 0.09, 0.1, 0.02, 0.3, 0.4, 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4., 4.5, 4.6, 4.7, 4.8, 4.9, 5, 6, 7, 8, 9 or 10 to about 1, 1.5, 2, 2.5, 3, 3.5, 4, 4.5, 5, 5.5, 6, 6.5, 7, 7.5, 8, 8.5, 9, 9.5, 10, 11, 12, 13, 14, 15, 16, 17, 18, 19 or 20% (by weight) of the composition. For example, compositions of the present disclosure may comprise about 0.01, 0.02, 0.03, 0.04, 0.05. 0.06, 0.07, 0.08, 0.09, 0.1, 0.2, 0.3, 0.4, 0.5, 1, 1.5, 2, 2.5, 3, 3.5, 4, 4.5, 5, 5.5, 6, 6.5, 7, 7.5, 8, 8.5, 9, 9.5, 10, 15, 20% or more (by weight) of one or more dispersants (e.g., one or more surfactants and/or wetting agents).

In some embodiments, compositions of the present disclosure comprise one or more drying agents in an amount/concentration of about 0.001 to about 95% or more (by weight) of the composition. In some embodiments, the drying agent(s) comprise(s) about) 0.001, 0.0015, 0.002, 0.0025, 0.003, 0.0035, 0.004, 0.0045, 0.005, 0.0055, 0.006, 0.0065, 0.007, 0.0075, 0.008, 0.0085, 0.009, 0.0095, 0.01, 0.015, 0.02, 0.025, 0.03, 0.035, 0.04, 0.045, 0.05, 0.06, 0.07, 0.08, 0.09, 0.1, 0.02, 0.3, 0.4, 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4., 4.5, 4.6, 4.7, 4.8, 4.9, 5, 6, 7, 8, 9 or 10 to about 1, 1.5, 2, 2.5, 3, 3.5, 4, 4.5, 5, 5.5, 6, 6.5, 7, 7.5, 8, 8.5, 9, 9.5, 10, 11, 12, 13, 14, 15, 16, 17, 18, 19 or 20% (by weight) of the composition. For example, compositions of the present disclosure may comprise about 0.01, 0.02, 0.03, 0.04, 0.05. 0.06, 0.07, 0.08, 0.09, 0.1, 0.2, 0.3, 0.4, 0.5, 1, 1.5, 2, 2.5, 3, 3.5, 4, 4.5, 5, 5.5, 6, 6.5, 7, 7.5, 8, 8.5, 9, 9.5, 10, 15, 20, 25, 30, 35, 40, 45, 50, 55, 60, 65, 70, 75, 80, 85, 90, 95% or more (by weight) of one or more drying agents (e.g., lecithin and/or talc). In some embodiments, the compositions of the present disclosure comprise about 0.5 to about 10 grams of drying powder per liter of composition. For example, compositions of the present disclosure may comprise about 0.5, 1, 1.25, 1.5, 1.75, 2, 2.25, 2.5, 2.75, 3, 3.25, 3.5, 3.75, 4, 4.25, 4.5, 4.75, 5, 5.5, 6, 6.5, 7, 7.5, 8, 8.5, 9, 9.5, 10 grams or more of drying powder per liter of composition.

In some embodiments, compositions of the present disclosure comprise one or more buffers in an amount/concentration of about 0.0001 to about 5% or more (by weight) of the composition. In some embodiments, the buffer(s) comprise(s) about 0.0001, 0.0002, 0.0003, 0.0004, 0.0005, 0.0006, 0.0007, 0.0008, 0.0009, 0.001, 0.0015, 0.002, 0.0025, 0.003, 0.0035, 0.004, 0.0045, 0.005, 0.0055, 0.006, 0.0065, 0.007, 0.0075, 0.008, 0.0085, 0.009, 0.0095, 0.01, 0.015, 0.02, 0.025, 0.03, 0.035, 0.04, 0.045, 0.05, 0.06, 0.07, 0.08, 0.09, 0.1, 0.02, 0.3, 0.4, 0.5, 0.6, 0.7, 0.8, 0.9, 1 to about 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4., 4.5, 4.6, 4.7, 4.8, 4.9, 5% (by weight) of the composition. For example, compositions of the present disclosure may comprise about 0.0005, 0.00075, 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.15, 0.2, 0.25, 0.3, 0.35, 0.4, 0.45, 0.5, 0.55, 0.6, 0.65, 0.7, 0.75, 0.8, 0.85, 0.9, 0.95, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4., 4.5, 4.6, 4.7, 4.8, 4.9, 5% or more (by weight) of one or more buffers (e.g., potassium phosphate monobasic and/or potassium phosphate dibasic).

In some embodiments, compositions of the present disclosure comprise one or more commercial carriers, antioxidants, oxygen scavengers, hygroscopic polymers, UV protectants, biostimulants, microbial extracts, nutrients, pest attractants and/or feeding stimulants, pesticides, plant signal molecules, disperants, drying agents, anti-freezing agents, buffers and/or adhesives used in accordance with the manufacturer's recommended amounts/concentrations.

Compositions of the present disclosure may be formulated as any suitable type of composition, including, but not limited to, foliar compositions, seed coatings and soil composition.

In some embodiments, compositions of the present disclosure are formulated as amorphous solids. In some embodiments, compositions of the present disclosure are formulated as amorphous liquids. In some embodiments, compositions of the present disclosure are formulated as wettable powders.

In some embodiments, compositions of the present disclosure are formulated as liquid compositions that are subsequently dried to produce a powder or granule. For example, in some embodiments, liquid compositions of the present disclosure are drum dried, evaporation dried, fluidized bed dried, freeze dried, spray dried, spray-freeze dried, tray dried and/or vacuum dried to produce powders/granules. Such powders/granules may be further processed using any suitable method(s), including, but not limited to, flocculation, granulation and milling, to achieve a desired particle size or physical format. The precise method(s) and parameters of processing dried powders/granules that are appropriate in a given situation may be affected by factors such as the desired particle size(s), the type, size and volume of material to which the composition will be applied, the type(s) of microorganisms in the composition, the number of microorganisms in the composition, the stability of the microorganisms in the composition and the storage conditions (e.g., temperature, relative humidity, duration). Those skilled in the art will understand how to select appropriate methods and parameters using routine experiments.

In some embodiments, compositions of the present disclosure are frozen for cryopreservation. For example, in some embodiments, liquid compositions of the present disclosure are flash-frozen and stored in a cryopreservation storage unit/facility. The precise method(s) and parameters of freezing and preserving compositions of the present disclosure that are appropriate in a given situation may be affected by factors such as the type(s) of microorganisms in the composition, the number of microorganisms in the composition, the stability of the microorganisms in the composition and the storage conditions (e.g., temperature, relative humidity, duration). Those skilled in the art will understand how to select appropriate methods and parameters using routine experiments.

Compositions of the present disclosure may be formulated as aqueous or non-aqueous compositions. In some embodiments, compositions of the present disclosure comprise no water. In some embodiments, compositions of the present disclosure comprise a trace amount of water. In some embodiments, compositions of the present disclosure comprise less than 0.01, 0.02, 0.03, 0.04, 0.05, 0.06, 0.07, 0.08, 0.09, 0.1, 0.15, 0.2, 0.25, 0.3, 0.35, 0.4, 0.45, 0.5, 0.55, 0.6, 0.65, 0.7, 0.75 0.8, 0.85, 0.9, 0.95, 1, 1.25, 1.5, 1.75, 2, 2.25, 2.5, 2.75, 3, 3.25, 3.5, 3.75, 4, 4.25, 4.5, 4.75 or 5% water by weight, based upon the total weight of the composition.

In some embodiments, compositions of the present disclosure are formulated to have a pH of about 4.5 to about 9.5. In some embodiments, compositions of the present disclosure have a pH of about 6 to about 7.5. In some embodiments, compositions of the present disclosure have a pH of about 5, 5.5, 6, 6.5, 7, 7.5, 8 or 8.5.

Compositions of the present disclosure may contain a variety of carriers, stabilizers, nutrients, pesticides, plant signal molecules, dispersants, etc. It is to be understood that the components to be included in the composition and the order in which components are incorporated into the composition may be chosen or designed to maintain or enhance the dispersion, stability and/or survival of Yersinia bacteria during storage, distribution, and/or application of the composition.

It is to be understood that compositions of the present disclosure are non-naturally occurring compositions. According to some embodiments, the composition comprises one or more non-naturally occurring components. According to some embodiments, the composition comprises a non-naturally occurring combination of naturally occurring components.

Yersinia and Bacillus thuringiensis may be applied to any plant type, including, but not limited to, row crops and vegetables. In some embodiments, the compositions of the present disclosure are formulated for the treatment of one or more plants selected from the families Amaranthaceae (e.g., chard, spinach, sugar beet, quinoa), Asteraceae (e.g., artichoke, asters, chamomile, chicory, chrysanthemums, dahlias, daisies, echinacea, goldenrod, guayule, lettuce, marigolds, safflower, sunflowers, zinnias), Brassicaceae (e.g., arugula, broccoli, bok choy, Brussels sprouts, cabbage, cauliflower, canola, collard greens, daikon, garden cress, horseradish, kale, mustard, radish, rapeseed, rutabaga, turnip, wasabi, watercress, Arabidopsis thaliana), Cucurbitaceae (e.g., cantaloupe, cucumber, honeydew, melon, pumpkin, squash (e.g., acorn squash, butternut squash, summer squash), watermelon, zucchini), Fabaceae (e.g., alfalfa, beans, carob, clover, guar, lentils, mesquite, peas, peanuts, soybeans, tamarind, tragacanth, vetch), Malvaceae (e.g., cacao, cotton, durian, hibiscus, kenaf, kola, okra), Poaceae (e.g., bamboo, barley, corn, fonio, lawn grass (e.g., Bahia grass, Bermudagrass, bluegrass, Buffalograss, Centipede grass, Fescue, or Zoysia), millet, oats, ornamental grasses, rice, lye, sorghum, sugar cane, triticale, wheat and other cereal crops, Polygonaceae (e.g., buckwheat), Rosaceae (e.g., almonds, apples, apricots, blackberry, blueberry, cherries, peaches, plums, quinces, raspberries, roses, strawberries), Solanaceae (e.g., bell peppers, chili peppers, eggplant, petunia, potato, tobacco, tomato) and Vitaceae (e.g., grape). In some embodiments, the compositions of the present disclosure are formulated for the treatment of one or more plants with which Yersinia/Bacillus is not naturally associated (e.g., one or more plants that does not naturally exist in the geographical location(s) from which Yersinia/Bacillus was isolated). In some embodiments, the compositions of the present disclosure are formulated for the treatment of one or more acaricide-, fungicide-, gastropodicide-, herbicide-, insecticide-, nematicide-, rodenticide- and/or virucide-resistant plants (e.g., one or more plants resistant to acetolactate synthase inhibitors (e.g., imidazolinone, pryimidinyoxy(thio)benzoates, sulfonylaminocarbonyltriazolinone, sulfonylurea, triazolopyrimidines), bialaphos, glufosinate, glyphosate, hydroxyphenylpyruvatedioxygenase inhibitors and/or phosphinothricin). Non-limiting examples of plants that may be treated with compositions of the present disclosure include plants sold by Monsanto Company (St. Louis, Mo.) under the BOLLGARD II®, DROUGHTGARD®, GENUITY®, RIB COMPLETE®, ROUNDUP READY®, ROUNDUP READY 2 YIELD®, ROUNDUP READY 2 EXTEND™, SMARTSTAX®, VT DOUBLE PRO®, VT TRIPLE PRO®, YIELDGARD®, YIELDGARD VT ROOTWORM/RR2®, YIELDGARD VT TRIPLE® and/or XTENDFLEX™ tradenames.

The compositions of the present disclosure may be applied to any part/portion of a plant. In some embodiments, the compositions are applied to plant propagation materials (e.g., cuttings, rhizomes, seeds and tubers). In some embodiments, the compositions are applied to the roots of a plant. In some embodiments, the compositions are applied to the foliage of a plant. In some embodiments, the compositions are applied to both the roots and the foliage of a plant. In some embodiments, the compositions are applied to plant propagation materials and to the plants that grow from said plant propagation materials.

The compositions of the present disclosure may be applied to any plant growth medium, including, but not limited to, soil.

The compositions of the present disclosure may be applied to plants, plant parts and/or plant growth media in any suitable manner, including, but not limited to, on-seed application, in-furrow application and foliar application.

The compositions of the present disclosure may be applied using any suitable method(s), including, but not limited to, coating, dripping, dusting, encapsulating, immersing, spraying and soaking. Batch systems, in which predetermined batch sizes of material and composition are delivered into a mixer, may be employed. Continuous treatment systems, which are calibrated to apply composition at a predefined rate in proportion to a continuous flow of material, may also be employed.

In some embodiments, the compositions are applied directly to plant propagation material (e.g., seeds). According to some embodiments, plant propagation materials are soaked in a composition comprising the compositions for at least 0.1, 0.2, 0.3, 0.4, 0.5, 0.6, 0.7, 0.8, 0.9, 1, 1.25, 1.5, 1.75, 2, 3, 4, 5, 6, 9, 12, 15, 18, 21, 24, 36, 48 hours. According to some embodiments, plant propagation materials are coated with the compositions. Plant propagation materials may be coated with one or more additional layers (e.g., one or more protective layers that serve to enhance the stability and/or survival of Yersinia/Bacillus and/or one or more sequestration layers comprising substances that may reduce the stability and/or survival of Yersinia if included in the same layer as Yersinia/Bacillus). In some embodiments, the coating comprises, consists essentially of, or consists of a composition of the present disclosure and a drying powder.

In some embodiments, the compositions are applied directly to a plant growth medium (e.g., a soil). According to some embodiments, the compositions are applied in the vicinity of a plant propagation material (e.g., a seed). According to some embodiments, the compositions are applied to the root zone of a plant. According to some embodiments, the compositions are applied using a drip irrigation system.

In some embodiments, the compositions are applied directly to plants. According to some embodiments, the compositions are sprayed and/or sprinkled on the plant(s) to be treated.

In some embodiments, foliar application (e.g., application to leaves) of the compositions are used. Individual components of the compositions (e.g., Yersinia/Bacillus) may be separately applied by foliar means, or they may be applied together. Combinations of some components of the compositions may be separately applied by foliar means. All components of the compositions may be applied by foliar means.

In some embodiments, one of the microbes (Yersinia or Bacillus) may be applied to one part of a plant (e.g., seed coating) and the other microbe may be applied to another part of the plant (e.g., foliar application to leaves). In some embodiments, the two microbes may be applied at the same time, or may be applied at different times during the life cycle of the plant (e.g., seeds first and leaves subsequently).

In some embodiments, the compositions are freeze-spray- or spray-freeze-dried and then applied to plants/plant parts. For examples, in some embodiments, a composition comprising the compositions and one or more stabilizing components (e.g., one or more maltodextrins having a DEV of about 15 to about 20) is freeze-spray- or spray-freeze-dried, mixed with a drying powder (e.g., a drying powder comprising calcium stearate, attapulgite clay, montmorillonite clay, graphite, magnesium stearate, silica (e.g., fumed silica, hydrophobically-coated silica and/or precipitated silica) and/or talc), then coated on seed that was been pre-treated with one or more adhesives (e.g., an adhesive composition comprising one or more maltodextrins, one or more mono-, di- or oligosaccharides, one or more peptones, etc.), one or more pesticides and/or one or more plant signal molecules (e.g., one or more LCDs).

The compositions of the present disclosure may be applied to plants, plant parts and/or plant growth media in any suitable amount(s)/concentration(s).

In some embodiments, the compositions are applied at a rate of about 1×10¹ to about 1×10²⁰ CFU per kilogram of plant propagation material. According to some embodiments, the compositions are applied in an amount sufficient to ensure the plant propagation materials are coated with about/at least 1×10⁴, 1×10⁵, 1×10⁶, 1×10⁷, 1×10⁸, 1×10⁹, 1×10¹⁰, 1×10¹¹, 1×10¹², 1×10¹³, 1×10¹⁴, 1×10¹⁵ CFU of Yersinia/Bacillus per kilogram of plant propagation material. According to some embodiments, one or more microbial strains of the present disclosure is/are applied in an amount sufficient to ensure that an average of about/at least 1×10³, 1×10⁴, 1×10⁵, 1×10⁶, 1×10⁷, 1×10⁸, 1×10⁹, 1×10¹⁰, 1×10¹¹, 1×10¹², 1×10¹³, 1×10¹⁴, 1×10¹⁵ CFU of Yersinia/Bacillus is applied to each seed.

In some embodiments, the composition is applied at a rate of about 1×10¹ to about 1×10²⁰ CFU per plant. According to some embodiments, one or more microbial strains of the present disclosure is/are applied in an amount sufficient to ensure each plant is treated with about/at least 1×10⁴, 1×10⁵, 1×10⁶, 1×10⁷, 1×10⁸, 1×10⁹, 1×10¹⁰, 1×10¹¹, 1×10¹², 1×10¹³, 1×10¹⁴, 1×10¹⁵ CFU of Yersinia/Bacillus. According to some embodiments, Yersinia/Bacillus is applied in an amount sufficient to ensure that an average of about/at least 1×10³, 1×10⁴, 1×10⁵, 1×10⁶, 1×10⁷, 1×10⁸, 1×10⁹, 1×10¹⁰, 1×10¹¹, 1×10¹², 1×10¹³, 1×10¹⁴, 1×10¹⁵ CFU of Yersinia/Bacillus are applied to each plant.

In some embodiments, the compositions are applied at a rate of about 1×10¹ to about 1×10²⁰ CFU per acre of treated crops. According to some embodiments, Yersinia/Bacillus is applied in an amount sufficient to ensure each acre of treated crops is treated with about/at least 1×10⁴, 1×10⁵, 1×10⁶, 1×10⁷, 1×10⁸, 1×10⁹, 1×10¹⁰, 1×10¹¹, 1×10¹², 1×10¹³, 1×10¹⁴, 1×10¹⁵ CFU of Yersinia/Bacillus. According to some embodiments, Yersinia/Bacillus is applied in an amount sufficient to ensure that an average of about/at least 1×10³, 1×10⁴, 1×10⁵, 1×10⁶, 1×10⁷, 1×10⁸, 1×10⁹, 1×10¹⁰, 1×10¹¹, 1×10¹², 1×10¹³, 1×10¹⁴, 1×10¹⁵ CFU of Yersinia/Bacillus is applied to each acre of treated crops.

In some embodiments, Yersinia/Bacillus is applied at a rate of about 1×10¹ to about 1×10²⁰ CFU per acre of plant growth media. According to some embodiments, Yersinia/Bacillus is applied in an amount sufficient to ensure each acre of plant growth media is treated with about/at least 1×10⁴, 1×10⁵, 1×10⁶, 1×10⁷, 1×10⁸, 1×10⁹, 1×10¹⁰, 1×10¹¹, 1×10¹², 1×10¹³, 1×10¹⁴, 1×10¹⁵ CFU of Yersinia/Bacillus. According to some embodiments, Yersinia/Bacillus is applied in an amount sufficient to ensure that an average of about/at least 1×10³, 1×10⁴, 1×10⁵, 1×10⁶, 1×10⁷, 1×10⁸, 1×10⁹, 1×10¹⁰, 1×10¹¹, 1×10¹², 1×10¹³, 1×10¹⁴, 1×10¹⁵ CFU of Yersinia/Bacillus is applied to each acre of plant growth media.

In some embodiments, compositions of the present disclosure are applied at a rate of about 0.05 to about 100 milliliters and/or grams of composition per kilogram of plant propagation material. According to some embodiments, one or more compositions of the present disclosure is/are applied in an amount sufficient to ensure the plant propagation materials are coated with about/at least 0.05, 0.1, 0.125, 0.15, 0.175, 0.2, 0.225, 0.2.5, 0.275, 0.3, 0.325, 0.35, 0.375, 0.4, 0.425, 0.45, 0.475, 0.5, 0.55, 0.6, 0.65, 0.7, 0.75, 0.8, 0.85, 0.9, 0.95, 1, 1.25, 1.5, 1.75, 2, 2.25, 2.5, 2.75, 3, 3.25, 3.5, 3.75, 4, 4.25, 4.5, 4.75, 5, 5.5, 6, 6.5, 7, 7.5, 8, 8.5, 9, 9.5, 10, 20, 30, 40, 50, 60, 70, 80, 90 or 100 milliliters and/or grams of compositions per kilogram of plant propagation material. According to some embodiments, one or more compositions of the present disclosure is/are applied in an amount sufficient to ensure that an average of about/at least 0.05, 0.1, 0.125, 0.15, 0.175, 0.2, 0.225, 0.2.5, 0.275, 0.3, 0.325, 0.35, 0.375, 0.4, 0.425, 0.45, 0.475, 0.5, 0.55, 0.6, 0.65, 0.7, 0.75, 0.8, 0.85, 0.9, 0.95, 1, 1.25, 1.5, 1.75, 2, 2.25, 2.5, 2.75, 3, 3.25, 3.5, 3.75, 4, 4.25, 4.5, 4.75 or 5 milliliters and/or grams of composition is applied to each seed.

In some embodiments, compositions of the present disclosure are applied at a rate of about 0.5 to about 100 milliliters and/or grams of composition per plant. According to some embodiments, one or more compositions of the present disclosure is/are applied in an amount sufficient to ensure each plant is treated with about/at least 0.05, 0.1, 0.125, 0.15, 0.175, 0.2, 0.225, 0.2.5, 0.275, 0.3, 0.325, 0.35, 0.375, 0.4, 0.425, 0.45, 0.475, 0.5, 0.55, 0.6, 0.65, 0.7, 0.75, 0.8, 0.85, 0.9, 0.95, 1, 1.25, 1.5, 1.75, 2, 2.25, 2.5, 2.75, 3, 3.25, 3.5, 3.75, 4, 4.25, 4.5, 4.75, 5, 5.5, 6, 6.5, 7, 7.5, 8, 8.5, 9, 9.5, 10, 20, 30, 40, 50, 60, 70, 80, 90 or 100 milliliters and/or grams of composition. According to some embodiments, one or more compositions of the present disclosure is/are applied in an amount sufficient to ensure that an average of about/at least 0.05, 0.1, 0.125, 0.15, 0.175, 0.2, 0.225, 0.2.5, 0.275, 0.3, 0.325, 0.35, 0.375, 0.4, 0.425, 0.45, 0.475, 0.5, 0.55, 0.6, 0.65, 0.7, 0.75, 0.8, 0.85, 0.9, 0.95, 1, 1.25, 1.5, 1.75, 2, 2.25, 2.5, 2.75, 3, 3.25, 3.5, 3.75, 4, 4.25, 4.5, 4.75 or 5 milliliters and/or grams of composition is applied to each plant.

In some embodiments, compositions of the present disclosure are applied at a rate of about 0.5 to about 100 milliliters and/or grams of composition per acre of treated crops. According to some embodiments, one or more compositions of the present disclosure is/are applied in an amount sufficient to ensure each acre of treated crops is treated with about/at least 0.05, 0.1, 0.125, 0.15, 0.175, 0.2, 0.225, 0.2.5, 0.275, 0.3, 0.325, 0.35, 0.375, 0.4, 0.425, 0.45, 0.475, 0.5, 0.55, 0.6, 0.65, 0.7, 0.75, 0.8, 0.85, 0.9, 0.95, 1, 1.25, 1.5, 1.75, 2, 2.25, 2.5, 2.75, 3, 3.25, 3.5, 3.75, 4, 4.25, 4.5, 4.75, 5, 5.5, 6, 6.5, 7, 7.5, 8, 8.5, 9, 9.5, 10, 20, 30, 40, 50, 60, 70, 80, 90 or 100 milliliters and/or grams of composition. According to some embodiments, one or more compositions of the present disclosure is/are applied in an amount sufficient to ensure that an average of about/at least 0.05, 0.1, 0.125, 0.15, 0.175, 0.2, 0.225, 0.2.5, 0.275, 0.3, 0.325, 0.35, 0.375, 0.4, 0.425, 0.45, 0.475, 0.5, 0.55, 0.6, 0.65, 0.7, 0.75, 0.8, 0.85, 0.9, 0.95, 1, 1.25, 1.5, 1.75, 2, 2.25, 2.5, 2.75, 3, 3.25, 3.5, 3.75, 4, 4.25, 4.5, 4.75 or 5 milliliters and/or grams of composition is applied to each acre of treated crops.

In some embodiments, compositions of the present disclosure are applied at a rate of about 0.5 to about 100 milliliters and/or grams of composition per acre of plant growth media. According to some embodiments, one or more compositions of the present disclosure is/are applied in an amount sufficient to ensure each acre of plant growth media is treated with about/at least 0.05, 0.1, 0.125, 0.15, 0.175, 0.2, 0.225, 0.2.5, 0.275, 0.3, 0.325, 0.35, 0.375, 0.4, 0.425, 0.45, 0.475, 0.5, 0.55, 0.6, 0.65, 0.7, 0.75, 0.8, 0.85, 0.9, 0.95, 1, 1.25, 1.5, 1.75, 2, 2.25, 2.5, 2.75, 3, 3.25, 3.5, 3.75, 4, 4.25, 4.5, 4.75, 5, 5.5, 6, 6.5, 7, 7.5, 8, 8.5, 9, 9.5, 10, 20, 30, 40, 50, 60, 70, 80, 90 or 100 milliliters and/or grams of composition. According to some embodiments, one or more compositions of the present disclosure is/are applied in an amount sufficient to ensure that an average of about/at least 0.05, 0.1, 0.125, 0.15, 0.175, 0.2, 0.225, 0.2.5, 0.275, 0.3, 0.325, 0.35, 0.375, 0.4, 0.425, 0.45, 0.475, 0.5, 0.55, 0.6, 0.65, 0.7, 0.75, 0.8, 0.85, 0.9, 0.95, 1, 1.25, 1.5, 1.75, 2, 2.25, 2.5, 2.75, 3, 3.25, 3.5, 3.75, 4, 4.25, 4.5, 4.75 or 5 milliliters and/or grams of composition is applied to each acre of plant growth media.

In some embodiments, compositions of the present disclosure are applied in an amount sufficient to ensure the plant propagation materials are coated with about/at least 1×10⁴, 1×10⁵, 1×10⁶, 1×10⁷, 1×10⁸, 1×10⁹, 1×10¹⁰, 1×10¹¹, 1×10¹², 1×10¹³, 1×10¹⁴, 1×10¹⁵ CFU of Yersinia/Bacillus per kilogram of plant propagation material. According to some embodiments, one or more compositions of the present disclosure is/are applied in an amount sufficient to ensure that an average of about/at least 1×10³, 1×10⁴, 1×10⁵, 1×10⁶, 1×10⁷, 1×10⁸, 1×10⁹, 1×10¹⁰, 1×10¹¹, 1×10¹², 1×10¹³, 1×10¹⁴, 1×10¹⁵ CFU of Yersinia/Bacillus is applied to each seed.

In some embodiments, compositions of the present disclosure are applied in an amount sufficient to ensure each plant is treated with about/at least 1×10⁴, 1×10⁵, 1×10⁶, 1×10⁷, 1×10⁸, 1×10⁹, 1×10¹⁰, 1×10¹¹, 1×10¹², 1×10¹³, 1×10¹⁴, 1×10¹⁵ CFU of Yersinia/Bacillus. According to some embodiments, one or more compositions of the present disclosure is/are applied in an amount sufficient to ensure that an average of about/at least 1×10³, 1×10⁴, 1×10⁵, 1×10⁶, 1×10⁷, 1×10⁸, 1×10⁹, 1×10¹⁰, 1×10¹¹, 1×10¹², 1×10¹³, 1×10¹⁴, 1×10¹⁵ CFU of Yersinia/Bacillus is applied to each plant.

In some embodiments, compositions of the present disclosure are applied in an amount sufficient to ensure each acre of treated crops is treated with about/at least 1×10⁴, 1×10⁵, 1×10⁶, 1×10⁷, 1×10⁸, 1×10⁹, 1×10¹⁰, 1×10¹¹, 1×10¹², 1×10¹³, 1×10¹⁴, 1×10¹⁵ CFU of Yersinia/Bacillus. According to some embodiments, one or more compositions of the present disclosure is/are applied in an amount sufficient to ensure that an average of about/at least 1×10³, 1×10⁴, 1×10⁵, 1×10⁶, 1×10⁷, 1×10⁸, 1×10⁹, 1×10¹⁰, 1×10¹¹, 1×10¹², 1×10¹³, 1×10¹⁴, 1×10¹⁵ CFU of Yersinia/Bacillus is applied to each acre of treated crops.

In some embodiments, compositions of the present disclosure are applied in an amount sufficient to ensure each acre of plant growth media is treated with about/at least 1×10⁴, 1×10⁵, 1×10⁶, 1×10⁷, 1×10⁸, 1×10⁹, 1×10¹⁰, 1×10¹¹, 1×10¹², 1×10¹³, 1×10¹⁴, 1×10¹⁵ CFU of Yersinia/Bacillus. According to some embodiments, one or more compositions of the present disclosure is/are applied in an amount sufficient to ensure that an average of about/at least 1×10³, 1×10⁴, 1×10⁵, 1×10⁶, 1×10⁷, 1×10⁸, 1×10⁹, 1×10¹⁰, 1×10¹¹, 1×10¹², 1×10¹³, 1×10¹⁴, 1×10¹⁵ CFU of Yersinia/Bacillus is applied to each acre of plant growth media.

The compositions of the present disclosure may be applied to plants, plant parts and/or plant growth media at any time, including, but not limited to, prior to planting, at the time of planting, after planting, prior to germination, at the time of germination, after germination, prior to seedling emergence, at the time of seedling emergence, after seedling emergence, prior to the vegetative stage, during the vegetative stage, after the vegetative stage, prior to the reproductive stage, during the reproductive stage, after the reproductive stage, prior to flowering, at the time of flowering, after flowering, prior to fruiting, at the time of fruiting, after fruiting, prior to ripening, at the time of ripening, and after ripening. In some embodiments, the compositions are applied to plant propagation materials (e.g., seeds) about/at least 1, 2, 3, 4, 5, 6, 7, 8, 9, 10, 11, 12, 13, 14, 15, 16, 17, 18, 19, 20, 21, 22, 23, 24, 28, 32, 36, 40, 44, 48, 52, 56, 60, 64, 68, 72, 76, 80, 84, 88, 92, 96, 100, 104 weeks prior to planting.

In some embodiments, the compositions are applied to plant propagation materials (e.g., seeds) at the time of planting In some embodiments, the compositions are applied to plant propagation materials (e.g., seeds) after planting but before germination In some embodiments, the compositions are applied to plants following emergence.

The present disclosure extends to plants and plant parts (e.g., coated plant propagation materials) that have been treated with the compositions, to plants that grow from plant parts (e.g., coated plant propagation materials) that have been treated with the compositions, to plant parts harvested from plants that have been treated with the compositions, to plant parts harvested from plants that grow from plant parts (e.g., coated plant propagation materials) that have been treated with the compositions, to processed products derived from plants that have been treated with with the compositions, to processed products derived from plants that grow from plant parts (e.g., coated plant propagation materials) that have been treated with the compositions, to crops comprising a plurality of plants that have been treated with the compositions, and to crops comprising a plurality of plants that grow from plant parts (e.g., coated plant propagation materials) that have been treated with the compositions.

In some embodiments, the present disclosure provides coated plant propagation materials comprising, consisting essentially of, or consisting of a plant propagation material and a coating that covers at least a portion of the outer surface of the plant propagation material, said coating comprising, consisting essentially of, or consisting the compositions of the present disclosure.

In some embodiments, the coating comprises two, three, four, five or more layers. According to some embodiments, the coating comprises an inner layer that contains Yersinia/Bacillus and one or more outer layers free or substantially free of microorganisms. In some embodiments, the coating comprises an inner layer that is a composition of the present disclosure and an outer layer that is equivalent to a composition of the present disclosure except that it does not contain Yersinia/Bacillus.

In some embodiments, the coating comprises, consists essentially of, or consists of an composition of the present disclosure and a drying powder. Drying powders may be applied in any suitable amount(s)/concentration(s). The absolute value of the amount/concentration that is/are sufficient to cause the desired effect(s) may be affected by factors such as the type, size and volume of material to which the composition will be applied, the type(s) of microorganisms in the composition, the number of microorganisms in the composition, the stability of the microorganisms in the composition and storage conditions (e.g., temperature, relative humidity, duration). Those skilled in the art will understand how to select an effective amount/concentration using routine dose-response experiments. Guidance for the selection of appropriate amounts/concentrations can be found, for example, in International Patent Application Nos. PCT/US2016/050529 and PCT/US2016/050647 and U.S. Provisional Patent Application Nos. 62/296,798; 62/271,857; 62/347,773; 62/343,217; 62/296,784; 62/271,873; 62/347,785; 62/347,794; and 62/347,805. In some embodiments, the drying powder is applied in an amount ranging from about 0.5 to about 10 grams of drying powder per kilogram of plant propagation material. For example, in some embodiments, about 0.5, 1, 1.25, 1.5, 1.75, 2, 2.25, 2.5, 2.75, 3, 3.25, 3.5, 3.75, 4, 4.25, 4.5, 4.75, 5, 5.5, 6, 6.5, 7, 7.5, 8, 8.5, 9, 9.5, 10 grams or more of drying powder (e.g., drying powder comprising magnesium stearate, magnesium sulfate, powdered milk, silica, soy lecithin and/or talc) is applied per kilogram of seed. In some embodiments, a drying powder comprising calcium stearate, attapulgite clay, montmorillonite clay, graphite, magnesium stearate, silica (e.g., fumed silica, hydrophobically-coated silica and/or precipitated silica) and/or talc is applied to seeds coated with a composition of the present disclosure at a rate of about 1, 1.25, 1.5, 1.75, 2, 2.25, 2.5, 2.75, or 3 grams per kilogram of seed.

In some embodiments, the coating completely covers the outer surface of the plant propagation material.

In some embodiments, the average thickness of the coating is at least 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 4, 4.5, 5 μm or more. In some embodiments, the average thickness of the coating is about 1.5 to about 3.0 μm.

The present disclosure extends to kits comprising, consisting essentially of, or consisting of one or more plants and/or plant parts (e.g., coated plant propagation materials) that have been treated with the compositions of the present disclosure and a container housing the treated plant(s) and/or plant part(s). In some embodiments, the kit further comprises one or more oxygen scavengers, such as activated carbon, ascorbic acid, iron powder, mixtures of ferrous carbonate and metal halide catalysts, sodium chloride and/or sodium hydrogen carbonate.

The container may comprise any suitable material(s), including, but not limited to, materials that reduce the amount of light, moisture and/or oxygen that contact the coated plant propagation material when the container is sealed. In some embodiments, the container comprises, consists essentially of, or consists of a material having light permeability of less than about 1, 2, 3, 4, 5, 6, 7, 8, 9, 10, 15, 20, 25, 30, 35, 40, 45, 50, 55, 60, 65, 70 or 75%. In some embodiments, the container comprises, consists essentially of, or consists of a material having an oxygen transmission rate of less than about 5, 10, 15, 20, 25, 30, 35, 40, 45, 50, 60, 70, 80, 90, 100, 125, 150, 175, 200, 225, 250, 275, 300, 325, 350, 375, 400, 425, 450, 475, or 500 cm³/m² day (as measured in accordance with ASTM D3985).

In some embodiments, the container reduces the amount of ambient light that reaches said coated plant propagation material by about 5, 10, 15, 20, 25, 30, 35, 40, 45, 50, 55, 60, 65, 70, 75, 80, 85, 90, 95 or 100% when sealed.

In some embodiments, the container reduces the amount of ambient moisture that reaches said plant propagation material by about 5, 10, 15, 20, 25, 30, 35, 40, 45, 50, 55, 60, 65, 70, 75, 80, 85, 90, 95 or 100% when sealed.

In some embodiments, the container reduces the amount of ambient oxygen that reaches said plant propagation material by about 5, 10, 15, 20, 25, 30, 35, 40, 45, 50, 55, 60, 65, 70, 75, 80, 85, 90, 95 or 100% when sealed.

In some embodiments, kits of the present disclosure comprise 1, 2, 3, 4, 5 or more additional containers. The additional containers may comprise any suitable component(s) or composition(s), including, but not limited to, agriculturally beneficial microorganisms, biostimulants, drying agents, nutrients, oxidation control components and pesticides. Examples of agriculturally beneficial microorganisms, biostimulants, drying agents, nutrients, oxidation control components and pesticides that may be included in the additional containers are described above.

The present disclosure extends to animal feed compositions comprising, consisting essentially of or consisting of a food component and a microbial component, said microbial component comprising, consisting essentially of, or consisting of the compositions of the present disclosure.

Animal feed compositions of the present disclosure may comprise any suitable food component, including, but not limited to, fodder (e.g., grains, hay, legumes, silage and/or straw) and forage (e.g., grass).

Animal feed compositions of the present disclosure may be fed to any suitable animal, including, but not limited to, farm animals, zoo animals, laboratory animals and/or companion animals In some embodiments, the animal feed composition is formulated to meet the dietary needs of birds (e.g., chickens, ducks, quails and/or turkeys), bovids (e.g., antelopes, bison, cattle, gazelles, goats, impala, oxen, sheep and/or wildebeests), canines, cervids (e.g., caribou, deer, elk and/or moose), equines (e.g., donkeys, horses and/or zebras), felines, fish, pigs, rabbits, rodents (e.g., guinea pigs, hamsters, mice and/or rats) and the like.

The present disclosure extends to methods and uses for the compositions of the present disclosure.

In some embodiments, methods and uses of the present disclosure comprise, consist essentially of or consist of applying the compositions disclosed herein to a plant or plant part (e.g., plant propagation material). As noted above, the compositions of the present disclosure may be applied to any type of plant, to any part/portion of a plant, in any suitable manner, in any suitable amount(s)/concentration(s) and at any suitable time(s). According to some embodiments, methods and uses of the present disclosure comprise, consist essentially of or consist of applying the compositions to a monocotyledonous plant or plant part (e.g., a cereal or pseudocereal plant or plant part, optionally, barley, buckwheat, corn, millet, oats, quinoa, rice, lye, sorghum or wheat).

In some embodiments, methods and uses of the present disclosure comprise, consist essentially of or consist of applying the disclosed compositions to a plant growth medium. As noted above, the compositions of the present disclosure may be applied to any plant growth medium, in any suitable manner, in any suitable amount(s)/concentration(s) and at any suitable time(s).

In some embodiments, methods and uses of the present disclosure comprise, consist essentially of or consist of introducing a plant or plant part (e.g., plant propagation material) that has been treated with the disclosed compositions into a plant growth medium (e.g., a soil). Such methods may further comprise introducing one or more nutrients (e.g., nitrogen and/or phosphorous) into the plant growth medium. Any suitable nutrient(s) may be added to the growth medium, including, but not limited to, rock phosphate, monoammonium phosphate, diammonium phosphate, monocalcium phosphate, super phosphate, triple super phosphate, ammonium polyphosphate, fertilizers comprising one or more phosphorus sources, and combinations thereof.

In some embodiments, methods and uses of the present disclosure comprise, consist essentially of or consist of growing a plant from a plant propagation material that has been treated with the compositions of the present disclosure.

The compositions may be used to kill pests (e.g., insects), retard their grown, or prevent pests from infecting, infesting, killing/destroying or retarding growth of a plant. In some embodiments, the compositions may enhance plant growth. In some embodiments, the compositions disclosed herein are combinations of one or more Yersinia/Bacillus microbes and one or more substances, like a pesticide or an insecticide. In some embodiments, one or more of the effects of the compositions, on plants or insects for example, are less than additive (e.g., antagonistic) as compared to the effects of the individual components of the composition, or groups of individual of the components that are less than all of the components of the composition. In some embodiments, one or more of the effects of the compositions, on plants or insects for example, are additive as compared to the effects of the individual components of the composition, or groups of individual of the components that are less than all of the components of the composition. In some embodiments, one or more of the effects of the compositions, on plants or insects for example, give unexpected results as compared to the effects of the individual components of the composition, or groups of individual of the components that are less than all of the components of the composition.

In some embodiments, unexpected results of the compositions as compared to individual components or groups of individual components of a composition may be described by a performance index. In some embodiments, the performance index may be the effect of the combination divided by the sum of effects of the individual components of the composition.

The compositions may be used to enhance growth and/or yield of plants. In some embodiments, application of the compositions enhances 1, 2, 3, 4, 5 or more growth characteristics and/or 1, 2, 3, 4, 5 or more yield characteristics by about/at least 5, 10, 15, 20, 25, 30, 35, 40, 45, 50, 55, 60, 65, 70, 75, 80, 85, 90, 95, 100, 105, 110, 115, 120, 125, 150, 175, 200, 225, 250% or more as compared to one or more controls (e.g., untreated control plants and/or plants treated with an alternative microbial strain). For example, in some embodiments, application of the compositions enhances plant yield by about/at least 0.25, 0.5, 0.75, 1, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 4, 4.1, 4.2, 4.3, 4.4, 4.5, 4.6, 4.7, 4.8, 4.9, or 5 bushels per acre as compared to the yield of untreated control plants and/or plants treated with an alternative microbial strain.

The compositions may likewise be used to enhance plant growth and/or yield. In some embodiments, application of an composition of the present disclosure enhances 1, 2, 3, 4, 5 or more plant growth characteristics and/or 1, 2, 3, 4, 5 or more plant yield characteristics by about/at least 5, 10, 15, 20, 25, 30, 35, 40, 45, 50, 55, 60, 65, 70, 75, 80, 85, 90, 95, 100, 105, 110, 115, 120, 125, 150, 175, 200, 225, 250% or more as compared to a control composition (e.g., a control composition that is identical to the composition of the present disclosure except that it lacks the disclosed compositions). For example, in some embodiments, application of a composition of the present disclosure enhances plant yield by about/at least 0.25, 0.5, 0.75, 1, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 4, 4.1, 4.2, 4.3, 4.4, 4.5, 4.6, 4.7, 4.8, 4.9, or 5 bushels per acre as compared to a control composition.

Accordingly, in some embodiments, methods and uses of the present disclosure comprise, consist essentially of or consist of applying the disclosed compositions to seeds, to the plant growth medium in which said seeds are being or will be grown, and/or to the plant(s) that grow(s) from said seeds.

In some embodiments, the compositions are applied to seeds in an amount/concentration effective to enhance 1, 2, 3, 4, 5 or more plant growth characteristics (e.g., biomass) and/or 1, 2, 3, 4, 5 or more plant yield characteristics (e.g., bushels per acre) of the plant that grows from said seed by at least about 5, 10, 15, 20, 25, 30, 35, 40, 45, 50, 55, 60, 65, 70, 75, 80, 85, 90, 95, 100, 105, 110, 115, 120, 125, 150, 175, 200, 225, 250% or more as compared to one or more control plants (e.g., plants grown from untreated seeds and/or plants grown from seeds treated with a control). According to some embodiments, the disclosed compositions are applied to seeds in an amount effective to enhance yield by about/at least 0.25, 0.5, 0.75, 1, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 4, 4.1, 4.2, 4.3, 4.4, 4.5, 4.6, 4.7, 4.8, 4.9, or 5 bushels per acre.

In some embodiments, the compositions are introduced into a plant growth medium (e.g., soil) in an amount/concentration effective to enhance 1, 2, 3, 4, 5 or more plant growth characteristics (e.g., biomass) and/or 1, 2, 3, 4, 5 or more plant yield characteristics (e.g., bushels per acre) of plants grown therein by at least about 5, 10, 15, 20, 25, 30, 35, 40, 45, 50, 55, 60, 65, 70, 75, 80, 85, 90, 95, 100, 105, 110, 115, 120, 125, 150, 175, 200, 225, 250% or more as compared to one or more controls (e.g., plants grown in untreated soil and/or plants grown in soil treated with an alternative microbial strain). According to some embodiments, the compositions are introduced into the plant growth medium in an amount effective to enhance plant yield by about/at least 0.25, 0.5, 0.75, 1, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 4, 4.1, 4.2, 4.3, 4.4, 4.5, 4.6, 4.7, 4.8, 4.9, or 5 bushels per acre.

### Deposit of Biological Materials

Y. entomophaga NRRL B-67598, Y. entomophaga NRRL B-67599, Y. entomophaga NRRL B-67600 and Y. entomophaga NRRL B-67601 were deposited on Mar. 15, 2018, under the terms of the Budapest Treaty on the International Recognition of the Deposit of Microorganisms for the Purposes of Patent Procedure at the Agricultural Research Service Culture Collection, 1815 North University Street, Peoria, Ill. 61604, U.S.A.

Y. entomophaga NRRL B-67598, Y. entomophaga NRRL B-67599, Y. entomophaga NRRL B-67600 and Y. entomophaga NRRL B-67601 were deposited under conditions that assure access to the culture will be available during the pendency of this patent application to one determined by the Commissioner of Patents and Trademarks to be entitled thereto under 37 C.F.R. § 1.14 and 35 U.S.C. § 122. Each deposit represents a pure culture of the deposited strain. Each deposit is available as required by foreign patent laws in countries wherein counterparts of the subject application or its progeny are filed. However, it should be understood that the availability of a deposit does not constitute a license to practice the subject invention in derogation of patent rights granted by governmental action.

### EXAMPLES

The following examples are not intended to be a detailed catalogue of all the different ways in which the present disclosure may be implemented or of all the features that may be added to the present disclosure. Subjects skilled in the art will appreciate that numerous variations and additions to the various embodiments may be made without departing from the present disclosure. Hence, the following descriptions are intended to illustrate some particular embodiments of the invention and not to exhaustively specify all permutations, combinations and variations thereof.

### Example 1

### Growth of Yersinina Entomophaga and Bacillus Thuringiensis, and Determination of Dose

Yersinina entomophaga strains were grown in LB broth at room temperature with 200 rpm shaking for 20 hours. Amounts of Yersinina entomophaga used in the assays described below were determined using CFU assays.

Bacillus thuringiensis var tenebrionis (Btt) (IBL1410) or Bacillus thuringiensis subspecies kurstaki, ABTS-351 (Btk) was grown in LB broth at 35 degrees C. with 200 rpm using 50 ml of media in a 250 ml baffled flask, for 20 hours. At 20 hours, the majority of cells were vegetative cells, based on observation using a phase-contrast microscope. In some experiments, amounts of Bacillus thuringiensis are expressed as a dilution of the DiPel® product (% w/w). DiPel® contains Bacillus thuringiensis subspecies kurstaki, ABTS-351 strain (Valent BioSciences).

The insects used in the assays that follow in the Examples below, were generally screened with different amounts of Yersinina entomophaga or Bacillus thuringiensis cells, to determine amounts of the cells that provided activity in the linear range of a dose-response killing curve for that insect. In the experiments used to obtain the data shown below, amounts of Yersinina entomophaga and Bacillus thuringiensis in the linear range of the dose-response curves were used. These amounts of these bacteria allowed for detection of possible interactions between the organisms in the insect killing studies, when the organisms were used together in the assays.

### Example 2

### Yersinia Entomophaga Strain O43NEW with Bacillus Thuringiensis Subspecies Kurstaki Against Black Cutworm

Cabbage leaf disks were dipped in either Yersinia entomophaga (Ye) strain O43NEW at a concentration of 1×10⁵ CFU/mL in phosphate buffer, Bacillus thuringiensis subspecies kurstaki (Btk) as DiPel® at 0.12% w/w in phosphate buffer, or a 1:1 combination of these two actives. Controls were dipped in phosphate buffer. After the cabbage disks had dried for 1 hr, a single 3^{rd} instar black cutworm larva was added to each individual cabbage disk. A total of 20 insects were evaluated for each treatment.

TABLE 1
Mortality of 3^{rd} instar black cutworm treated with B. thuringiensis
subspecies kurstaki (Btk), Y. entomophaga (Ye) strain
O43NEW, and a combination of both

| | LT50 (95% confidence | % Mortality | % Mortality |
|---|---|---|---|
| Treatment | intervals) in days | (2DAT) | (5DAT) |
| Btk (DiPel ®) at | 3.84 (3.36-4.37) | 39 | 66 |
| 0.12% w/w | | | |
| Ye O43NEW at | 6.95 (6.17-7.82) | 6 | 22 |
| 1 × 10⁵ CFU/mL | | | |
| Btk + Ye O43NEW | 2.10 (1.78-2.47) | 65 | 95 |

LT50 is the estimated time to kill 50% of the insects based on Probit analysis, % mortality is Abbott's corrected and DAT is the days after treatment.

The combination of Yersinia entomophaga strain O43NEW with Bacillus thuringiensis subspecies kurstaki resulted in a significantly lower LT50 than either active alone. The data in the above table also show the percent mortality of black cutworm, at 2 and 5 days after treatment, for Ye O43NEW alone, for Btk alone, and for combinations of Ye O43NEW and Btk.

### Example 3

### Yersinia Entomophaga Strains with Bacillus Thuringiensis Subspecies Kurstaki Against Cabbage Looper

Cabbage leaf disks were dipped in either Yersinia entomophaga (Ye) strain O43NEW at a concentration of 1×10³ CFU/mL in phosphate buffer, Bacillus thuringiensis subspecies kurstaki (Btk) as DiPel® at 0.0012% w/w in phosphate buffer, or a 1:1 combination of these two actives. Controls were dipped in phosphate buffer. After the cabbage disks had dried for 1 hr, a single 3^{rd} instar cabbage looper larva was added to each individual cabbage disk. A total of 20 insects were evaluated for each treatment.

TABLE 2
Mortality of 3^{rd} instar cabbage looper treated with
B. thuringiensis subspecies kurstaki (Btk), Y. entomophaga
(Ye) strain O43NEW, and a combination of both

| | LT50 (95% confidence |
|---|---|
| Treatment | intervals) in days |
| Btk (DiPel ®) at 0.0012% w/w | 8.71 (7.53-10.10) |
| Ye at 1 × 10³ CFU/mL | 8.43 (7.29-9.77) |
| Btk + Ye | 5.54 (4.71-6.46) |

LT50 is the estimated time to kill 50% of the insects based on Probit analysis.

The combination of Yersinia entomophaga strain O43NEW with Bacillus thuringiensis subspecies kurstaki resulted in a significantly lower LT50 than either active alone.

Another experiment was performed similarly, except that the amounts of Yersinia entomophaga strain O43NEW and Bacillus thuringiensis subspecies kurstaki were different than those used in the previous experiment (see table below). A total of 24 insects were evaluated for each treatment. The data from that experiment are shown in the table below.

TABLE 3
Mortality of 3^{rd} instar cabbage looper treated with
B. thuringiensis subspecies kurstaki (Btk), Y. entomophaga
(Ye) strain O43NEW, and a combination of both

| | LT50 (95% confidence | % Mortality |
|---|---|---|
| Treatment | intervals) in days | (2DAT) |
| Btk (ABTS-351) at 1 × 10⁷ | 3.01 (2.75-3.29) | 25 |
| CFL/mL | | |
| Ye O43NEW at 1 × 10⁷ CFU/mL | 2.48 (2.23-2.76) | 17 |
| Btk + Ye O43NEW | 1.12 (0.97-1.29) | 96 |

LT50 is the estimated time to kill 50% of the insects based on Probit analysis, % mortality is Abbott's corrected and DAT is the days after treatment.

The combination of Yersinia entomophaga strain O43NEW with Bacillus thuringiensis subspecies kurstaki resulted in a significantly lower LT50 than either active alone. The data in the above table also show the percent mortality of cabbage looper, at 2 days after treatment, for Ye O43NEW alone, for Btk alone, and for combinations of Ye and Btk.

Another experiment was performed similarly, except that the Yersinia entomophaga strain used was strain O23ZMJ. A total of 24 insects were evaluated for each treatment. The data from that experiment are shown in the table below.

TABLE 4
Mortality of 3^{rd} instar cabbage looper with B. thuringiensis
subspecies kurstaki (Btk), Y. entomophaga (Ye)
strain O23ZMJ, and a combination of both

| | LT50 (95% confidence | % Mortality |
|---|---|---|
| Treatment | intervals) in days | (2DAT) |
| Btk (ABTS-351) at 1 × 10⁷ | 2.66 (2.39-2.95) | 33 |
| CFU/mL | | |
| Ye O23ZMJ at 1 × 10⁷ CFU/mL | 2.61 (2.23-2.90) | 17 |
| Btk + Ye O23ZMJ | 1.34 (1.15-1.57) | 100.0 |

LT50 is the estimated time to kill 50% of the insects based on Probit analysis, % mortality is Abbott's corrected and DAT is the days after treatment.

The combination of Yersinia entomophaga strain O23ZMJ with Bacillus thuringiensis subspecies kurstaki resulted in a significantly lower LT50 than either active alone. The data in the above table also show the percent mortality of cabbage looper, at 2 days after treatment, for Ye O23ZMJ alone, for Btk alone, and for combinations of Ye O23ZMJ and Btk.

Another experiment was performed similarly, except that the Yersinia entomophaga strain used was strain O24G3R. A total of 24 insects were evaluated for each treatment. The data from that experiment are shown in the table below.

TABLE 5
Mortality of 3^{rd} instar cabbage looper with B. thuringiensis
subspecies kurstaki (Btk), Y. entomophaga (Ye)
strain O24G3R, and a combination of both

| | LT50 (95% confidence | % Mortality |
|---|---|---|
| Treatment | intervals) in days | (2DAT) |
| Btk (ABTS-351) at 1 × 10⁷ | 2.66 (2.39-2.95) | 33 |
| CFU/mL | | |
| Ye O24G3R at 1 × 10⁷ CFU/mL | 2.43 (2.18-2.71) | 15 |
| Btk + Ye O24G3R | 1.41 (1.22-1.62) | 88 |

LT50 is the estimated time to kill 50% of the insects based on Probit analysis, % mortality is Abbott's corrected and DAT is the days after treatment.

The combination of Yersinia entomophaga strain O24G3R with Bacillus thuringiensis subspecies kurstaki resulted in a significantly lower LT50 than either active alone. The data in the above table also show the percent mortality of cabbage looper, at 2 days after treatment, for Ye O24G3R alone, for Btk alone, and for combinations of Ye O24G3R and Btk.

Another experiment was performed similarly, except that the Yersinia entomophaga strain used was strain O348UX. A total of 24 insects were evaluated for each treatment. The data from that experiment are shown in the table below.

TABLE 6
Mortality of 3^{rd} instar cabbage looper with B. thuringiensis
subspecies kurstaki (Btk), Y. entomophaga (Ye)
strain O348UX, and a combination of both

| | LT50 (95% confidence | % Mortality |
|---|---|---|
| Treatment | intervals) in days | (2DAT) |
| Btk (ABTS-351) at 1 × 10⁷ | 2.66 (2.39-2.95) | 33 |
| CFU/mL | | |
| Ye O348UX at 1 × 10⁷ CFU/mL | 2.56 (2.29-2.87) | 8 |
| Btk + Ye O348UX | 1.43 (1.23-1.66) | 96 |

LT50 is the estimated time to kill 50% of the insects based on Probit analysis, % mortality is Abbott's corrected and DAT is the days after treatment.

The combination of Yersinia entomophaga strain O348UX with Bacillus thuringiensis subspecies kurstaki resulted in a significantly lower LT50 than either active alone. The data in the above table also show the percent mortality of cabbage looper, at 2 days after treatment, for Ye O348UX alone, for Btk alone, and for combinations of Ye O348UX and Btk.

Another experiment was performed similarly, except that the Yersinia entomophaga strain used was strain O33ZDX. A total of 24 insects were evaluated for each treatment. The data from that experiment are shown in the table below.

TABLE 7
Mortality of 3^{rd} instar cabbage looper with B. thuringiensis
subspecies kurstaki (Btk), Y. entomophaga (Ye)
strain O33ZDX, and a combination of both

| | LT50 (95% confidence | % Mortality |
|---|---|---|
| Treatment | intervals) in days | (2DAT) |
| Btk (ABTS-351) at 1 × 10⁷ | 2.66 (2.39-2.95) | 33 |
| CFU/mL | | |
| Ye O33ZDX at 1 × 10⁷ CFU/mL | 2.40 (2.13-2.70) | 8 |
| Btk + Ye O33ZDX | 1.77 (1.57-2.00) | 79 |

LT50 is the estimated time to kill 50% of the insects based on Probit analysis, % mortality is Abbott's corrected and DAT is the days after treatment.

The combination of Yersinia entomophaga strain O33ZDX with Bacillus thuringiensis subspecies kurstaki resulted in a significantly lower LT50 than either active alone. The data in the above table also show the percent mortality of cabbage looper, at 2 days after treatment, for Ye O33ZDX alone, for Btk alone, and for combinations of Ye O33ZDX and Btk.

### Example 4

### Yersinia Entomophaga Strain O43NEW with Bacillus Thuringiensis Subspecies Kurstaki Against Fall Armyworm

Cabbage leaf disks were dipped in either Yersinia entomophaga strain O43NEW (Ye) at a concentration of 1×10⁷ CFU/mL in phosphate buffer, Bacillus thuringiensis subspecies kurstaki (Btk) as DiPel® at 0.12% w/w in phosphate buffer, or a 1:1 combination of these two actives. Controls were dipped in phosphate buffer. After the cabbage disks had dried for 1 hr, a single 3^{rd} instar fall armyworm larva was added to each individual cabbage disk. A total of 20 insects were evaluated for each treatment.

TABLE 8
Mortality of 3^{rd} instar fall armyworm treated with B. thuringiensis subspecies
kurstaki (Btk), Y. entomophaga (Ye) strain O43NEW, and a combination of both

| | LT50 (95% confidence | % Mortality | % Mortality | % Mortality |
|---|---|---|---|---|
| Treatment | intervals) in days | (2DAT) | (5DAT) | (6DAT) |
| Btk (DiPel ®) at | 6.77 (6.05-7.57) | 0 | 25 | 30 |
| 0.12% w/w | | | | |
| Ye O43NEW at 1 × 10⁷ | 7.07 (6.45-7.74) | 0 | 13 | 37 |
| CFU/mL | | | | |
| Btk + Ye O43NEW | 4.35 (3.80-4.98) | 5 | 60 | 70 |

LT50 is the estimated time to kill 50% of the insects based on Probit analysis, % mortality is Abbott's corrected and DAT is the days after treatment.

The combination of Yersinia entomophaga strain O43NEW with Bacillus thuringiensis subspecies kurstaki resulted in a significantly lower LT50 than either active alone. The data in the above table also show the percent mortality of fall armyworm, at 2, 5 and 6 days after treatment, for Ye alone, for Btk alone, and for combinations of Ye and Btk.

### Example 5

### Yersinia Entomophaga Strains with Bacillus Thuringiensis Subspecies Kurstaki Against Tobacco Budworm

Cabbage leaf disks were dipped in either Yersinia entomophaga (Ye) strain O43NEW, Bacillus thuringiensis subspecies kurstaki (Btk) as DiPei® 0.0012% w/w in phosphate buffer or a 1:1 combination of these two actives. The concentration of Ye O43NEW for the experiment that produced the data shown in Table 9 was 1×10⁴ CFU/mL in phosphate buffer, and for the experiment that produced the data shown in Table 10 was 1×10⁵ CFU/mL, in phosphate buffer. Controls were dipped in phosphate buffer. After the cabbage disks had dried for 1 hr, a single 4^{th} instar tobacco budworm larva was added to each individual cabbage disk for the experiment that produced the data shown in Table 9, and a single 2^{nd} instar tobacco budworm larva was added to each individual cabbage disk for the experiment that produced the data shown in Table 10. A total of 10 insects were evaluated for each treatment shown in Table 9, and a total of 20 insects were evaluated for each treatment shown in Table 10.

TABLE 9
Mortality of 4^{th} instar tobacco budworm treated with B. thuringiensis subspecies
kurstaki (Btk), Y. entomophaga (Ye) strain O43NEW, and a combination of both

| | LT50 (95% confidence | % Mortality | % Mortality | % Mortality |
|---|---|---|---|---|
| Treatment | intervals) in days | (4DAT) | (5DAT) | (6DAT) |
| Btk (DiPel ®) at | 8.91 (8.00-9.93) | 0 | 20 | 30 |
| 0.0012% w/w | | | | |
| Ye O43NEW at 1 × 10⁴ | 7.83 (7.01-8.73) | 0 | 30 | 40 |
| CFU/mL | | | | |
| Btk + Ye O43NEW | 4.64 (4.05-5.29) | 10 | 80 | 90 |

LT50 is the estimated time to kill 50% of the insects based on Probit analysis, % mortality is Abbott's corrected and DAT is the days after treatment.

The combination of Yersinia entomophaga strain O43NEW with Bacillus thuringiensis subspecies kurstaki resulted in a significantly lower LT50 than either active alone (Table 9). The data in the above table also show the percent mortality of tobacco budworm at 4, 5 and 6 days after treatment, for Ye O43NEW alone, for Btk alone, and for combinations of Ye O43NEW and Btk.

TABLE 10
Mortality of 2^{nd} instar tobacco budworm treated with
B. thuringiensis subspecies kurstaki (Btk), Y. entomophaga
(Ye) strain O43NEW, and a combination of both

| | LT50 (95% confidence |
|---|---|
| Treatment | intervals) in days |
| Btk (DiPel ®) at 0.0012% w/w | 5.99 (5.48-6.52) |
| Ye O43NEW at 1 × 10⁵ CFU/mL | 4.59 (4.09-5.10) |
| Btk + Ye O43NEW | 3.04 (2.54-3.63) |

LT50 is the estimated time to kill 50% of the insects based on Probit analysis.

The combination of Yersinia entomophaga strain O43NEW with Bacillus thuringiensis subspecies kurstaki resulted in a significantly lower LT50 than either active alone (Table 10).

Another experiment was performed similarly, except that the amounts of Yersinia entomophaga strain O43NEW and Bacillus thuringiensis subspecies kurstaki were different than those used in the previous experiment (see table below). A total of 20 insects were evaluated for each treatment. The data from that experiment are shown in the table below.

TABLE 11
Mortality of 2^{nd} instar tobacco budworm with B. thuringiensis
subspecies kurstaki (Btk), Y. entomophaga (Ye)
strain O43NEW, and a combination of both

| | LT50 (95% confidence | % Mortality |
|---|---|---|
| Treatment | intervals) in days | (2DAT) |
| Btk (ABTS-351) at 1 × 10⁷ | 2.23 (1.95-2.53) | 21 |
| CFU/mL | | |
| Ye O43NEW at 1 × 10⁷ CFU/mL | 2.24 (1.96-2.54) | 25 |
| Btk + Ye O43NEW | 1.32 (1.12-1.56) | 96 |

LT50 is the estimated time to kill 50% of the insects based on Probit analysis, % mortality is Abbott's corrected and DAT is the days after treatment.

The combination of Yersinia entomophaga strain O43NEW with Bacillus thuringiensis subspecies kurstaki resulted in a significantly lower LT50 than either active alone. The data in the above table also show the percent mortality of tobacco budworm at 2 days after treatment, for Ye O43NEW alone, for Btk alone, and for combinations of Ye O43NEW and Btk.

Another experiment was performed similarly, except that a different strain of Yersinia entomophaga was used (strain O23ZMJ). A total of 24 insects were evaluated for each treatment. The data from that experiment are shown in the table below.

TABLE 12
Mortality of 2^{nd} instar tobacco budworm with B. thuringiensis
subspecies kurstaki (Btk), Y. entomophaga (Ye)
strain O23ZMJ, and a combination of both

| | LT50 (95% confidence | % Mortality |
|---|---|---|
| Treatment | intervals) in days | (2DAT) |
| Btk at 1 × 10⁷ CFU/mL | 2.60 (2.32-2.91) | 9 |
| Ye O23ZMJ at 1 × 10⁷ CFU/mL | 2.64 (2.37-2.93) | 4 |
| Btk + Ye O23ZMJ | 1.40 (1.21-1.61) | 96 |

LT50 is the estimated time to kill 50% of the insects based on Probit analysis, % mortality is Abbott's corrected and DAT is the days after treatment.

The combination of Yersinia entomophaga strain O23ZMJ with Bacillus thuringiensis subspecies kurstaki resulted in a significantly lower LT50 than either active alone. The data in the above table also show the percent mortality of tobacco budworm at 2 days after treatment, for Ye O23ZMJ alone, for Btk alone, and for combinations of Ye O23ZMJ and Btk.

Another experiment was performed similarly, except that a different strain of Yersinia entomophaga was used (strain O24G3R). A total of 24 insects were evaluated for each treatment. The data from that experiment are shown in the table below.

TABLE 13
Mortality of 2^{nd} instar tobacco budworm with B. thuringiensis
subspecies kurstaki (Btk), Y. entomophaga (Ye)
strain O24G3R, and a combination of both

| | LT50 (95% confidence | % Mortality |
|---|---|---|
| Treatment | intervals) in days | (2DAT) |
| Btk (ABTS-351) at 1 × 10⁷ | 2.60 (2.32-2.91) | 9 |
| CFU/mL | | |
| Ye O24G3R at 1 × 10⁷ CFU/mL | 3.24 (2.96-3.54) | 21 |
| Btk + Ye O24G3R | 1.36 (1.19-1.56) | 83 |

LT50 is the estimated time to kill 50% of the insects based on Probit analysis, % mortality is Abbott's corrected and DAT is the days after treatment.

The combination of Yersinia entomophaga strain O24G3R with Bacillus thuringiensis subspecies kurstaki resulted in a significantly lower LT50 than either active alone. The data in the above table also show the percent mortality of tobacco budworm at 2 days after treatment, for Ye O24G3R alone, for Btk alone, and for combinations of Ye O24G3R and Btk.

Another experiment was performed similarly, except that a different strain of Yersinia entomophaga was used (strain O348UX). A total of 24 insects were evaluated for each treatment. The data from that experiment are shown in the table below.

TABLE 14
Mortality of 2^{nd} instar tobacco budworm with B. thuringiensis
subspecies kurstaki (Btk), Y. entomophaga (Ye)
strain O348UX, and a combination of both

| | LT50 (95% confidence | % Mortality |
|---|---|---|
| Treatment | intervals) in days | (2DAT) |
| Btk (ABTS-351) at 1 × 10⁷ | 2.60 (2.32-2.91) | 9 |
| CFU/mL | | |
| Ye O348UX at 1 × 10⁷ CFU/mL | 3.02 (2.74-3.32) | 8 |
| Btk + Ye O348UX | 1.24 (1.06-1.43) | 100.0 |

LT50 is the estimated time to kill 50% of the insects based on Probit analysis, % mortality is Abbott's corrected and DAT is the days after treatment.

The combination of Yersinia entomophaga strain O348UX with Bacillus thuringiensis subspecies kurstaki resulted in a significantly lower LT50 than either active alone. The data in the above table also show the percent mortality of tobacco budworm at 2 days after treatment, for Ye O348UX alone, for Btk alone, and for combinations of Ye O348UX and Btk.

Another experiment was performed similarly, except that a different strain of Yersinia entomophaga was used (strain O33ZDX). A total of 24 insects were evaluated for each treatment. The data from that experiment are shown in the table below.

TABLE 15
Mortality of 2^{nd} instar tobacco budworm with B. thuringiensis
subspecies kurstaki (Btk), Y. entomophaga (Ye)
strain O33ZDX, and a combination of both

| | LT50 (95% confidence | % Mortality |
|---|---|---|
| Treatment | intervals) in days | (2DAT) |
| Btk (ABTS-351) at 1 × 10⁷ | 2.60 (2.32-2.91) | 9 |
| CFU/mL | | |
| Ye O33ZDX at 1 × 10⁷ CFU/mL | 2.70 (2.44-2.98) | 25 |
| Btk + Ye O33ZDX | 1.44 (1.24-1.67) | 96 |

LT50 is the estimated time to kill 50% of the insects based on Probit analysis, % mortality is Abbott's corrected and DAT is the days after treatment.

The combination of Yersinia entomophaga strain O33ZDX with Bacillus thuringiensis subspecies kurstaki resulted in a significantly lower LT50 than either active alone. The data in the above table also show the percent mortality of tobacco budworm at 2 days after treatment, for Ye O33ZDX alone, for Btk alone, and for combinations of Ye O33ZDX and Btk.

### Example 6

### Yersinia Entomophaga Strain O43NEW with Bacillus Thuringiensis Subspecies Kurstaki Against European Corn Borer

Cabbage leaf disks were dipped in either Yersinia entomophaga (Ye) strain O43NEW at a concentration of 1×10⁷ CFU/mL in phosphate buffer, Bacillus thuringiensis subspecies kurstaki (Btk) at a concentration of 1×10⁷ CFU/mL in phosphate buffer, or a 1:1 combination of these two actives. Controls were dipped in phosphate buffer. After the cabbage disks had dried for 1 hr, a single 2^{nd} instar European corn borer was added to each individual cabbage disk. A total of 24 insects were evaluated for each treatment.

TABLE 16
Mortality of 2^{nd} instar European corn borer with B. thuringiensis
subspecies kurstaki (Btk), Y. entomophaga (Ye)
strain O43NEW, and a combination of both

| | LT50 (95% confidence | % Mortality |
|---|---|---|
| Treatment | intervals) in days | (2DAT) |
| Btk (ABTS-351) at 1 × 10⁷ | 2.04 (1.82-2.28) | 42 |
| CFU/mL | | |
| Ye O43NEW at 1 × 10⁷ CFU/mL | 3.09 (2.82-3.38) | 14 |
| Btk + Ye O43NEW | 1.45 (1.27-1.65) | 88 |

LT50 is the estimated time to kill 50% of the insects based on Probit analysis, % mortality is Abbott's corrected and DAT is the days after treatment.

The combination of Yersinia entomophaga strain O43NEW with Bacillus thuringiensis subspecies kurstaki resulted in a significantly lower LT50 than either active alone. The data in the above table also show the percent mortality of European corn borer at 2 days after treatment, for Ye O43NEW alone, for Btk alone, and for combinations of Ye O43NEW and Btk.

### Example 7

### Yersinia Entomophaga Strain O43NEW with Bacillus Thuringiensis Subspecies Tenebrionis Against Colorado Potato Beetle

Tomato leaf disks were dipped in either Yersinia entomophaga (Ye) strain O43NEW at a concentration of 1×10⁶ CFU/mL in phosphate buffer, Bacillus thuringiensis subspecies tenebrionis IBL1410 (Btt) at a concentration of 1×10⁶ CFU/mL in phosphate buffer, or a 1:1 combination of these two actives. Controls were dipped in phosphate buffer. After the tomato disks had dried for 1 hr, a single 3rd instar Colorado potato beetle larva was added to each individual tomato disk. A total of 20 insects were evaluated for each treatment.

TABLE 17
Mortality of 3^{rd} instar Colorado potato beetle treated with
B. thuringiensis subspecies tenebrionis IBL1410 (Btt),
Y. entomophaga (Ye) strain O43NEW, and a combination of both

| | LT50 (95% confidence | % Mortality | % Mortality | % Mortality |
|---|---|---|---|---|
| Treatment | intervals) in days | (5DAT) | (6DAT) | (7DAT) |
| Btt at 1 × 10⁶ CFU/mL | 10.39 (9.54-11.33) | 0 | 0 | 19 |
| Ye O43NEW at 1 × 10⁶ | 7.42 (6.81-8.09) | 0 | 1 | −11 |
| CFU/mL | | | | |
| Btt + Ye O43NEW | 6.90 (6.33-7.52) | 5 | 22 | 50 |

LT50 is the estimated time to kill 50% of the insects based on Probit analysis, % mortality is Abbott's corrected and DAT is the days after treatment.
Negative mortality data (e.g., −11) indicates that mortality in this treatment was lower than control mortality based on Abbott's formula, which subtracts control mortality from treated mortality.

The data in the above table show the percent mortality of Colorado potato beetle at 5, 6 and 7 days after treatment, for Ye O43NEW alone, for Btt alone, and for combinations of Ye and Btt.

Another experiment was performed similarly, except that the amounts of Yersinia entomophaga strain O43NEW and Bacillus thuringiensis subspecies tenebrionis were different than those used in the previous experiment (see table below). A total of 20 insects were evaluated for each treatment. The data from that experiment are shown in the table below.

TABLE 18
Mortality of 3rd instar Colorado potato beetle treated with
B. thuringiensis subspecies tenebrionis IBL1410 (Btt),
Y. entomophaga (Ye) strain O43NEW, and a combination of both

| | LT50 (95% confidence | % Mortality | % Mortality | % Mortality |
|---|---|---|---|---|
| Treatment | intervals) in days | (5DAT) | (7DAT) | (9DAT) |
| Btt at 1 × 10⁷ CFU/mL | 69.3 (39.8-133.1) | −6 | −6 | −6 |
| Ye O43NEW at 1 × 10⁵ | 38.1 (23.7-66.1) | 5 | 5 | 5 |
| CFU/mL | | | | |
| Btt + Ye O43NEW | 12.0 (8.2-18.1) | 18 | 27 | 43 |

LT50 is the estimated time to kill 50% of the insects based on Probit analysis, % mortality is Abbott's corrected and DAT is the days after treatment.
Negative mortality data (e.g., −6) indicates that mortality in this treatment was lower than control mortality based on Abbott's formula, which subtracts control mortality from treated mortality.

The combination of Yersinia entomophaga strain O43NEW with Bacillus thuringiensis subspecies tenebrionis resulted in a significantly lower LT50 than either active alone. The data in the above table also show the percent mortality of Colorado potato beetle at 5, 7 and 9 days after treatment, for Ye O43NEW alone, for Btt alone, and for combinations of Ye and Btt.

Another experiment was performed similarly, except that the amount of Yersinia entomophaga strain O43NEW was different than that used in the previous experiment (see table below). A total of 20 insects were evaluated for each treatment. The data from that experiment are shown in the table below.

TABLE 19
Mortality of 3rd instar Colorado potato beetle treated with B. thuringiensis subspecies
tenebrionis IBL1410 (Btt), Y. entomophaga (Ye) strain O43NEW, and a combination of both

| | LT50 (95% confidence | % Mortality | % Mortality | % Mortality | % Mortality |
|---|---|---|---|---|---|
| Treatment | intervals) in days | (5DAT) | (6DAT) | (7DAT) | (9DAT) |
| Btt at 1 × 10⁷ | 69.3 (39.8-133.1) | −6 | −6 | −6 | −6 |
| CFU/mL | | | | | |
| Ye O43NEW at | 61.8 (36.1-116.0) | −6 | −6 | −6 | −6 |
| 1 × 10⁶ CFU/mL | | | | | |
| Btt + Ye | 12.0 (8.2-18.1) | 27 | 36 | 49 | 68 |
| O43NEW | | | | | |

LT50 is the estimated time to kill 50% of the insects based on Probit analysis, % mortality is Abbott's corrected and DAT is the days after treatment.
Negative mortality data (e.g., −6) indicates that mortality in this treatment was lower than control mortality based on Abbott's formula, which subtracts control mortality from treated mortality.

The combination of Yersinia entomophaga strain O43NEW with Bacillus thuringiensis subspecies tenebrionis resulted in a significantly lower LT50 than either active alone. The data in the above table also show the percent mortality of Colorado potato beetle at 5, 6, 7 and 9 days after treatment, for Ye O43NEW alone, for Btt alone, and for combinations of Ye and Btt.

Another experiment was performed similarly, except that the amount of Yersinia entomophaga strain O43NEW was different than that used in the previous experiment (see table below). A total of 20 insects were evaluated for each treatment. The data from that experiment are shown in the table below.

TABLE 20
Mortality of 3rd instar Colorado potato beetle treated with B. thuringiensis subspecies
tenebrionis IBL1410 (Btt), Y. entomophaga (Ye) strain O43NEW, and a combination of both

| | LT50 (95% confidence | % Mortality | % Mortality | % Mortality | % Mortality |
|---|---|---|---|---|---|
| Treatment | intervals) in days | (5DAT) | (6DAT) | (7DAT) | (9DAT) |
| Btt at 1 × 10⁷ | 69.3 (39.8-133.1) | −6 | −6 | −6 | −6 |
| CFU/mL | | | | | |
| Ye O43NEW at | 20.8 (13.6-33.4) | 5 | 15 | 26 | 26 |
| 1 × 10⁷ CFU/mL | | | | | |
| Btt + Ye O43NEW | 2.0 (1.3-3.1) | 62 | 68 | 81 | 94 |

LT50 is the estimated time to kill 50% of the insects based on Probit analysis, % mortality is Abbott's corrected and DAT is the days after treatment.
Negative mortality data (e.g., −6) indicates that mortality in this treatment was lower than control mortality basedon Abbott's formula, which subtracts control mortality from treated mortality.

The combination of Yersinia entomophaga strain O43NEW with Bacillus thuringiensis subspecies tenebrionis resulted in a significantly lower LT50 than either active alone. The data in the above table also show the percent mortality of Colorado potato beetle at 5, 6, 7 and 9 days after treatment, for Ye O43NEW alone, for Btt alone, and for combinations of Ye and Btt.

### Example 8

### Yersinia Entomophaga with Bacillus Thuringiensis Subspecies Israelensis Against Diptera

Diet or food that is consumable by a fly of the order Diptera, is dipped in Yersinia entomophaga, in Bacillus thuringiensis subspecies israelensis, or in both Yersinia entomophaga and Bacillus thuringiensis subspecies israelensis. Controls are dipped in phosphate buffer. After the diet or fool had dried, fly larvae or eggs are added to each individual diet or food source.

The data will show that the LT50 for insect killing of the combination of Yersinia and Bacillus is significantly lower than the LT50 of either bacterium alone. Data on percent mortality of the insects with the bacterial combination will show unexpected results as compared to mortality due to either bacterium alone.

### Example 9

### Summary of Insect Killing (Using % Mortality) Using Combinations of Yersinia Entomophaga and Bacillus Thuringiensis

The table below is a summary of selected, but representative data, from Examples 2-7. Each row of the table includes data from a selected experiment described in one of the Examples 2-7. The columns in the table, from left to right, indicate the particular insect that the bacteria were tested against, the specific Yersinia entomophaga isolate and amount used, the subspecies of Bacillus thuringiensis and amount used, the % mortality of the insect with Yersinia entomophaga alone, the % mortality of the insect with Bacillus thuringiensis alone, the % mortality with the Yersina+Bacillus combination, a calculated index of performance for the combination as compared to the individual components of the combination tested alone (performance inde=mortality of insects exposed to the combination of bacteria divided by the cumulative mortality of insects exposed to the bacteria individually), and the time point of insect killing (days after treatment) at which the data to calculate the performance index were collected.

TABLE 21
Summary of % mortality data from Examples 2-7 using combinations of Yersinia and Bacillus

| | | | | | % | | |
|---|---|---|---|---|---|---|---|
| | Y. entomophaga | B. thuringiensis | % | % | Mortality | | |
| | Isolate | Subspecies and | Mortality | Mortality | Yersinia + | Performance | |
| Insect | (CFU/ml) | Amount | Yersinia | Bacillus | Bacillus | Index | DAT |
| Black cutworm | O43NEW | kurstaki, | 6 | 39 | 65 | 1.4 | 2 |
| (Table 1) | (1 × 10⁵) | DiPel ® at | | | | | |
| | | 0.12% w/w | | | | | |
| Cabbage looper | O43NEW | kurstaki, at | 17 | 25 | 96 | 2.3 | 2 |
| (Table 3) | (1 × 10⁷) | 1 × 10⁷ CFU/ml | | | | | |
| Cabbage looper | O23ZMJ | kurstaki, at | 17 | 33 | 100 | 2.0 | 2 |
| (Table 4) | (1 × 10⁷) | 1 × 10⁷ CFU/ml | | | | | |
| Cabbage looper | O24G3R | kurstaki, at | 15 | 33 | 88 | 1.8 | 2 |
| (Table 5) | (1 × 10⁷) | 1 × 10⁷ CFU/ml | | | | | |
| Cabbage looper | O348UX | kurstaki, at | 8 | 33 | 96 | 2.3 | 2 |
| (Table 6) | (1 × 10⁷) | 1 × 10⁷ CFU/ml | | | | | |
| Cabbage looper | O33ZDX | kurstaki, at | 8 | 33 | 80 | 2.0 | 2 |
| (Table 7) | (1 × 10⁷) | 1 × 10⁷ CFU/ml | | | | | |
| Fall armyworm | O43NEW | kurstaki, | 13 | 25 | 60 | 1.6 | 5 |
| (Table 8) | (1 × 10⁷) | DiPel ® at | | | | | |
| | | 0.12% w/w | | | | | |
| Tobacco | O43NEW | kurstaki, | 30 | 20 | 80 | 1.6 | 5 |
| budworm | (1 × 10⁴) | DiPel ® at | | | | | |
| (Table 9) | | 0.0012% w/w | | | | | |
| Tobacco | O43NEW | kurstaki, at | 25 | 21 | 96 | 2.1 | 2 |
| budworm | (1 × 10⁷) | 1 × 10⁷ CFU/ml | | | | | |
| (Table 11) | | | | | | | |
| Tobacco | O43ZMJ | kurstaki, at | 4 | 9 | 96 | 7.4 | 2 |
| budworm | (1 × 10⁷) | 1 × 10⁷ CFU/ml | | | | | |
| (Table 12) | | | | | | | |
| Tobacco | O24G3R | kurstaki, at | 21 | 9 | 83 | 2.8 | 2 |
| budworm | (1 × 10⁷) | 1 × 10⁷ CFU/ml | | | | | |
| (Table 13) | | | | | | | |
| Tobacco | O348UX | kurstaki, at | 8 | 9 | 100 | 5.9 | 2 |
| budworm | (1 × 10⁷) | 1 × 10⁷ CFU/ml | | | | | |
| (Table 14) | | | | | | | |
| Tobacco | O348UX | kurstaki, at | 25 | 9 | 96 | 2.8 | 2 |
| budworm | (1 × 10⁷) | 1 × 10⁷ CFU/ml | | | | | |
| (Table 15) | | | | | | | |
| European corn | O43NEW | kurstaki, at | 14 | 42 | 88 | 1.6 | 2 |
| borer (Table 16) | (1 × 10⁷) | 1 × 10⁷ CFU/ml | | | | | |
| Colorado potato | O43NEW | tenebrionis, at | 1 | 0 | 11 | 22.0¹ | 6 |
| beetle (Table 17) | (1 × 10⁶) | 1 × 10⁶ CFU/ml | | | | | |
| Colorado potato | O43NEW | tenebrionis, at | 5 | −6 | 27 | 4.5¹ | 7 |
| beetle (Table 18) | (1 × 10⁵) | 1 × 10⁷ CFU/ml | | | | | |
| Colorado potato | O43NEW | tenebrionis, at | −6 | −6 | 36 | 13.0¹ | 6 |
| beetle (Table 19) | (1 × 10⁶) | 1 × 10⁷ CFU/ml | | | | | |
| Colorado potato | O43NEW | tenebrionis, at | 15 | −6 | 68 | 4.3¹ | 6 |
| beetle (Table 20) | (1 × 10⁷) | 1 × 10⁷ CFU/ml | | | | | |

¹Negative numbers for mortality indicate that mortality was lower than control mortality based on Abbott's formula. A value of 0 for mortality indicates that mortality was equal to control mortality. In these cases, a value of 1 was used for calculating Performance Index.

### Example 10

### Summary of Insect Killing (Using LT50) Using Combinations of Yersinia Entomophaga and Bacillus Thuringiensis

The table below is a summary of the data from Examples 2-7. Each row of the table includes data from a selected experiment described in one of the Examples 2-7. The columns in the table, from left to right, indicate the particular insect that the bacteria were tested against, the specific Yersinia entomophaga isolate and amount used, the subspecies of Bacillus thuringiensis and amount used, the LT50 of the insect exposed to the Yersinia alone, the LT50 of the insect exposed to the Bacillus alone, the LT50 of the insect exposed to the combination of Yersinia+Bacillus, the reduction in LT50 (equal to the lowest LT50 of a single active minus the LT50 of the combination), and the % reduction in the LT50 (equal to the LT50 reduction divided by the lowest LT50 of a single active×100%).

TABLE 22
Summary of LT50 data from Examples 2-7 using combinations of Yersinia and Bacillus

| | Yersinia | Bacillus | | | | | |
|---|---|---|---|---|---|---|---|
| | entomophaga | thuringiensis | | | LT50 | LT50 | LT50 |
| | Isolate | Subspecies and | LT50 | LT50 | Yersinia + | reduction | reduction |
| Insect | (CFU/ml) | Amount | Yersinia | Bacillus | Bacillus | (days) | (%) |
| Black | O43NEW | kurstaki, DiPel ® | 6.95 | 3.84 | 2.10 | 1.74 | 45 |
| cutworm | (1 × 10⁵) | at 0.12% w/w | | | | | |
| (Table 1) | | | | | | | |
| Cabbage | O43NEW | kurstaki, DiPel ® | 8.43 | 8.71 | 5.54 | 2.89 | 34 |
| looper | (1 × 10³) | at 0.0012% w/w | | | | | |
| (Table 2) | | | | | | | |
| Cabbage | O43NEW | kurstaki, at 1 × 10⁷ | 2.48 | 3.01 | 1.12 | 1.36 | 55 |
| looper | (1 × 10⁷) | CFU/ml | | | | | |
| (Table 3) | | | | | | | |
| Cabbage | O23ZMJ | kurstaki, at 1 × 10⁷ | 2.61 | 2.66 | 1.34 | 1.27 | 49 |
| looper | (1 × 10⁷) | CFU/ml | | | | | |
| (Table 4) | | | | | | | |
| Cabbage | O24G3R | kurstaki, at 1 × 10⁷ | 2.43 | 2.66 | 1.41 | 1.02 | 42 |
| looper | (1 × 10⁷) | CFU/ml | | | | | |
| (Table 5) | | | | | | | |
| Cabbage | O348UX | kurstaki, at 1 × 10⁷ | 2.56 | 2.66 | 1.43 | 1.13 | 44 |
| looper | (1 × 10⁷) | CFU/ml | | | | | |
| (Table 6) | | | | | | | |
| Cabbage | O33ZDX | kurstaki, at 1 × 10⁷ | 2.40 | 2.66 | 1.77 | 0.63 | 26 |
| looper | (1 × 10⁷) | CFU/ml | | | | | |
| (Table 7) | | | | | | | |
| Fall | O43NEW | kurstaki, DiPel ® | 7.07 | 6.77 | 4.35 | 2.42 | 36 |
| armyworm | (1 × 10⁷) | at 0.12% w/w | | | | | |
| (Table 8) | | | | | | | |
| Tobacco | O43NEW | kurstaki, DiPel ® | 7.83 | 8.91 | 4.64 | 3.19 | 41 |
| budworm | (1 × 10⁴) | at 0.0012% w/w | | | | | |
| (Table 9) | | | | | | | |
| Tobacco | O43NEW | kurstaki, DiPel ® | 4.59 | 5.99 | 3.04 | 1.55 | 34 |
| budworm | (1 × 10⁵) | at 0.0012% w/w | | | | | |
| (Table 10) | | | | | | | |
| Tobacco | O43NEW | kurstaki, at 1 × 10⁷ | 2.24 | 2.23 | 1.32 | 0.91 | 41 |
| budworm | (1 × 10⁷) | CFU/ml | | | | | |
| (Table 11) | | | | | | | |
| Tobacco | O43ZMJ | kurstaki, at 1 × 10⁷ | 2.64 | 2.60 | 1.40 | 1.20 | 46 |
| budworm | (1 × 10⁷) | CFU/ml | | | | | |
| (Table 12) | | | | | | | |
| Tobacco | O24G3R | kurstaki, at 1 × 10⁷ | 3.24 | 2.60 | 1.36 | 1.24 | 48 |
| budworm | (1 × 10⁷) | CFU/ml | | | | | |
| (Table 13) | | | | | | | |
| Tobacco | O348UX | kurstaki, at 1 × 10⁷ | 3.02 | 2.60 | 1.24 | 1.36 | 52 |
| budworm | (1 × 10⁷) | CFU/ml | | | | | |
| (Table 14) | | | | | | | |
| Tobacco | O348UX | kurstaki, at 1 × 10⁷ | 2.70 | 2.60 | 1.44 | 1.16 | 45 |
| budworm | (1 × 10⁷) | CFU/ml | | | | | |
| (Table 15) | | | | | | | |
| European | O43NEW | kurstaki, at 1 × 10⁷ | 3.09 | 2.04 | 1.45 | 0.59 | 29 |
| corn borer | (1 × 10⁷) | CFU/ml | | | | | |
| (Table 16) | | | | | | | |
| Colorado | O43NEW | tenebrionis, at | 7.42 | 10.39 | 6.90 | 0.52 | 7 |
| potato beetle | (1 × 10⁶) | 1 × 10⁶ CFU/ml | | | | | |
| (Table 17) | | | | | | | |
| Colorado | O43NEW | tenebrionis, at | 38.1 | 69.3 | 12.0 | 26.1 | 69 |
| potato beetle | (1 × 10⁵) | 1 × 10⁷ CFU/ml | | | | | |
| (Table 18) | | | | | | | |
| Colorado | O43NEW | tenebrionis, at | 61.8 | 69.3 | 12.0 | 49.8 | 81 |
| potato beetle | (1 × 10⁶) | 1 × 10⁷ CFU/ml | | | | | |
| (Table 19) | | | | | | | |
| Colorado | O43NEW | tenebrionis, at | 20.8 | 69.3 | 2.0 | 18.8 | 90 |
| potato beetle | (1 × 10⁷) | 1 × 10⁷ CFU/ml | | | | | |
| (Table 20) | | | | | | | |

### Example 11

### Summary of Insect Killing (Using LT50) Using Combinations of Yersinia Entomophaga and Bacillus Thuringiensis

Cabbage loopers instar) were provided no food and no water for 16 hours and held at 95% relative humidity. Then they were provided with a 5 uL droplet of phosphate buffer containing blue food coloring to help observe consumption. This droplet contained either Yersinia entomophaga (Ye) strain O43NEW, Bacillus thuringiensis subspecies kurstaki (Btk) as DiPel®, or a combination of these two actives according to Table 23. Controls were only given phosphate buffer containing the blue food coloring. The cabbage loopers were given 1 hr to consume the droplet and then transferred to a cabbage leaf disk and held at 25 C and 87% relative humidity. A total of 10 insects were evaluated for each treatment.

TABLE 23
Mortality of 3^{rd} instar cabbage looper treated with B. thuringiensis subspecies
kurstaki (Btk), Y. entomophaga (Ye) strain O43NEW, and a combination of both

| | LT50 (95% | | | | | | | |
|---|---|---|---|---|---|---|---|---|
| | confidence | % | % | % | % | % | % | % |
| | intervals) | Mortality | Mortality | Mortality | Mortality | Mortality | Mortality | Mortality |
| Treatment | in days | (1DAT) | (2DAT) | (6DAT) | (7DAT) | (8DAT) | (9DAT) | (12DAT) |
| Ye at 1 × 10⁷ | 17.5 (12.0-27.3) | 0 | 0 | 10 | 10 | 10 | 13 | 29 |
| CFU/mL | | | | | | | | |
| Btk | 0.48 (0.23-0.91) | 70 | 100 | 100 | 100 | 100 | 100 | 100 |
| (DiPel ®) at | | | | | | | | |
| 0.012% w/w | | | | | | | | |
| Btk | 0.27 (0.09-0.67) | 90 | 100 | 100 | 100 | 100 | 100 | 100 |
| (DiPel ®) at | | | | | | | | |
| 0.012% w/w + | | | | | | | | |
| Ye at 1 × 10⁷ | | | | | | | | |
| CFU/mL | | | | | | | | |
| Btk | 32.9 (19.5-62.5) | 0 | 0 | 0 | 0 | 0 | −25 | 14 |
| (DiPel ®) at | | | | | | | | |
| 0.0012% | | | | | | | | |
| w/w | | | | | | | | |
| Btk | 3.2 (2.2-4.3) | 40 | 50 | 50 | 60 | 80 | 88 | 86 |
| (DiPel ®) at | | | | | | | | |
| 0.0012% | | | | | | | | |
| w/w + | | | | | | | | |
| Ye at 1 × 10⁷ | | | | | | | | |
| CFU/mL | | | | | | | | |
| Btk | 25.8 (16.4-44.9) | 0 | 0 | 0 | 0 | 10 | 0 | 0 |
| (DiPel ®) at | | | | | | | | |
| 0.00012% | | | | | | | | |
| w/w | | | | | | | | |
| Btk | 7.1 (5.2-9.8) | 10 | 20 | 20 | 20 | 50 | 50 | 100 |
| (DiPel ®) at | | | | | | | | |
| 0.00012% | | | | | | | | |
| w/w + | | | | | | | | |
| Ye at 1 × 10⁷ | | | | | | | | |
| CFU/mL | | | | | | | | |
| Btk | 18.9 (12.8-30.2) | 0 | 0 | 0 | 0 | 10 | 13 | 43 |
| (DiPel ®) at | | | | | | | | |
| 0.000012% | | | | | | | | |
| w/w | | | | | | | | |
| Btk | 8.1 (5.9-11.2) | 0 | 10 | 20 | 20 | 50 | 63 | 86 |
| (DiPel ®) at | | | | | | | | |
| 0.000012% | | | | | | | | |
| w/w + | | | | | | | | |
| Ye at 1 × 10⁷ | | | | | | | | |
| CFU/mL | | | | | | | | |

LT50 is the estimated time to kill 50% of the insects based on Probit analysis, % mortality is Abbott's corrected and DAT is the days after treatment
Negative numbers for mortality indicate that mortality was lower than control mortality based on Abbott's formula. In these cases, a value of 1 was used for calculating Performance Index.

The combination of Yersinia entomophaga strain O43NEW with Bacillus thuringiensis subspecies kurstaki resulted in a significantly lower LT50 than either active alone. The data in the above table also show the percent mortality of Colorado potato beetle at 1, 2, 6, 7, 8, 9, and 12 days after treatment, for Ye O43NEW alone, for Btk alone, and for combinations of Ye and Btk.

1-63. (canceled)

64. A method comprising:
applying a Bacillus thuringiensis pesticide to a plant or plant part in an amount effective to produce a first anticipated insecticidal effect; and
applying a Yersinia entomophaga pesticide to said plant or plant part in an amount effective to produce a second anticipated insecticidal effect,
wherein said Bacillus thuringiensis pesticide comprises one or more Bacillus thuringiensis strains, one or more cell-free Bacillus thuringiensis culture filtrates and/or one or more isolated Bacillus thuringiensis toxins,
wherein said Yersinia entomophaga pesticide comprises one or more Yersinia entomophaga strains, one or more Yersinia entomophaga cell-free culture filtrates and/or one or more isolated Yersinia entomophaga toxins, and
wherein application of said Bacillus thuringiensis pesticide and said Yersinia entomophaga pesticide to said plant or plant part produces an unexpected cumulative insecticidal effect relative to said first anticipated insecticidal effect and said second anticipated insecticidal effect.

65. The method of claim 64, wherein said Yersinia entomophaga pesticide comprises at least one Yersinia entomophaga strain having a whole genome sequence that is at least 99.5% identical to the whole genome sequence of Yersinia entomophaga MH96.

66. The method of claim 64, wherein said Yersinia entomophaga pesticide comprises Yersinia entomophaga MH96, Yersinia entomophaga NRRL B-67598, Yersinia entomophaga NRRL B-67599, Yersinia entomophaga NRRL B-67600 and/or Yersinia entomophaga NRRL B-67601.

67. The method of claim 64, wherein said Yersinia entomophaga pesticide comprises a cell-free filtrate of a culture comprising at least one Yersinia entomophaga strain having a whole genome sequence that is at least 99.5% identical to the whole genome sequence of Yersinia entomophaga MH96.

68. The method of claim 64, wherein said Yersinia entomophaga pesticide comprises a cell-free filtrate of a culture comprising Yersinia entomophaga MH96, Yersinia entomophaga NRRL B-67598, Yersinia entomophaga NRRL B-67599, Yersinia entomophaga NRRL B-67600 and/or Yersinia entomophaga NRRL B-67601.

69. The method of claim 64, wherein said Yersinia entomophaga pesticide comprises one or more toxins derived from a Yersinia entomophaga strain having a whole genome sequence that is at least 99.5% identical to the whole genome sequence of Yersinia entomophaga MH96.

70. The method of claim 64, wherein said Yersinia entomophaga pesticide comprises one or more toxins derived from Yersinia entomophaga MH96, Yersinia entomophaga NRRL B-67598, Yersinia entomophaga NRRL B-67599, Yersinia entomophaga NRRL B-67600 and/or Yersinia entomophaga NRRL B-67601.

71. The method of claim 64, wherein said unexpected cumulative insecticidal effect comprises an unexpected magnitude of insecticidal activity against an insect.

72. The method of claim 64, wherein said first anticipated insecticidal effect comprises a first anticipated percent mortality of an insect one day after treatment,
wherein said second anticipated insecticidal effect comprises a second anticipated percent mortality of said insect one day after treatment, and
wherein said unexpected cumulative insecticidal effect comprises a percent mortality of said insect one day after treatment that exceeds the numerical sum of said first anticipated percent mortality of said insect one day after treatment and said second anticipated percent mortality of said insect one day after treatment.

73. The method of claim 64, wherein said first anticipated insecticidal effect comprises a first anticipated percent mortality of an insect two days after treatment,
wherein said second anticipated insecticidal effect comprises a second anticipated percent mortality of said insect two days after treatment, and
wherein said unexpected cumulative insecticidal effect comprises a percent mortality of said insect two days after treatment that exceeds the numerical sum of said first anticipated percent mortality of said insect two days after treatment and said second anticipated percent mortality of said insect two days after treatment.

74. The method of claim 64, wherein said first anticipated insecticidal effect comprises a first anticipated percent mortality of an insect three days after treatment,
wherein said second anticipated insecticidal effect comprises a second anticipated percent mortality of said insect three days after treatment, and
wherein said unexpected cumulative insecticidal effect comprises a percent mortality of said insect three days after treatment that exceeds the numerical sum of said first anticipated percent mortality of said insect three days after treatment and said second anticipated percent mortality of said insect three days after treatment.

75. The method of claim 64, wherein said first anticipated insecticidal effect comprises a first anticipated percent mortality of an insect five days after treatment,
wherein said second anticipated insecticidal effect comprises a second anticipated percent mortality of said insect five days after treatment, and
wherein said unexpected cumulative insecticidal effect comprises a percent mortality of said insect five days after treatment that exceeds the numerical sum of said first anticipated percent mortality of said insect five days after treatment and said second anticipated percent mortality of said insect five days after treatment.

76. The method of claim 64, wherein said first anticipated insecticidal effect comprises a first anticipated percent mortality of an insect seven days after treatment,
wherein said second anticipated insecticidal effect comprises a second anticipated percent mortality of said insect seven days after treatment, and
wherein said unexpected cumulative insecticidal effect comprises a percent mortality of said insect seven days after treatment that exceeds the numerical sum of said first anticipated percent mortality of said insect seven days after treatment and said second anticipated percent mortality of said insect seven days after treatment.

77. The method of claim 64, wherein said first anticipated insecticidal effect comprises a first anticipated percent mortality of an insect nine days after treatment,
wherein said second anticipated insecticidal effect comprises a second anticipated percent mortality of said insect nine days after treatment, and
wherein said unexpected cumulative insecticidal effect comprises a percent mortality of said insect nine days after treatment that exceeds the numerical sum of said first anticipated percent mortality of said insect nine days after treatment and said second anticipated percent mortality of said insect nine days after treatment.

79. The method of claim 71, wherein said insect is selected from the group consisting of Coleoptera, Diptera, Hymenoptera, Lepidoptera, Orthoptera and Thysanoptera.

80. The method of claim 64, wherein said Bacillus thuringiensis pesticide and said Yersinia entomophaga pesticide are applied to said plant or plant part concurrently.

81. The method of claim 64, wherein said Bacillus thuringiensis pesticide and said Yersinia entomophaga pesticide are sprayed onto said plant or plant part.

82. The method of claim 64, wherein said plant or plant part comprises plant foliage.

83. A plant or plant part treated according to the method of claim 64, said Bacillus thuringiensis pesticide and said Yersinia entomophaga pesticide being present on one or more surfaces of said plant or plant part.
